(19) United States
(12) Patent Application Publication
(10) Pub. No.: US 2021/0046182 A1
(43) Pub. Date: Feb. 18, 2021

(54) HIGH CONCENTRATION ANTI-C5 FORMULATIONS

(71) Applicant: Regeneron Pharmaceuticals, Inc., Tarrytown, NY (US)

(72) Inventors: Mary Kleppe, Milford, CT (US); Mayank Patel, New City, NY (US); Xiaolin Tang, Old Tappan, NJ (US)

(21) Appl. No.: 16/993,434

(22) Filed: Aug. 14, 2020

Related U.S. Application Data
(60) Provisional application No. 62/888,086, filed on Aug. 16, 2019.

(51) Int. Cl. A61K 39/395; C07K 16/18; A61K 47/18; A61K 47/26; A61K 45/06; A61K 9/00

(52) U.S. Cl. CPC A61K 39/3955; C07K 16/18; A61K 9/0019; A61K 47/26; A61K 45/06; A61K 47/183

(57) ABSTRACT

The present disclosure includes high concentration, low viscosity pharmaceutical formulations that include an anti-C5 antibody or antigen-binding fragment thereof and arginine. Such formulations may be provided in association with an RNAi molecule such as cemdisiran. Methods of treating C5-associated diseases such as PNH and aHUS are also provided.

The present application claims the benefit of U.S. Provisional Patent Application No. 62/888,086, filed Aug. 16, 2019, which is hereby incorporated by reference in its entirety for all purposes.

The sequence listing of the present application is submitted electronically as an ASCII formatted sequence listing with a file name “10643seqlist”, creation date of Aug. 11, 2020, and a size of 136 Kb. This sequence listing submitted is part of the specification and is hereby incorporated by reference in its entirety.

### FIELD

The field of the disclosure relates to pharmaceutical formulations comprising antibodies and antigen-binding fragments thereof and methods of treatment using such formulations.

### BACKGROUND

Pharmaceutical formulations for delivering a high dose of antibody or other polypeptide in a moderate volume present a challenge due to the high viscosities that result. As the concentration of antibody increases, the viscosity of the formulation typically increases exponentially. Yadav et al., J Pharm Sci. 99 (12) 4812-29 (2010). For example, Cimzia contains the PEGylated Fab′ fragment at a concentration of 200 mg/ml and a viscosity of about 80 cP (a relatively high viscosity). See “Innovative Drug Delivery Technology to Meet Evolving Need of Biologics & Small Molecules,” ONdrugDelivery Magazine, Issue 56 (March 2015), pp 4-6.

Viscous solutions require high injection force, through a needle, to administer the drug and may also require a prolonged injection time. Pain and discomfort experienced by the patient during long injection times can have a negative impact on compliance and adherence to the medication. Moreover, the potential for product loss that could result from highly viscous solutions sticking to the contact surface of the primary packaging can also be a problem. If drug delivery is through an autoinjector, the challenge will be to ensure that the device can produce the required force to function properly throughout its shelf life, hence necessitating extensive modeling and accelerated aging to simulate the high stress placed on the device.

Acceptable subcutaneous (SQ or SC) anti-C5 therapeutic antibody formulations are particularly difficult to develop. Because the concentration of C5 in plasma is relatively high (approximately 80 μg/mL), large amounts of antibody are typically needed to block at a therapeutic level. Holers, Annu Rev Immunol 32: 433-459 (2014). Subcutaneous administration is typically preferred due to patient convenience. SQ injections can usually be done by the patient himself whereas intravenous (IV) administration must be done by a doctor/in the clinic. For example, eculizumab has been approved for treatment of various C5-mediated diseases. Patients are dosed with a large amount (900-1200 mg) of eculizumab every other week, and this huge dosage requires IV administration. Holers (2014). Another approved therapeutic anti-C5 antibody, ravulizumab (sold as Ultomoris), is dosed, IV at even higher levels, at 2400-3000 mg. SQ Ultomoris is dosed weekly at 700 mg from a 100 mg/ml formulation (7 ml dosage volume given in two separate injections). Alexion Pharmaceuticals, Inc., Investor Day presentation (Mar. 20, 2019). As discussed, high SQ dosage volumes present problems, for example, due to the extended period of time required for the full dose to be injected. With a device capable of a 1 ml/minute injection rate, 7 minutes would be required. During this time, errors can occur during injection, for example, interruption of the injection.

### SUMMARY

The present invention provides a pharmaceutical formulation comprising about 150 or 200 mg/ml or more antigen-binding protein (e.g., antibody or antigen-binding fragment thereof) that binds specifically to C5 (H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4HI2169P; H4H12170P; H4H12171P; H4H12175P; H4H12176P2; H4H12177P2; H4H12183P2; H2M11682N; H2M11684N; H2M11694N; H2M11695N; ravulizumab; crovalimab, eculizumab; tesidolumab or mubodina); and a pharmaceutically acceptable carrier comprising: buffer (e.g., phosphate buffer, acetate buffer, citrate buffer, histidine buffer or imidazole buffer); arginine (for example, L-arginine HCl, e.g., 50-100 mM, e.g., 100 mM); water; and, optionally, an oligosaccharide (for example, sucrose, mannitol, dextrose, glycerol, TMAO (trimethylamine N-oxide), trehalose, ethylene glycol, glycine betaine, xylitol or sorbitol, e.g., 2%); and optionally, a non-ionic detergent (e.g., a polyoxyethylene-based detergent or a glycosidic compound-based detergent, polysorbate-20, polysorbate-80 or tween-20), pH of up to about 6.1, e.g., 5-6, e.g., 5.8; and a viscosity of about 14, 14.3 or 15 cP (20° C.) or less. In an embodiment of the invention, the anti-C5 antigen-binding protein is pozelimab. In an embodiment of the invention, the formulation comprises about 200 mg/ml antibody that binds specifically to human C5 (e.g., pozelimab); about 20 mM histidine buffer; about 100 mM L-arginine; about 2% sucrose; about 0.15% polysorbate-80 and water, pH 5.8±0.2. In an embodiment of the invention, the pharmaceutical formulation is aqueous (e.g., suitable for intravenous and/or subcutaneous administration) and comprises H4H12166P (e.g., about 200 mg/mL), histidine (e.g., histidine-HCl; e.g., about 20 mM), pH about 5.8, arginine (e.g., about 100 mM; e.g., L-arginine or L-arginine hydrochloride), a polyol such as sucrose (e.g., about 2% (w/v)), and a non-ionic surfactant such as polysorbate (e.g., polysorbate 80; e.g., about 0.15% (w/v)). In an embodiment of the invention, the pharmaceutical formulation is aqueous (e.g., suitable for intravenous and/or subcutaneous administration) and comprises H4H12166P (e.g., about 200 mg/mL, 200 mg/ml±20 mg/ml or 180-210 mg/ml), histidine (e.g., histidine-HCl; e.g., about 10-20 or 10-24 mM), pH about 5.5±0.6, and arginine (e.g., about 100 mM±20 mM; e.g., L-arginine, L-arginine HCl or L-arginine monohydrochloride), optionally, a polyol such as sucrose (e.g., about 2% (w/v)), and optionally, a non-ionic surfactant such as polysorbate (e.g., polysorbate 80; e.g., about 0.15% (w/v)). In an embodiment of the invention, the pharmaceutical formulation is aqueous (e.g., suitable for intravenous and/or subcutaneous administration) and comprises about 200 mg/mL or 274 mg/ml antibody that binds specifically to C5 wherein the antibody comprises a heavy chain immunoglobulin comprising the amino acid sequence:

(SEQ ID NO: 368)
QVQLQESGPGLVKPSETLSLTCTVSGDSVSSSYWTWIRQPPGKGLEWIGY
IYYSGSSNYNPSLKSRATISVDTSKNQFSLKLSSVTAADTAVYYCAREGN
VDTTMIFDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK;

and a light chain immunoglobulin comprising the amino acid sequence:

(SEQ ID NO: 369)
AIQMTQSPSSLSASVGDRVTITCRASQGIRNDLGWYQQKPGKAPKLLIYA
ASSLQSGVPSRFAGRGSGTDFTLTISSLQPEDFATYYCLQDFNYPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC,

about 20 mM histidine (e.g., histidine-HCl), pH about 5.8, about 100 mM L-arginine (e.g., L-arginine HCl or L-arginine monohydrochloride), about 2% (w/v) sucrose, and about 0.15% (w/v) polysorbate 80 (PS-80). In an embodiment of the invention, the formulation includes one or more further therapeutic agents, e.g., an RNA interference agent that binds to an mRNA sequence that encodes C5 partially or fully, for example, comprising an RNA strand comprising the ribonucleotide sequence 5′-UAUUAUAAAAAUAUCUUGCUUUU-3′ (SEQ ID NO: 358); and an RNA strand comprising the ribonucleotide sequence 5′-AAGCAAGAUAUUUUUAUAAUA-3′ (SEQ ID NO: 359). In an embodiment of the invention, the further therapeutic agent is cemdisiran. In an embodiment of the invention, the further therapeutic agent is an anti-coagulant, warfarin, aspirin, heparin, phenindione, fondaparinux, idraparinux, a thrombin inhibitor, argatroban, lepirudin, bivalirudin, dabigatran, an anti-inflammatory drug, a corticosteroid, a non-steroidal anti-inflammatory drug (NSAID), an antihypertensive, an angiotensin-converting enzyme inhibitor, an immunosuppressive agent, vincristine, cyclosporine A, or methotrexate, a fibrinolytic agent ancrod, E-aminocaproic acid, antiplasmin-a1, prostacyclin, defibrotide, a lipid-lowering agent, an inhibitor of hydroxymethylglutaryl CoA reductase, an anti-CD20 agent, rituximab, an anti-TNFalpha agent, infliximab, an anti-seizure agent, magnesium sulfate, a C3 inhibitor and/or an anti-thrombotic agent.

The present invention also provides a method for making a pharmaceutical formulation of the present invention comprising admixing the antigen-binding protein and the carrier components. A pharmaceutical formulation which is a product of such a method also forms part of the present invention.

The present invention also provides a pharmaceutical formulation of the present invention including a vessel or injection device comprising a pharmaceutical formulation of the present invention, e.g., a vial, syringe, pre-filled syringe or autoinjector.

The present invention also provides an intravenous formulation (e.g., a sterile intravenous formulation) comprising a pharmaceutical formulation comprising an anti-C5 antigen-binding protein (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4HI2168P; H4HI2169P; H4H12170P; H4H12171P; H4H12175P; H4H12176P2; H4H12177P2; H4H12183P2; H2M11682N; H2M11684N; H2M11694N; H2M11695N; ravulizumab; eculizumab; crovalimab, tesidolumab or mubodina) and an aqueous intravenous solution (e.g., 0.9% Normal Saline, Lactated Ringers, Dextrose 5% in Water or 0.45% Normal Saline). For example, in an embodiment of the invention, the aqueous intravenous solution has a volume of about 250 ml, 500 ml, 750 ml or 1000 ml. Such an intravenous formulation may include any one or more of NaCl, dextrose, potassium salt, potassium chloride, calcium salt, calcium chloride, sodium lactate and/or lactate salt. A plastic intravenous bag or glass bottle containing an intravenous formulation also forms part of the present invention. Such intravenous formulations can be constituted such that, when administered to a subject, a dose of about 30 mg/kg body weight is achieved. Methods for making such an intravenous formulation comprising the step of introducing a pharmaceutical formulation that is set forth herein into the aqueous intravenous solution are part of the present invention along with intravenous formulations which are products of such a method.

The present invention also provides a method for reducing the viscosity of an aqueous composition that comprises water and about 150 mg/ml or more (e.g., about 200 mg/ml) of anti-C5 antigen-binding protein (for example, about 150 mg/ml, 175 mg/ml, 200 mg/ml, 211 mg/ml, 220 mg/ml, 242 mg/ml or 274 mg/ml, at least about 150 mg/ml, at least about 175 mg/ml, at least about 200 mg/ml, at least about 211 mg/ml, at least about 220 mg/ml, at least about 242 mg/ml or at least about 274 mg/ml, e.g., an anti-C5 antibody or antigen-binding fragment thereof) comprising combining the water and antigen-binding protein with arginine (e.g., 50 mM or 100 mM), and, optionally, one or more additional carrier components (e.g., buffer, non-ionic detergent and/or oligosaccharide). In an embodiment of the invention, the antigen-binding protein is H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4HI2168P; H4HI2169P; H4H12170P; H4H12171P; H4H12175P; H4H12176P2; H4H12177P2; H4H12183P2; H2M11682N; H2M11684N; H2M11694N; H2M11695N; ravulizumab; eculizumab; crovalimab, tesidolumab or mubodina. In an embodiment of the invention, the formulation viscosity is reduced by about 30% or about 30-42%, e.g., wherein viscosity is in units of cP as measured at 20° C.

The present invention also provides a method for administering a pharmaceutical formulation of the present invention to a subject (e.g., a human) comprising introducing (e.g., parenterally, e.g., intravenously, intramuscularly or subcutaneously) the formulation into the body of the subject (e.g., wherein the subject suffers from a C5-associated disease).

The present invention also provides a method for treating or preventing a C5-associated disease (e.g., atypical hemolytic uremic syndrome (aHUS), paroxysmal nocturnal hemoglobinuria (PNH) or CHAPLE disease) in a subject (e.g., a human) in need thereof comprising administering a therapeutically effective amount of antigen-binding protein (e.g., antibody or antigen-binding fragment thereof) that binds specifically to C5 (e.g., human C5) in a pharmaceutical formulation of the present invention to the subject. In an embodiment of the invention, the C5-associated disease is one or more selected from the group consisting of adult respiratory distress syndrome; age-related macular degeneration (AMD); allergy; Alport's syndrome; Alzheimer's disease; antiphospholipid syndrome (APS); asthma; atherosclerosis; atypical hemolytic uremic syndrome (aHUS); autoimmune disease; autoimmune hemolytic anemia (AIHA); balloon angioplasty; bronchoconstriction; bullous pemphigoid; burns; C3 glomerulopathy; capillary leak syndrome; cardiovascular disorder; catastrophic antiphospholipid syndrome (CAPS); cerebrovascular disorder; CHAPLE disease; chemical injury; chronic obstructive pulmonary disease (COPD); cold agglutinin disease (CAD); corneal and/or retinal tissue; Crohn's disease; Degos disease; dense deposit disease (DDD); dermatomyositis; diabetes; diabetic angiopathy; diabetic macular edema (DME); diabetic nephropathy; diabetic retinopathy; dilated cardiomyopathy; disorder of inappropriate or undesirable complement activation; dyspnea; emphysema; epidermolysis bullosa; epilepsy; fibrogenic dust disease; frostbite; geographic atrophy (GA); glomerulonephritis; glomerulopathy; Goodpasture's Syndrome; Graves' disease; Guillain Barre Syndrome; Hashimoto's thyroiditis; hemodialysis complications; hemolysis-elevated liver enzymes—and low platelets (HELLP) syndrome; hemolytic anemia; hemoptysis; Henoch-Schonlein purpura nephritis; hereditary angioedema; hyperacute allograft rejection; hypersensitivity pneumonitis; idiopathic thrombocytopenic purpura (ITP); IgA nephropathy; immune complex disorder; immune complex vasculitis; immune complex-associated inflammation; infectious disease; inflammation caused by an autoimmune disease; inflammatory disorder; inherited CD59 deficiency; injury due to inert dusts and/or minerals; interleukin-2 induced toxicity during IL-2 therapy; ischemia-reperfusion injury; Kawasaki's disease; lung disease or disorder; lupus nephritis; membrane proliferative glomerulonephritis; membrano-proliferative nephritis; mesenteric artery reperfusion after aortic reconstruction; mesenteric/enteric vascular disorder; multifocal motor neuropathy (MMN); multiple sclerosis; myasthenia gravis; myocardial infarction; myocarditis; neurological disorder; neuromyelitis optica; obesity; ocular angiogenesis; ocular neovascularization affecting choroidal; organic dust disease; parasitic disease; Parkinson's disease; paroxysmal nocturnal hemoglobinuria (PNH); Pauci-immune vasculitis; pemphigus; percutaneous transluminal coronary angioplasty (PTCA); peripheral vascular disorder; pneumonia; post-ischemic reperfusion condition; post-pump syndrome in cardiopulmonary bypass; post-pump syndrome in renal bypass; progressive kidney failure; proliferative nephritis; proteinuric kidney disease; psoriasis; pulmonary embolism; pulmonary fibrosis; pulmonary infarction; pulmonary vasculitis; recurrent fetal loss; renal disorder; renal ischemia; renal ischemia-reperfusion injury; renovascular disorder; restenosis following stent placement; rheumatoid arthritis; rotational atherectomy; schizophrenia; sepsis; septic shock; SLE nephritis; smoke injury; spinal cord injury; spontaneous fetal loss; stroke; systemic inflammatory response to sepsis; systemic lupus erythematosus (SLE); systemic lupus erythematosus-associated vasculitis; Takayasu's disease; thermal injury; thrombotic thrombocytopenic purpura (TTP); traumatic brain injury; type I diabetes; typical hemolytic uremic syndrome; uveitis; vasculitis; vasculitis associated with rheumatoid arthritis; venous gas embolus (VGE); and xenograft rejection.

The present invention also provides a method for reducing complement activity in the body of a subject (e.g., a human) in need thereof comprising administering a therapeutically effective amount anti-C5 antigen-binding protein (e.g., antibody or antigen-binding fragment thereof) in a pharmaceutical formulation of the present invention to the subject.

### BRIEF DESCRIPTION OF THE FIGURES

FIG. 1. DSC thermogram of 1 mg/mL H4H12166P determined by VP-DSC. The Tₘ2 shown in the inset represents the slight inflection observed at the beginning of the larger endotherm. This is not a well-defined endotherm and therefore only two Tₘs are reported for this profile in Table 1-3, as represented by the two major endotherms.

FIG. 2. DSC thermogram of 150 mg/mL H4H12166P determined by TA-DSC.

FIG. 3. DSC thermogram of 200 mg/mL formulated H4H12166P determined by TA-DSC.

FIGS. 4A-4B. FIG. 4A provides graphs showing impact of pH, temperature and container headspace on various quality attributes (Δ % high molecular weight (HMW) species; Δ % native species; Δ % low molecular weight (LMW) species; Δ % acidic species; Δ % main species; Δ % basic species; Δ protein concentration; Δ pH; and Δ optical density). FIG. 4B provides graphs showing impact of pH, temperature and container headspace on protein concentration of 150 mg/mL H4H12166P.

FIGS. 5A, 5B, 5C. FIG. 5A provides a graph showing rates of formation of molecular size variants (HMW species) in 150 mg/mL H4H12166P, 20 mM histidine, pH 5.8 at various temperatures; FIG. 5B provides a graph showing rates of formation of molecular size variants (main monomer species) in 150 mg/mL H4H12166P, 20 mM histidine, pH 5.8 at various temperatures; and FIG. 5C provides a graph showing rates of formation of molecular size variants (LMW species) in 150 mg/mL H4H12166P, 20 mM histidine, pH 5.8 at various temperatures. Transfer functions from the DoE study (SE-UPLC results shown in FIG. 4) were used to estimate the rates as a function of incubation temperature.

FIGS. 6A, 6B, 6C. FIG. 6A provides a graph showing rates of formation of charge variants (acidic species) in 150 mg/mL H4H12166P, 20 mM histidine, pH 5.8 at various temperatures; FIG. 6B provides a graph showing rates of formation of charge variants (main peak) in 150 mg/mL H4H12166P, 20 mM histidine, pH 5.8 at various temperatures; FIG. 6C provides a graph showing rates of formation of charge variants (basic species) in 150 mg/mL H4H12166P, 20 mM histidine, pH 5.8 at various temperatures. Transfer functions from the DoE study (CEX-UPLC results shown in FIG. 4) were used to estimate the rates as a function of incubation temperature.

FIG. 7. High molecular weight (HMW) species formed in 150 mg/mL H4H12166P following agitation and freeze/thaw (F/T) stress.

FIG. 8. Acidic charge variant species in 150 mg/mL H4H12166P following agitation and freeze/thaw (F/T) stress.

FIG. 9. Relative % peak areas of oxidized species in 150 mg/mL H4H12166P, determined by HIC-HPLC, following incubation with 500 ppm H₂O₂ at 37° C. for up to 24 hours.

FIG. 10. Total % oxidation levels in 150 mg/mL H4H12166P determined by HIC-HPLC following forced oxidation with different concentrations of hydrogen peroxide at 37° C. for up to 24 hours.

FIG. 11. Acidic charge variants in 150 mg/mL H4H12166P determined by CEX-UPLC following incubation with different concentrations of hydrogen peroxide at 37° C. for up to 24 hours.

FIG. 12. Basic charge variants in 150 mg/mL H4H12166P determined by CEX-UPLC following incubation with different concentrations of hydrogen peroxide at 37° C. for up to 24 hours.

FIG. 13. High molecular weight (HMW) species in 150 mg/mL H4H12166P determined by SE-UPLC following incubation with different concentrations of hydrogen peroxide at 37° C. for up to 24 hours.

FIG. 14. Exemplary formulations of the present disclosure.

### DETAILED DESCRIPTION

The pharmaceutical formulations of the present invention are characterized by a number of particularly advantageous properties. The formulations have both high protein concentration and low viscosity. The particularly low viscosity of the formulations contrast with that of several commercially available anti-C5 antibody products. The low viscosity of the formulations of the present invention facilitate the delivery of a large amount of anti-C5 antibody in a low volume. Moreover, the pharmaceutical formulations of the present invention exhibit a high degree of stability-resistance to meaningful increases in high molecular weight (HMW) species under highly oxidizing conditions and only minimal increases in HMW species after several hours of agitation.

A “high molecular weight” (HMW) species as used herein, for example, with reference to a pharmaceutical formulation containing a given anti-C5 antibody or antigen-binding fragment thereof, refers to any species of antibody or antigen-binding fragment thereof in the formulation which elutes from a size exclusion column (e.g., SE-UPLC) ahead of (e.g., with a higher molecular weight than) that of a single species of such antibody (a tetrameric complex with two heavy and two light chains) or an antigen-binding fragment thereof. The percentage of HMW species refers to the percentage of such species relative to the overall quantity of antibody or antigen-binding fragment thereof in the formulation, e.g., by SE-UPLC analysis.

A “low molecular weight” (LMW) species as used herein, for example, with reference to a pharmaceutical formulation containing a given anti-C5 antibody or antigen-binding fragment thereof, refers to any species of antibody or antigen-binding fragment thereof in the formulation which elutes from a size exclusion column (e.g., SE-UPLC) behind (e.g., with a lower molecular weight than) that of a single species of such antibody (a tetrameric complex with two heavy and two light chains) or an antigen-binding fragment thereof. The percentage of LMW species refers to the percentage of such species relative to the overall quantity of antibody or antigen-binding fragment thereof in the formulation, e.g., by SE-UPLC analysis.

Concentrations of the excipients in the formulations of the present invention may be expressed in percentages (%) which are weight/volume (w/v) units. Weight/volume refers to the mass of a component/volume of solution×100.

The term “C5”, also called “complement component 5” or “complement factor 5” refers to the serum protein of the complement cascade. The C5 protein is a 1676 amino acid protein comprising two chains, alpha and beta. The protein represents the convergence point for three complement activation pathways: classical pathway, alternative pathway and the mannose binding lectin pathway. The amino acid sequence of full-length C5 protein is exemplified by the amino acid sequence provided in GenBank as accession number NP001726.2.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

### Anti-C5 Antigen-Binding Proteins

The present invention provides pharmaceutical formulations comprising anti-C5 antigen-binding proteins (e.g., antibodies and antigen-binding fragments thereof) and a pharmaceutically acceptable carrier.

In an embodiment of the invention, the anti-C5 antigen-binding protein binds to the beta chain or the alpha chain of C5 or both, e.g., at residues 591-599 and/or 775-794, e.g., NMATGMDSW (SEQ ID NO: 353) and/or WEVHLVPRRKQLQFALPDSL (SEQ ID NO: 354). In an embodiment of the invention, the anti-C5 antigen-binding protein does not bind C5a.

In an embodiment of the invention, the anti-C5 antigen-binding protein binds C5 at residues KDMQLGRLHMKTLLPVSK (SEQ ID NO: 355).

In an embodiment of the invention, the anti-C5 antigen-binding protein binds the beta chain of C5 thereof, e.g., at residues 332-398, 332-378, 332-364, 332-348, 350-420, 369-409, 379-398 and/or 386-392.

In an embodiment of the invention, the anti-C5 antigen-binding protein binds C5a, e.g., at residues NDETCEQRA (SEQ ID NO: 356) and/or SHKDMQL (SEQ ID NO: 357).

In an embodiment of the invention, the anti-C5 antigen-binding protein binds the beta chain of C5, e.g., residues 19-180. In an embodiment of the invention, binding to C5 is reduced by E48A, D51A and/or K109A C5 mutations.

Immunoglobulin polypeptides in anti-C5 antigen binding proteins (e.g., antibody or antigen-binding fragment thereof) of the pharmaceutical formulations of the present invention are set forth in Table A. See International patent application publication no. WO2017/218515.

TABLE A
Anti-C5 Antibody Chain Amino Acid Sequences*

| Antibody | SEQ ID NOs | | | | | | | |
|---|---|---|---|---|---|---|---|---|
| designation | HCVR | HCDR1 | HCDR2 | HCDR3 | LCVR | LCDR1 | LCDR2 | LCDR3 |
| H2M11683N | 2 | 4 | 6 | 8 | 10 | 12 | 14 | 16 |
| H2M11686N | 18 | 20 | 22 | 24 | 26 | 28 | 30 | 32 |
| H4H12159P | 34 | 36 | 38 | 40 | 42 | 44 | 46 | 48 |
| H4H12161P | 50 | 52 | 54 | 56 | 58 | 60 | 62 | 64 |
| H4H12163P | 66 | 68 | 70 | 72 | 74 | 76 | 78 | 80 |
| H4H12164P | 82 | 84 | 86 | 88 | 90 | 92 | 94 | 96 |
| H4H12166P | 98 | 100 | 102 | 104 | 106 | 108 | 110 | 112 |
| H4H12166P2 | 98 | 100 | 102 | 104 | 114 | 116 | 118 | 120 |
| H4H12166P3 | 122 | 124 | 126 | 128 | 106 | 108 | 110 | 112 |
| H4H12166P4 | 98 | 100 | 102 | 104 | 130 | 132 | 134 | 136 |
| H4H12166P5 | 138 | 140 | 142 | 144 | 106 | 108 | 110 | 112 |
| H4H12166P6 | 146 | 148 | 150 | 152 | 106 | 108 | 110 | 112 |
| H4H12166P7 | 122 | 124 | 126 | 128 | 130 | 132 | 134 | 136 |
| H4H12166P8 | 146 | 148 | 150 | 152 | 114 | 116 | 118 | 120 |
| H4H12166P9 | 146 | 148 | 150 | 152 | 130 | 132 | 134 | 136 |
| H4H12166P10 | 138 | 140 | 142 | 144 | 130 | 132 | 134 | 136 |
| H4H12167P | 154 | 156 | 158 | 160 | 162 | 164 | 166 | 168 |
| H4H12168P | 170 | 172 | 174 | 176 | 178 | 180 | 182 | 184 |
| H4H12169P | 186 | 188 | 190 | 192 | 194 | 196 | 198 | 200 |
| H4H12170P | 202 | 204 | 206 | 208 | 210 | 212 | 214 | 216 |
| H4H12171P | 218 | 220 | 222 | 224 | 226 | 228 | 230 | 232 |
| H4H12175P | 234 | 236 | 238 | 240 | 242 | 244 | 246 | 248 |
| H4H12176P2 | 250 | 252 | 254 | 256 | 258 | 260 | 262 | 264 |
| H4H12177P2 | 266 | 268 | 270 | 272 | 258 | 260 | 262 | 264 |
| H4H12183P2 | 274 | 276 | 278 | 280 | 282 | 284 | 286 | 288 |
| H2M11682N | 290 | 292 | 294 | 296 | 298 | 300 | 302 | 304 |
| H2M11684N | 306 | 308 | 310 | 312 | 314 | 316 | 318 | 320 |
| H2M11694N | 322 | 324 | 326 | 328 | 330 | 332 | 334 | 336 |
| H2M11695N | 338 | 340 | 342 | 344 | 346 | 348 | 350 | 352 |

*Antibodies and fragments may include one or more variants of said sequences

TABLE B
Anti-C5 Antibody Chain Nucleotide Sequences*

| Antibody | SEQ ID NOs | | | | | | | |
|---|---|---|---|---|---|---|---|---|
| designation | HCVR | HCDR1 | HCDR2 | HCDR3 | LCVR | LCDR1 | LCDR2 | LCDR3 |
| H2M11683N | 1 | 3 | 5 | 7 | 9 | 11 | 13 | 15 |
| H2M11686N | 17 | 19 | 21 | 23 | 25 | 27 | 29 | 31 |
| H4H12159P | 33 | 35 | 37 | 39 | 41 | 43 | 45 | 47 |
| H4H12161P | 49 | 51 | 53 | 55 | 57 | 59 | 61 | 63 |
| H4H12163P | 65 | 67 | 69 | 71 | 73 | 75 | 77 | 79 |
| H4H12164P | 81 | 83 | 85 | 87 | 89 | 91 | 93 | 95 |
| H4H12166P | 97 | 99 | 101 | 103 | 105 | 107 | 109 | 111 |
| H4H12166P2 | 97 | 99 | 101 | 103 | 113 | 115 | 117 | 119 |
| H4H12166P3 | 121 | 123 | 125 | 127 | 105 | 107 | 109 | 111 |
| H4H12166P4 | 97 | 99 | 101 | 103 | 129 | 131 | 133 | 135 |
| H4H12166P5 | 137 | 139 | 141 | 143 | 105 | 107 | 109 | 111 |
| H4H12166P6 | 145 | 147 | 149 | 151 | 105 | 107 | 109 | 111 |
| H4H12166P7 | 121 | 123 | 125 | 127 | 129 | 131 | 133 | 135 |
| H4H12166P8 | 145 | 147 | 149 | 151 | 113 | 115 | 117 | 119 |
| H4H12166P9 | 145 | 147 | 149 | 151 | 129 | 131 | 133 | 135 |
| H4H12166P10 | 137 | 139 | 141 | 143 | 129 | 131 | 133 | 135 |
| H4H12167P | 153 | 155 | 157 | 159 | 161 | 163 | 165 | 167 |
| H4H12168P | 169 | 171 | 173 | 175 | 177 | 179 | 181 | 183 |
| H4H12169P | 185 | 187 | 189 | 191 | 193 | 195 | 197 | 199 |
| H4H12170P | 201 | 203 | 205 | 207 | 209 | 211 | 213 | 215 |
| H4H12171P | 217 | 219 | 221 | 223 | 225 | 227 | 229 | 231 |
| H4H12175P | 233 | 235 | 237 | 239 | 241 | 243 | 245 | 247 |
| H4H12176P2 | 249 | 251 | 253 | 255 | 257 | 259 | 261 | 263 |
| H4H12177P2 | 265 | 267 | 269 | 271 | 257 | 259 | 261 | 263 |
| H4H12183P2 | 273 | 275 | 277 | 279 | 281 | 283 | 285 | 287 |
| H2M11682N | 289 | 291 | 293 | 295 | 297 | 299 | 301 | 303 |
| H2M11684N | 305 | 307 | 309 | 311 | 313 | 315 | 317 | 319 |
| H2M11694N | 321 | 323 | 325 | 327 | 329 | 331 | 333 | 335 |
| H2M11695N | 337 | 339 | 341 | 343 | 345 | 347 | 349 | 351 |

*Antibodies and fragments may include one or more variants of said sequences

In an embodiment of the invention, the anti-C5 antigen-binding protein is eculizumab (sold as Soliris), crovalimab, ravulizumab (ALXN1210; sold as Ultomiris), tesidolumab (see U.S. Pat. No. 8,241,628; WO 2010/015608; or WO2017/212375) or mubodina (see U.S. Pat. No. 7,999,081). In an embodiment of the invention, the anti-C5 antigen-binding protein is pozelimab (REGN3918; H4H12166P) antibody. Pozelimab (REGN3918; H4H12166P) antibody comprises a heavy chain immunoglobulin comprising the amino acid sequence:

(SEQ ID NO: 368)
QVQLQESGPG LVKPSETLSL TCTVSGDSVS SSYWTWIRQP
PGKGLEWIGY IYYSGSSNYN PSLKSRATIS VDTSKNQFSL
KLSSVTAADT AVYYCAREGN VDTTMIFDYW GQGTLVTVSS
ASTKGPSVFP LAPCSRSTSE STAALGCLVK DYFPEPVTVS
WNSGALTSGV HTFPAVLQSS GLYSLSSVVT VPSSSLGTKT
YTCNVDHKPS NTKVDKRVES KYGPPCPPCP APEFLGGPSV
FLFPPKPKDT LMISRTPEVT CVVVDVSQED PEVQFNWYVD
GVEVHNAKTK PREEQFNSTY RVVSVLTVLH QDWLNGKEYK
CKVSNKGLPS SIEKTISKAK GQPREPQVYT LPPSQEEMTK
NQVSLTCLVK GFYPSDIAVE WESNGQPENN YKTTPPVLDS
DGSFFLYSRL TVDKSRWQEG NVFSCSVMHE ALHNHYTQKS
LSLSLGK;

and a light chain immunoglobulin comprising the amino acid sequence:

(SEQ ID NO: 369)
AIQMTQSPSS LSASVGDRVT ITCRASQGIR NDLGWYQQKP
GKAPKLLIYA ASSLQSGVPS RFAGRGSGTD FTLTISSLQP
EDFATYYCLQ DFNYPWTFGQ GTKVEIKRTV AAPSVFIFPP
SDEQLKSGTA SVVCLLNNFY PREAKVQWKV DNALQSGNSQ
ESVTEQDSKD STYSLSSTLT LSKADYEKHK VYACEVTHQG
LSSPVTKSFN RGEC.

### See WO2017/218515.

In an embodiment of the invention, the anti-C5 antigen-binding protein comprises a heavy chain immunoglobulin comprising the amino acid sequence:

(SEQ ID NO: 370)
QVQLVESGGGLVQPGRSLRLSCAASGFTVHSSYYMAWVRQAPGKGLEWVG
AIFTGSGAEYKAEWAKGRVTISKDTSKNQVVLTMTNMDPVDTATYYCASD
AGYDYPTHAMHYWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELRRGPKVFLFP
PKPKDTLMISRTPEVTCVVVDVSHEDPEVKFNWYVDGVEVHNAKTKPREE
QYNSTYRVVSVLTVLHQDWLNGKEYKCKVSNKGLPSSIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVLHEALHAHYTRKELSLS
P

or the HCDR1, HCDR2 and HCDR3 thereof; or the V_{H} thereof (or a variant thereof);
and a light chain immunoglobulin comprising the amino acid sequence:

(SEQ ID NO: 371)
DIQMTQSPSSLSASVGDRVTITCRASQGISSSLAWYQQKPGKAPKLLIYG
ASETESGVPSRFSGSGSGTDFTLTISSLQPEDFATYYCQNTKVGSSYGNT
FGGGTKVEIKRTVAAPSVFIFTPSDEQLKSGTASVVCLLNNFYPREAKVQ
WKVDNALQSGNSQESVTEQDSKDSTYSLSSTLTLSKADYEKHKVYACEVT
HQGLSSPVTKSFNRGEC

or the LCDR1, LCDR2 and LCDR3 thereof; or the V_{L} thereof (or a variant thereof).

The present invention includes pharmaceutical formulations comprising antibodies and antigen-binding fragments thereof that include the variable regions and CDRs which are specifically discussed herein as well as variable regions and CDRs which are variants of those discussed herein.

A “variant” of a polypeptide, such as an immunoglobulin chain (e.g., the H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4HI2168P; H4HI2169P; H4H12170P; H4H12171P; H4H12175P; H4H12176P2; H4H12177P2; H4H12183P2; H2M11682N; H2M11684N; H2M11694N; H2M11695N; ravulizumab, eculizumab, crovalimab, tesidolumab or mubodina V_{H}, V_{L}, HC or LC or CDR thereof comprising the amino acid sequence specifically set forth herein), refers to a polypeptide comprising an amino acid sequence that is at least about 70-99.9% (e.g., at least 70, 72, 74, 75, 76, 79, 80, 81, 82, 83, 84, 85, 86, 87, 88, 89, 90, 91, 92, 93, 94, 95, 96, 97, 98, 99, 99.5 or 99.9%) identical or similar to a referenced amino acid sequence that is set forth herein (e.g., any of SEQ ID NOs: 2; 4; 6; 8; 10; 12; 14; 16; 18; 20; 22; 24; 26; 28; 30; 32; 34; 36; 38; 40; 42; 44; 46; 48; 50; 52; 54; 56; 58; 60; 62; 64; 66; 68; 70; 72; 74; 76; 78; 80; 82; 84; 86; 88; 90; 92; 94; 96; 98; 98; 98; 100; 100; 100; 102; 102; 102; 104; 104; 104; 106; 106; 106; 106; 108; 108; 108; 108; 110; 110; 110; 110; 112; 112; 112; 112; 114; 114; 116; 116; 118; 118; 120; 120; 122; 122; 124; 124; 126; 126; 128; 128; 130; 130; 130; 130; 132; 132; 132; 132; 134; 134; 134; 134; 136; 136; 136; 136; 138; 138; 140; 140; 142; 142; 144; 144; 146; 146; 146; 148; 148; 148; 150; 150; 150; 152; 152; 152; 154; 156; 158; 160; 162; 164; 166; 168; 170; 172; 174; 176; 178; 180; 182; 184; 186; 188; 190; 192; 194; 196; 198; 200; 202; 204; 206; 208; 210; 212; 214; 216; 218; 220; 222; 224; 226; 228; 230; 232; 234; 236; 238; 240; 242; 244; 246; 248; 250; 252; 254; 256; 258; 258; 260; 260; 262; 262; 264; 264; 266; 268; 270; 272; 274; 276; 278; 280; 282; 284; 286; 288; 290; 292; 294; 296; 298; 300; 302; 304; 306; 308; 310; 312; 314; 316; 318; 320; 322; 324; 326; 328; 330; 332; 334; 336; 338; 340; 342; 344; 346; 348; 350, 352, 353, 354, 355, 356, 357, 362, 363, 364, 365, 366, 367, 368 and/or 369), see e.g., Table A; when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment).

Moreover, a variant of a polypeptide may include a polypeptide such as an immunoglobulin chain (e.g., the H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4HI2168P; H4HI2169P; H4H12170P; H4H12171P; H4H12175P; H4H12176P2; H4H12177P2; H4H12183P2; H2M11682N; H2M11684N; H2M11694N; H2M11695N; ravulizumab, eculizumab, crovalimab, tesidolumab or mubodina V_{H}, V_{L}, HC or LC or CDR thereof) may include the amino acid sequence of the reference polypeptide whose amino acid sequence is specifically set forth herein but for one or more (e.g., 1, 2, 3, 4, 5, 6, 7, 8, 9 or 10) mutations, e.g., one or more missense mutations (e.g., conservative substitutions), non-sense mutations, deletions, or insertions. For example, the present invention includes pharmaceutical formulations including one or more anti-C5 antigen-binding proteins which include an immunoglobulin light chain (or V_{L}) variant comprising the amino acid sequence set forth in SEQ ID NO: 106 but having one or more of such mutations and/or an immunoglobulin heavy chain (or V_{H}) variant comprising the amino acid sequence set forth in SEQ ID NO: 98 but having one or more of such mutations. In an embodiment of the invention, the anti-C5 antigen-binding protein includes an immunoglobulin light chain variant comprising CDR-L1, CDR-L2 and CDR-L3 wherein one or more (e.g., 1 or 2 or 3) of such CDRs has one or more of such mutations (e.g., conservative substitutions) and/or an immunoglobulin heavy chain variant comprising CDR-H1, CDR-H2 and CDR-H3 wherein one or more (e.g., 1 or 2 or 3) of such CDRs has one or more of such mutations (e.g., conservative substitutions).

The following references relate to BLAST algorithms often used for sequence analysis: BLAST ALGORITHMS: Altschul et al. (2005) FEBS J. 272(20): 5101-5109; Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

“H2M11683N”; “H2M11686N”; “H4H12159P”; “H4H12161P”; “H4H12163P”; “H4H12164P”; “H4H12166P”; “H4H12166P2”; “H4H12166P3”; “H4H12166P4”; “H4H12166P5”; “H4H12166P6”; “H4H12166P7”; “H4H12166P8”; “H4H12166P9”; “H4H12166P10”; “H4H12167P”; “H4HI2168P”; “H4HI2169P”; “H4H12170P”; “H4H12171P”; “H4H12175P”; “H4H12176P2”; “H4H12177P2”; “H4H12183P2”; “H2M11682N”; “H2M11684N”; “H2M11694N” or “H2M11695N”, unless otherwise stated, refer to anti-C5 antigen-binding proteins, e.g., antibodies and antigen-binding fragments thereof (including multispecific antigen-binding proteins), that bind specifically to C5, comprising an immunoglobulin heavy chain or variable region thereof (V_{H}) comprising the amino acid sequence specifically set forth herein corresponding, in Table A herein or Table 1 of WO2017/218515 (and the sequences set forth therein), to H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4HI2168P; H4HI2169P; H4H12170P; H4H12171P; H4H12175P; H4H12176P2; H4H12177P2; H4H12183P2; H2M11682N; H2M11684N; H2M11694N; or H2M11695N (e.g., SEQ ID NO: 2; 18; 34; 50; 66; 82; 98; 98; 122; 98; 138; 146; 122; 146; 146; 138; 154; 170; 186; 202; 218; 234; 250; 266; 274; 290; 306; 322 or 338) (or a variant thereof), and/or an immunoglobulin light chain or variable region thereof (V_{L}) comprising the amino acid sequence specifically set forth herein corresponding, in Table A herein or Table 1 of WO2017/218515 (and the sequences set forth therein), to H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4HI2168P; H4HI2169P; H4H12170P; H4H12171P; H4H12175P; H4H12176P2; H4H12177P2; H4H12183P2; H2M11682N; H2M11684N; H2M11694N or H2M11695N (e.g., SEQ ID NO: 10; 26; 42; 58; 74; 90; 106; 114; 106; 130; 106; 106; 130; 114; 130; 130; 162; 178; 194; 210; 226; 242; 258; 258; 282; 298; 314; 330 or 346) (or a variant thereof), respectively; and/or that comprise a heavy chain or V_{H} that comprises the CDRs thereof (CDR-H1 (or a variant thereof), CDR-H2 (or a variant thereof) and CDR-H3 (or a variant thereof)) and/or a light chain or V_{L} that comprises the CDRs thereof (CDR-L1 (or a variant thereof), CDR-L2 (or a variant thereof) and CDR-L3 (or a variant thereof)). In an embodiment of the invention, the V_{H} is linked to an IgG constant heavy chain domain (e.g., IgG1 or IgG4 (e.g., IgG4 (S228P mutant))) and/or the V_{L} is linked to a lambda or kappa constant light chain domain.

In an embodiment of the invention, the antigen-binding protein, H2M11683N, comprises an HCVR that comprises the amino acid sequence set forth in SEQ ID NO: 2 and an LCVR that comprises the amino acid sequence set forth in SEQ ID NO: 10 (e.g., wherein the antigen-binding protein is an antibody or antigen-binding fragment thereof).

In an embodiment of the invention, the antigen-binding protein, H2M11686N, comprises an HCVR that comprises the amino acid sequence set forth in SEQ ID NO: 18 and an LCVR that comprises the amino acid sequence set forth in SEQ ID NO: 26 (e.g., wherein the antigen-binding protein is an antibody or antigen-binding fragment thereof).

In an embodiment of the invention, the antigen-binding protein, H4H12159P, comprises an HCVR that comprises the amino acid sequence set forth in SEQ ID NO: 34 and an LCVR that comprises the amino acid sequence set forth in SEQ ID NO: 42 (e.g., wherein the antigen-binding protein is an antibody or antigen-binding fragment thereof).

In an embodiment of the invention, the antigen-binding protein, H4H12161P, comprises an HCVR that comprises the amino acid sequence set forth in SEQ ID NO: 50 and an LCVR that comprises the amino acid sequence set forth in SEQ ID NO: 58 (e.g., wherein the antigen-binding protein is an antibody or antigen-binding fragment thereof).

In an embodiment of the invention, the antigen-binding protein, H4H12163P, comprises an HCVR that comprises the amino acid sequence set forth in SEQ ID NO: 66 and an LCVR that comprises the amino acid sequence set forth in SEQ ID NO: 74 (e.g., wherein the antigen-binding protein is an antibody or antigen-binding fragment thereof).

In an embodiment of the invention, the antigen-binding protein, H4H12164P, comprises an HCVR that comprises the amino acid sequence set forth in SEQ ID NO: 82 and an LCVR that comprises the amino acid sequence set forth in SEQ ID NO: 90 (e.g., wherein the antigen-binding protein is an antibody or antigen-binding fragment thereof).

In an embodiment of the invention, the antigen-binding protein, H4H12166P, comprises an HCVR that comprises the amino acid sequence set forth in SEQ ID NO: 98 and an LCVR that comprises the amino acid sequence set forth in SEQ ID NO: 106 (e.g., wherein the antigen-binding protein is an antibody or antigen-binding fragment thereof).

In an embodiment of the invention, the antigen-binding protein, H4H12166P2, comprises an HCVR that comprises the amino acid sequence set forth in SEQ ID NO: 98 and an LCVR that comprises the amino acid sequence set forth in SEQ ID NO: 114 (e.g., wherein the antigen-binding protein is an antibody or antigen-binding fragment thereof).

In an embodiment of the invention, the antigen-binding protein, H4H12166P3, comprises an HCVR that comprises the amino acid sequence set forth in SEQ ID NO: 122 and an LCVR that comprises the amino acid sequence set forth in SEQ ID NO: 106 (e.g., wherein the antigen-binding protein is an antibody or antigen-binding fragment thereof).

In an embodiment of the invention, the antigen-binding protein, H4H12166P4, comprises an HCVR that comprises the amino acid sequence set forth in SEQ ID NO: 98 and an LCVR that comprises the amino acid sequence set forth in SEQ ID NO: 130 (e.g., wherein the antigen-binding protein is an antibody or antigen-binding fragment thereof).

In an embodiment of the invention, the antigen-binding protein, H4H12166P5, comprises an HCVR that comprises the amino acid sequence set forth in SEQ ID NO: 138 and an LCVR that comprises the amino acid sequence set forth in SEQ ID NO: 106 (e.g., wherein the antigen-binding protein is an antibody or antigen-binding fragment thereof).

In an embodiment of the invention, the antigen-binding protein, H4H12166P6, comprises an HCVR that comprises the amino acid sequence set forth in SEQ ID NO: 146 and an LCVR that comprises the amino acid sequence set forth in SEQ ID NO: 106 (e.g., wherein the antigen-binding protein is an antibody or antigen-binding fragment thereof).

In an embodiment of the invention, the antigen-binding protein, H4H12166P7, comprises an HCVR that comprises the amino acid sequence set forth in SEQ ID NO: 122 and an LCVR that comprises the amino acid sequence set forth in SEQ ID NO: 130 (e.g., wherein the antigen-binding protein is an antibody or antigen-binding fragment thereof).

In an embodiment of the invention, the antigen-binding protein, H4H12166P8, comprises an HCVR that comprises the amino acid sequence set forth in SEQ ID NO: 146 and an LCVR that comprises the amino acid sequence set forth in SEQ ID NO: 114 (e.g., wherein the antigen-binding protein is an antibody or antigen-binding fragment thereof).

In an embodiment of the invention, the antigen-binding protein, H4H12166P9, comprises an HCVR that comprises the amino acid sequence set forth in SEQ ID NO: 146 and an LCVR that comprises the amino acid sequence set forth in SEQ ID NO: 130 (e.g., wherein the antigen-binding protein is an antibody or antigen-binding fragment thereof).

In an embodiment of the invention, the antigen-binding protein, H4H12166P10, comprises an HCVR that comprises the amino acid sequence set forth in SEQ ID NO: 138 and an LCVR that comprises the amino acid sequence set forth in SEQ ID NO: 130 (e.g., wherein the antigen-binding protein is an antibody or antigen-binding fragment thereof).

In an embodiment of the invention, the antigen-binding protein, H4H12167P, comprises an HCVR that comprises the amino acid sequence set forth in SEQ ID NO: 154 and an LCVR that comprises the amino acid sequence set forth in SEQ ID NO: 162 (e.g., wherein the antigen-binding protein is an antibody or antigen-binding fragment thereof).

In an embodiment of the invention, the antigen-binding protein, H4H12168P, comprises an HCVR that comprises the amino acid sequence set forth in SEQ ID NO: 170 and an LCVR that comprises the amino acid sequence set forth in SEQ ID NO: 178 (e.g., wherein the antigen-binding protein is an antibody or antigen-binding fragment thereof).

In an embodiment of the invention, the antigen-binding protein, H4H12169P, comprises an HCVR that comprises the amino acid sequence set forth in SEQ ID NO: 186 and an LCVR that comprises the amino acid sequence set forth in SEQ ID NO: 194 (e.g., wherein the antigen-binding protein is an antibody or antigen-binding fragment thereof).

In an embodiment of the invention, the antigen-binding protein, H4H12170P, comprises an HCVR that comprises the amino acid sequence set forth in SEQ ID NO: 202 and an LCVR that comprises the amino acid sequence set forth in SEQ ID NO: 210 (e.g., wherein the antigen-binding protein is an antibody or antigen-binding fragment thereof).

In an embodiment of the invention, the antigen-binding protein, H4H12171P, comprises an HCVR that comprises the amino acid sequence set forth in SEQ ID NO: 218 and an LCVR that comprises the amino acid sequence set forth in SEQ ID NO: 226 (e.g., wherein the antigen-binding protein is an antibody or antigen-binding fragment thereof).

In an embodiment of the invention, the antigen-binding protein, H4H12175P, comprises an HCVR that comprises the amino acid sequence set forth in SEQ ID NO: 234 and an LCVR that comprises the amino acid sequence set forth in SEQ ID NO: 242 (e.g., wherein the antigen-binding protein is an antibody or antigen-binding fragment thereof).

In an embodiment of the invention, the antigen-binding protein, H4H12176P2, comprises an HCVR that comprises the amino acid sequence set forth in SEQ ID NO: 250 and an LCVR that comprises the amino acid sequence set forth in SEQ ID NO: 258 (e.g., wherein the antigen-binding protein is an antibody or antigen-binding fragment thereof).

In an embodiment of the invention, the antigen-binding protein, H4H12177P2, comprises an HCVR that comprises the amino acid sequence set forth in SEQ ID NO: 266 and an LCVR that comprises the amino acid sequence set forth in SEQ ID NO: 258 (e.g., wherein the antigen-binding protein is an antibody or antigen-binding fragment thereof).

In an embodiment of the invention, the antigen-binding protein, H4H12183P2, comprises an HCVR that comprises the amino acid sequence set forth in SEQ ID NO: 274 and an LCVR that comprises the amino acid sequence set forth in SEQ ID NO: 282 (e.g., wherein the antigen-binding protein is an antibody or antigen-binding fragment thereof).

In an embodiment of the invention, the antigen-binding protein, H2M11682N, comprises an HCVR that comprises the amino acid sequence set forth in SEQ ID NO: 290 and an LCVR that comprises the amino acid sequence set forth in SEQ ID NO: 298 (e.g., wherein the antigen-binding protein is an antibody or antigen-binding fragment thereof).

In an embodiment of the invention, the antigen-binding protein, H2M11684N, comprises an HCVR that comprises the amino acid sequence set forth in SEQ ID NO: 306 and an LCVR that comprises the amino acid sequence set forth in SEQ ID NO: 314 (e.g., wherein the antigen-binding protein is an antibody or antigen-binding fragment thereof).

In an embodiment of the invention, the antigen-binding protein, H2M11694N, comprises an HCVR that comprises the amino acid sequence set forth in SEQ ID NO: 322 and an LCVR that comprises the amino acid sequence set forth in SEQ ID NO: 330 (e.g., wherein the antigen-binding protein is an antibody or antigen-binding fragment thereof).

In an embodiment of the invention, the antigen-binding protein, H2M11695N, comprises an HCVR that comprises the amino acid sequence set forth in SEQ ID NO: 338 and an LCVR that comprises the amino acid sequence set forth in SEQ ID NO: 346 (e.g., wherein the antigen-binding protein is an antibody or antigen-binding fragment thereof).

In an embodiment of the invention, an anti-C5 antigen-binding protein, e.g., antibody or antigen-binding fragment, comprises a heavy chain constant domain, e.g., of the type IgA (e.g., IgA1 or IgA2), IgD, IgE, IgG (e.g., IgG1, IgG2, IgG3 and IgG4 (e.g., comprising a S228P mutation)) or IgM. Silva et al., J Biol Chem. 290(9):5462-9 (2015). In an embodiment of the invention, an antigen-binding protein, e.g., antibody or antigen-binding fragment, comprises a light chain constant domain, e.g., of the type kappa or lambda. The present invention includes pharmaceutical formulations including antigen-binding proteins comprising the variable domains set forth herein and in the art (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4HI2168P; H4HI2169P; H4H12170P; H4H12171P; H4H12175P; H4H12176P2; H4H12177P2; H4H12183P2; H2M11682N; H2M11684N; H2M11694N; H2M11695N; ravulizumab, eculizumab, crovalimab, tesidolumab, mubodina, IFX-1 (see e.g., US2017/0137499), olendalizumab) which are linked to a heavy and/or light chain constant domain, e.g., as set forth above (e.g., an IgG4 heavy chain constant region and a kappa light chain constant region).

The term “antibody”, as used herein, refers to immunoglobulin molecules comprising four polypeptide chains, two heavy chains (HCs) including three H-CDRs and two light chains (LCs) including three L-CDRs inter-connected by disulfide bonds (i.e., “full antibody molecules”) (e.g., IgG4)—for example H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4HI2168P; H4HI2169P; H4H12170P; H4H12171P; H4H12175P; H4H12176P2; H4H12177P2; H4H12183P2; H2M11682N; H2M11684N; H2M11694N; or H2M11695N. In an embodiment of the invention, the assignment of amino acids to each CDR domain within an immunoglobulin chain is in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883. Thus, the present invention includes antibodies and antigen-binding fragments including the CDRs of a V_{H} and the CDRs of a V_{L}, which V_{H} and V_{L} comprise amino acid sequences as set forth herein (or a variant thereof), wherein the CDRs are as defined according to Kabat and/or Chothia.

The terms “antigen-binding portion” or “antigen-binding fragment” of an antibody or antigen-binding protein, and the like, as used herein, include any naturally occurring, enzymatically obtainable, synthetic, or genetically engineered polypeptide or glycoprotein that specifically binds an antigen to form a complex. Non-limiting examples of antigen-binding fragments include: (i) Fab fragments; (ii) F(ab′)₂ fragments; (iii) Fd fragments (heavy chain portion of a Fab fragment cleaved with papain); (iv) Fv fragments (a V_{H} or V_{L}); and (v) single-chain Fv (scFv) molecules; consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies and small modular immunopharmaceuticals (SMIPs), are also encompassed within the expression “antigen-binding fragment,” as used herein. In an embodiment of the invention, the antigen-binding fragment comprises three or more CDRs of H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4HI2168P; H4HI2169P; H4H12170P; H4H12171P; H4H12175P; H4H12176P2; H4H12177P2; H4H12183P2; H2M11682N; H2M11684N; H2M11694N; or H2M11695N (e.g., CDR-H1, CDR-H2 and CDR-H3; and/or CDR-L1, CDR-L2 and CDR-L3).

The term “recombinant” antigen-binding proteins, such as antibodies or antigen-binding fragments thereof, refers to such molecules created, expressed, isolated or obtained by technologies or methods known in the art as recombinant DNA technology which include, e.g., DNA splicing and transgenic expression. The term includes antibodies expressed in a non-human mammal (including transgenic non-human mammals, e.g., transgenic mice), or a host cell (e.g., Chinese hamster ovary (CHO) cell) or cellular expression system or isolated from a recombinant combinatorial human antibody library. The present invention includes recombinant antigen-binding proteins as set forth herein (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4HI2168P; H4HI2169P; H4H12170P; H4H12171P; H4H12175P; H4H12176P2; H4H12177P2; H4H12183P2; H2M11682N; H2M11684N; H2M11694N; or H2M11695N).

The present invention includes formulations comprising monoclonal anti-C5 antigen-binding proteins (e.g., antibodies and antigen-binding fragments thereof). The term “monoclonal antibody” or “mAb”, as used herein, refers to an antibody from a population of substantially homogeneous antibodies, i.e., the antibody molecules comprising the population are identical in amino acid sequence except for possible naturally occurring mutations that may be present in minor amounts. The modifier “monoclonal” is not to be construed as requiring production of the antibody by any particular method. Monoclonal antibodies may be made by the hybridoma method of Kohler et al. (1975) Nature 256: 495, or may be made by recombinant DNA methods (see, e.g., U.S. Pat. No. 4,816,567).

“Isolated” antigen-binding proteins (e.g., antibodies or antigen-binding fragments thereof), polypeptides, polynucleotides and vectors, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antigen-binding protein may further be at least partially free of expression system components such as biological molecules from a host cell or of the growth medium thereof. Generally, the term “isolated” is not intended to be limited to a complete absence of such biological molecules (e.g., minor or insignificant amounts of impurity may remain) or to an absence of water, buffers, or salts or to components of a pharmaceutical formulation that includes the antigen-binding proteins (e.g., antibodies or antigen-binding fragments).

An “anti-C5” antigen-binding protein specifically binds to C5. The term “specifically binds” refers to those antigen-binding proteins (e.g., mAbs) having a binding affinity to an antigen, such as human C5 protein at 25° C., expressed as K_{D}, of at least about 10⁻⁹ M or less (a lower number) (e.g., about 10⁻¹⁰ M, about 10⁻¹¹ M or about 10⁻¹² M), as measured by real-time, label free bio-layer interferometry assay, for example, at 25° C. or 37° C., e.g., an Octet® HTX biosensor, or by surface plasmon resonance, e.g., BIACORE™, or by solution-affinity ELISA. In some embodiments of the invention, an anti-C5 antigen-binding protein also binds to a variant of C5, e.g., comprising a mutation such as R885H or R885C.

### Pharmaceutical Formulations

The present invention provides pharmaceutical formulations that comprise high concentrations (at least 150 mg/ml or at least 200 mg/ml) of anti-C5 antigen-binding proteins (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4HI2168P; H4HI2169P; H4H12170P; H4H12171P; H4H12175P; H4H12176P2; H4H12177P2; H4H12183P2; H2M11682N; H2M11684N; H2M11694N; H2M11695N; ravulizumab, eculizumab, crovalimab, tesidolumab or mubodina) having a low viscosity (e.g., less than about 15 cP, e.g., about 14 or 14.3) optionally in association with a C5 si-RNA such as cemdisiran. For example, the invention includes a pharmaceutical formulation comprising, consisting of or consisting essentially of: 200 mg/ml pozelimab; 20 mM histidine buffer; 100 mM L-arginine hydrochloride; 2% (w/v) sucrose; 0.15% (w/v) polysorbate-80; and water, pH 5.8.

A pharmaceutical formulation or pharmaceutical composition, as used herein, refers to a formulation/composition including an anti-C5 antigen-binding protein and a pharmaceutically acceptable carrier. A pharmaceutically acceptable carrier includes one or more excipients. In an embodiment of the invention, a pharmaceutical formulation of the present invention is aqueous, i.e., includes water.

Pharmaceutical formulations including anti-C5 antigen-binding proteins may be prepared by admixing the antigen-binding protein with one or more excipients (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

In an embodiment of the invention, a pharmaceutical formulation of the present invention comprises:
≥150 mg/ml, ≥200 mg/ml, ≥250 mg/ml, ≥274 mg/ml or ≥275 mg/ml of anti-C5 antigen-binding protein (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4HI2168P; H4HI2169P; H4H12170P; H4H12171P; H4H12175P; H4H12176P2; H4H12177P2; H4H12183P2; H2M11682N; H2M11684N; H2M11694N; H2M11695N; ravulizumab, eculizumab, crovalimab, tesidolumab or mubodina)
Buffer (e.g., about 20 mM);
An amino acid (e.g., about 100 mM);
An optional sugar (e.g., about 2%);
An optional non-ionic detergent (e.g., about 0.15%);
   and
Water;
   pH about 5-6 (e.g., about pH 5.8).

In an embodiment of the invention, the pharmaceutical formulation of the present invention is aqueous (e.g., suitable for intravenous and/or subcutaneous administration) and comprises an anti-C5 antibody or antigen-binding fragment thereof (e.g., pozelimab) (e.g., about 200 mg/mL or about 180-210 mg/ml), histidine (e.g., histidine-HCl; e.g., about 20 mM or 20 mM±4 mM), pH about 5.8 or 5.8±0.3, arginine (e.g., about 100 mM or 100 mM±20 mM; e.g., L-arginine, L-arginine HCl or L-arginine monohydrochloride), a polyol such as sucrose (e.g., about 2% or 2%±0.4% (w/v)), and a non-ionic surfactant such as polysorbate (e.g., polysorbate 80; e.g., about 0.15% or 0.15%±0.075% (w/v))—e.g.,

200 mg/ml pozelimab;
20 mM histidine buffer;
100 mM L-arginine hydrochloride;
2% (w/v) sucrose;
0.15% (w/v) polysorbate-80;
and water,
pH 5.8.

“Arginine” or “L-arginine” includes any pharmaceutically acceptable salt form thereof, e.g., L-arginine hydrochloride.

Buffers control the pH of formulations and in some cases contribute to the overall stability of a protein product. In an embodiment of the invention, the buffer is a phosphate buffer, acetate buffer, citrate buffer, histidine buffer or imidazole buffer.

An amino acid can be any one of the 20 essential amino acids. In an embodiment of the invention, the amino acid is glycine, arginine, aspartic acid, glutamic acid, lysine, asparagine, glutamine, proline or histidine.

In an embodiment of the invention, the oligosaccharide is sucrose, mannitol, dextrose, glycerol, TMAO (trimethylamine N-oxide), trehalose, ethylene glycol, glycine betaine, xylitol or sorbitol.

Non-ionic detergents contain molecules with head groups that are uncharged. In an embodiment of the invention, the non-ionic detergent is polyoxyethylene-based or glycosidic compound-based. In an embodiment of the invention, the non-ionic detergent is polysorbate-20 (PS20), polysorbate-80 (PS80) or tween-20.

In an embodiment of the invention,
the pharmaceutical formulation of the present invention (e.g., including H4H12166P) has a viscosity of about 8, 9, 10, 11, 12, 13, 14, 14.3, 15, 16, 17, 18, 19 or 20 or 8-20 cP at about 20° C. (e.g., at a concentration of about 200 mg/ml antibody);
the pharmaceutical formulation comprises a viscosity as set forth in Table 8-1 herein (e.g., ±10%) when measured at the indicated temperature, e.g., wherein the antibody is at a concentration as indicated in the Table (e.g., ±1 or 3 or 5 or 10%), e.g., wherein the antibody is H4H12166P, e.g., wherein the antibody is formulated in about 20 mM histidine, pH about 5.8, about 100 mM arginine, about 2% sucrose and about 0.15% polysorbate (e.g., polysorbate 80);
the pharmaceutical formulation of the present invention (e.g., including H4H12166P) has a viscosity of about 50 cP at 20° C. (e.g., at a concentration of about 274 mg/ml antibody);
the pharmaceutical formulation of the present invention (e.g., including H4H12166P) has an osmolality of about 267-404 mmol/kg;
the pharmaceutical formulation of the present invention (e.g., including H4H12166P) contains a % high molecular weight species of about 1.1-2.1 (as measured by SE-UPLC) or 0.1 (as measured by MCE-SDS), at t=0 (i.e., prior to any significant period of storage or incubation);
the pharmaceutical formulation of the present invention (e.g., including H4H12166P) contains a % low molecular weight species of about 0.4-0.5 (as measured by SE-UPLC) or 3.2 (as measured by non-reduced microchip capillary electrophoresis (MCE)-SDS (sodium dodecyl sulfate)), at t=0;
the pharmaceutical formulation of the present invention (e.g., including H4H12166P) contains a % main species of about 98.5 (as measured by SE-UPLC) or 96.7 (as measured by MCE-SDS), at t=0;
the pharmaceutical formulation of the present invention (e.g., including H4H12166P) exhibits an increase of about 0.1 or about 0.2% or less (e.g., about 0%) in high molecular weight (HMW) species after agitation for about 6, 12, 18, 24, 36 or 48 hours, e.g., at 250 rpm; e.g., as measured by SE-UPLC or MCE-SDS;
the pharmaceutical formulation of the present invention (e.g., at an antibody concentration of about 274 mg/ml) (e.g., including H4H12166P) exhibits an increase of about 0% (after up to about 7 days of agitation), about 0.2% (after about 6 months of agitation) or about 0.3% (after about 15.5 months) in high molecular weight (HMW) species, e.g., at 250 rpm; e.g., as measured by SE-UPLC;
the pharmaceutical formulation of the present invention (e.g., including H4H12166P) exhibits an increase of about 0.0, 0.1, 0.2, 0.3, 0.4, 0.5, about 0.6 or about 0.7% in high molecular weight (HMW) species after agitation for about 24 hours or 48 hours, e.g., at 250 rpm;
the pharmaceutical formulation of the present invention (e.g., including H4H12166P) exhibits an increase of about 0% (or less than 0.1%) in high molecular weight (HMW) species after 2 freeze (at −30° C.)—thaw (at room temperature) cycles, e.g., of a 1.5 ml volume in a 5 ml container; e.g., as measured by SE-UPLC;
the pharmaceutical formulation of the present invention (e.g., including H4H12166P) exhibits an increase of about 0 or 0.1% in high molecular weight (HMW) species or low molecular weight (LMW) species after 4 freeze (at −30° C.)—thaw (at room temperature) cycles, e.g., of a 1.5 ml volume in a 5 ml container; e.g., as measured by SE-UPLC;
the pharmaceutical formulation of the present invention (e.g., including H4H12166P) exhibits an increase of about 0.1% in high molecular weight (HMW) species after 8 freeze (at −30° C.)—thaw (at room temperature) cycles, e.g., of a 1.5 ml volume in a 5 ml container; e.g., as measured by SE-UPLC;
the pharmaceutical formulation of the present invention (e.g., including H4H12166P) exhibits an increase of about 0% (or less than about 0.1%) in low molecular weight (LMW) species after 2, 4 or 8 freeze (at −30° C.)—thaw (at room temperature) cycles, e.g., of a 1.5 ml volume in a 5 ml container; e.g., as measured by SE-UPLC;
the anti-C5 antigen-binding protein (e.g., antibody or antigen-binding fragment thereof (e.g., H4H12166P)) in the pharmaceutical formulation of the present invention has Tₘ1 (onset) of about 58.0° C.; Tₘ1 is about 61.7° C.; and a Tₘ2 of about 73.2° C. (e.g., as measured using differential scanning calorimetry (DSC));
one or more methionines in the anti-C5 antigen-binding protein (e.g., antibody or antigen-binding fragment thereof (e.g., H4H12166P)) in the pharmaceutical formulation of the present invention are oxidized (e.g., heavy chain Met105, Met252, Met428 and/or light chain Met4, e.g., of H4H12166P), e.g., at a level of about 6% or less or about 5% or less, about 4% or less, about 3% or less, about 2% or less, about 1% or less, for example, wherein the oxidized methionine is methionine sulfoxide or methionine sulfone, when incubated at 37° C. in the presence of 0 or 1 parts-per-million (ppm) H₂O₂ for about 24 hours;
in a pharmaceutical formulation of the present invention, the heavy chain CDR methionine 105 (e.g., of H4H12166P) is oxidized (e.g., to methionine sulfoxide or methionine sulfone) at about 4.2 or 4.3% when incubated at 37° C. in the presence of 0 or 1 ppm H₂O₂ for about 24 hours,
in a pharmaceutical formulation of the present invention, the level of oxidized heavy chain Met105, Met252, Met428 and/or light chain Met4, e.g., of H4H12166P, when incubated for 24 hours at 37° C. in 1 ppm H₂O₂ does not increase by more than about 0.1 or 0.2% relative to that incubated without H₂O₂ after 24 hours incubation at 37° C. or with no incubation;
the pharmaceutical formulation of the present invention (e.g., including H4H12166P) comprises less than or equal to about 0.1 EU/mg endotoxin content;
the pharmaceutical formulation of the present invention (e.g., including H4H12166P) comprises about 0.9% (or less) high molecular weight (HMW) species (e.g., as measured by size-exclusion ultra-high performance liquid chromatography (SE-UPLC)), e.g., before storage or incubation for a significant period of time;
the pharmaceutical formulation of the present invention (e.g., including H4H12166P) comprises about 0.2% (or less) low molecular weight (LMW) species (e.g., as measured by size-exclusion ultra-high performance liquid chromatography (SE-UPLC)), e.g., before storage or incubation for a significant period of time;
the pharmaceutical formulation of the present invention (e.g., including H4H12166P) comprises about 98.9, 99 or 100% main species (e.g., as measured by size-exclusion ultra-high performance liquid chromatography (SE-UPLC)), e.g., before storage or incubation for a significant period of time;
the pharmaceutical formulation of the present invention (e.g., including H4H12166P) comprises about 0.9, less than about 1.0, about 1.0 or about 1.1 or about 1.2 or about 0.9-1.2% high molecular weight (HMW) species (e.g., as measured by size-exclusion ultra-high performance liquid chromatography (SE-UPLC)) after about 1, about 3 or about 6 months or about 9 months or about 12 months of storage at about 5° C., for example, wherein the percentage of HMW species does not increase by more than about 0.1% or 0.2% or 0.3% after about 1, about 3 or about 6 or about 9 or about 12 months of storage at about 5° C.;
the pharmaceutical formulation of the present invention (e.g., including H4H12166P) comprises about 2, 2.1, 2.2, 2.3 or 2.4% high molecular weight (HMW) species (e.g., as measured by size-exclusion ultra-high performance liquid chromatography (SE-UPLC)) after about 2 days, 7 days, 6 months or 15.5 months of storage at about 5° C., for example, wherein the percentage of HMW species does not increase by more than about 0.1% or 0.2% or 0.3% after about 6 months or 15.5 months of storage at about 5° C.;
the pharmaceutical formulation of the present invention (e.g., including H4H12166P) comprises about 1.1, about 1.2, about 1.3, about 1.4 or about 1.5% or about 1.1-1.5% high molecular weight (HMW) species (e.g., as measured by size-exclusion ultra-high performance liquid chromatography (SE-UPLC)) after about 0.5, about 1, about 3 or about 6 months of storage at about 25° C., e.g., with about 60% relative humidity, e.g., wherein the % HMW species does not increase more than about 0.6% after about 6 month incubation;
the pharmaceutical formulation of the present invention (e.g., including H4H12166P) comprises about 1.3, about 1.4, about 1.9, about 3.8 or about 5.8% or about 1.3-5.8% high molecular weight (HMW) species (e.g., as measured by size-exclusion ultra-high performance liquid chromatography (SE-UPLC)) after about 0.25, about 0.5, about 1, about 2 or about 3 months of storage at about 40° C., e.g., with about 75% relative humidity e.g., wherein the % HMW species does not increase more than about 4.5% after about 3 month incubation;
the pharmaceutical formulation of the present invention (e.g., including H4H12166P) comprises about 0.2, about 0.3, about 0.4, about 0.5 or about 0.4%-0.6 or 0.2-0.4% low molecular weight (LMW) species (e.g., as measured by size-exclusion ultra-high performance liquid chromatography (SE-UPLC)) after about 1, about 3, about 6, about 9 or about 12 months of storage at about 5° C., for example, wherein the percentage of LMW species does not increase by more than about 0.1% or 0.2% or 0.3% or 0.4% after about 1, 3, 6, 9 or 12 months of storage at about 5° C.;
the pharmaceutical formulation of the present invention (e.g., including H4H12166P) comprises about 0.2, about 0.3 or about 0.4 or about 0.2-0.4% low molecular weight (LMW) species (e.g., as measured by size-exclusion ultra-high performance liquid chromatography (SE-UPLC)) after about 0.5, about 1, about 3 or about 6 months of storage at about 25° C., e.g., with about 60% relative humidity, for example, wherein the percentage of LMW species does not increase by more than about 0.1% after about 0.5, 1, 3 or 6 months of storage at about 5° C.;
the pharmaceutical formulation of the present invention (e.g., including H4H12166P) comprises about 0.3, about 0.4, about 0.5, about 0.6, about 0.7, about 0.8 or about 0.3-0.8% low molecular weight (LMW) species (e.g., as measured by size-exclusion ultra-high performance liquid chromatography (SE-UPLC)) after about 0.25, about 0.5, about 1, about 2 or about 3 months of storage at about 40° C., e.g., with about 75% relative humidity, for example, wherein the percentage of LMW species does not increase by more than about 0.1, 0.2, 0.3, 0.4 or 0.5% after about 0.25, 0.5, 1, 2 or 3 months of storage at about 5° C.;
the pharmaceutical formulation of the present invention (e.g., including H4H12166P) comprises about 98, about 98.3, about 98.7, about 98.8, about 99 or about 98-99% main species (e.g., as measured by size-exclusion ultra-high performance liquid chromatography (SE-UPLC)) after about 1, about 3, about 6, about 9 months or about 12 months of storage at about 5° C., for example, wherein the percentage of main species does not decrease by more than about 0.1, 0.2, 0.3, 0.4, 0.5 or 0.6% after about 1, 3, 6, 9 or 12 months of storage at about 5° C.;
the pharmaceutical formulation of the present invention (e.g., including H4H12166P) comprises about 98, about 98.6, about 98.4, about 98.7, about 98.8, about 98.1, about 99 or about 98-99% main species (e.g., as measured by size-exclusion ultra-high performance liquid chromatography (SE-UPLC)) after about 0.5, about 1, about 3 or about 6 months of storage at about 25° C. e.g., with about 60% relative humidity, for example, wherein the percentage of main species does not decrease by more than about 0.1, 0.2, 0.3, 0.4, 0.5, 0.6, 0.7 or 0.8% after about 1, 3 or 6 months of storage at about 25° C.;
the pharmaceutical formulation of the present invention (e.g., including H4H12166P) comprises about 98.3, about 98.4, about 97.6, about 95.5, about 93.4, or about 93.4-98.4, or about 93-98% main species (e.g., as measured by size-exclusion ultra-high performance liquid chromatography (SE-UPLC)) after about 0.25, about 0.5, about 1, about 2 or about 6 months of storage at about 40° C. e.g., with about 75% relative humidity for example, wherein the percentage of main species does not decrease by more than about 1, 2, 3, 4, 5 or 5.5% after about 0.25, 0.5, 1, 2 or 3 months of storage at about 40° C.;
the pharmaceutical formulation of the present invention (e.g., including H4H12166P) comprises about 29% acidic charge variants, about 11% basic charge variants and/or about 60% main species, e.g., as measured by imaging capillary isoelectric focusing (iCIEF), e.g., before storage or incubation for a significant period of time;
the pharmaceutical formulation of the present invention (e.g., including H4H12166P) comprises about 30, 31 or 32% acidic charge variants, about 14, 13, 12 or 11% basic charge variants and/or about 56 or 57% main species, e.g., as measured by imaging capillary isoelectric focusing (iCIEF), e.g., after about 6, 9 or 12 months of storage at about 5° C.; and/or does not exhibit an increase of acidic charge variants of more than about 3.0 or 3.1% after 12 months storage at 5° C., and/or does not exhibit a decrease in main species of more than about 3.0 or 3.1% after 12 months storage at 5° C., and/or does not exhibit an increase of more than about 0.1 or 0% basic charge variants after 12 months storage at 5° C.;
the pharmaceutical formulation of the present invention (e.g., including H4H12166P) comprises about 33% acidic charge variants, about 20% basic charge variants and/or about 47% main species, e.g., as measured by imaging capillary isoelectric focusing (iCIEF), e.g., after about 6 months of storage at about 25° C.;
the pharmaceutical formulation of the present invention (e.g., including H4H12166P) comprises about 45% acidic charge variants, about 36% basic charge variants and/or about 20% main species, e.g., as measured by imaging capillary isoelectric focusing (iCIEF), e.g., after about 3 months of storage at about 40° C.;
when a pharmaceutical formulation of the present invention (e.g., including H4H12166P) is stored at about 37° C., it forms HMW species at a rate of about 0.6% per month and/or forms acidic variants at rate of about 0.6% per month;
when a pharmaceutical formulation of the present invention (e.g., including H4H12166P) is stored at about 40° C. it forms HMW species at a rate of about 1.2% per month and/or forms acidic variants at rate of about 3.1% per month; and/or
when a pharmaceutical formulation of the present invention (e.g., including H4H12166P) is stored at about 45° C. it forms HMW species at a rate of about 2.6% per month and/or forms acidic variants at rate of about 8.8% per month;
   for example, wherein the formulation includes buffer, L-arginine, water, optionally an oligosaccharide, optionally a non-ionic detergent, and has a pH of about 5.8 (e.g., with a viscosity of about 14.3 or about 14 or about 15 at 20° C.).

The present invention includes embodiments wherein any one or more selected from the foregoing characterize any of the anti-C5 antigen-binding proteins which are described herein.

In an embodiment of the invention, the formulation comprises:
About 150 mg/ml, 175 mg/ml, 200 mg/ml, 211 mg/ml, 220 mg/ml, 242 mg/ml or 274 mg/ml anti-C5 antibody or antigen-binding fragment thereof (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; H2M11695N; ravulizumab, eculizumab, crovalimab, tesidolumab or mubodina)
Histidine buffer (e.g., about 20 mM),
L-arginine (e.g., L-arginine HCl) (e.g., about 100 mM)
Optionally, sucrose (e.g., about 2% (w/v))
Optionally, polysorbate-80 (PS-80) (e.g., about 0.15% (w/v));
   and
Water;
   pH about 5.8;
   e.g., having a viscosity of about 13.2-16.7 or 14 or about 14.3 or about 15 cP (e.g., at 20° C.).

The present invention includes a pharmaceutical formulation comprising about 200 mg/ml H2M11683N; about 20 mM histidine buffer; about 100 mM L-arginine; about 2% (w/v) sucrose; about 0.15% (w/v) polysorbate-80 (PS-80) and water, pH 5.8±0.2.

The present invention includes a pharmaceutical formulation comprising about 200 mg/ml H2M11686N; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H4H12159P; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H4H12161P; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H4H12163P; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H4H12164P; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H4H12166P; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H4H12166P2; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H4H12166P3; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H4H12166P4; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H4H12166P5; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H4H12166P6; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H4H12166P7; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H4H12166P8; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H4H12166P9; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H4H12167P; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H4H12168P; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H4H12169P; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H4H12170P; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H4H12171P; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H4H12175P; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H4H12176P2; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H4H12177P2; about 20 mM histidine buffer; about 100 mM L-arginine; about 2% (w/v) (w/v) sucrose; about 0.15% (w/v) (w/v) polysorbate-80 and water, pH 5.8±0.2.

The present invention includes a pharmaceutical formulation comprising about 200 mg/ml H4H12183P2; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H2M11682N; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H2M11684N; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H2M11694N; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H2M11695N; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml ravulizumab; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml crovalimab; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml eculizumab; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml tesidolumab; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml mubodina; about 20 mM histidine buffer; about 100 mM L-arginine; about 2% (w/v) sucrose; about 0.15% (w/v) polysorbate-80 and water, pH 5.8±0.2.

The present invention includes a pharmaceutical formulation comprising about 200 mg/ml H4H12166P, about 5 mM histidine, about 2.5% (w/v) proline, about 5% (w/v) sucrose, about 150 mM L-arginine-HCl, about 0.2% (w/v) PS-80, and water, pH about 5.7.

The present invention includes a pharmaceutical formulation comprising about 135 mg/ml H4H12166P, about 20 mM histidine, about 5% (w/v) proline, about 10% (w/v) sucrose, about 150 mM L-arginine-HCl, about 0.02% (w/v) PS-80, and water, pH about 5.7.

The present invention includes a pharmaceutical formulation comprising about 160 mg/ml H4H12166P, about 5 mM histidine, about 5% (w/v) sucrose, about 75 mM L-arginine-HCl, about 0.02% (w/v) PS-80, and water, pH about 6.2.

The present invention includes a pharmaceutical formulation comprising about 120 mg/ml H4H12166P, about 40 mM histidine, about 5% (w/v) proline, about 0.02% (w/v) PS-80, and water, pH about 6.8.

The present invention includes a pharmaceutical formulation comprising about 120 mg/ml H4H12166P, about 40 mM histidine, about 0.02% (w/v) PS-80, and water, pH about 5.7.

The present invention includes a pharmaceutical formulation comprising about 160 mg/ml H4H12166P, about 5 mM histidine, about 2.5% (w/v) proline, about 10% (w/v) sucrose, about 0.2% (w/v) PS-80, and water, pH about 6.8.

The present invention includes a pharmaceutical formulation comprising about 120 mg/ml H4H12166P, about 40 mM histidine, about 5% (w/v) proline, about 150 mM L-arginine-HCl, about 0.02% (w/v) PS-80, and water, pH about 5.7.

The present invention includes a pharmaceutical formulation comprising about 200 mg/ml H4H12166P, about 5 mM histidine, about 0.2% (w/v) PS-80, and water, pH about 5.7.

The present invention includes a pharmaceutical formulation comprising about 200 mg/ml H4H12166P, about 20 mM histidine, about 5% (w/v) proline, about 10% (w/v) sucrose, about 0.2% (w/v) PS-80, and water, pH about 6.2.

The present invention includes a pharmaceutical formulation comprising about 120 mg/ml H4H12166P, about 40 mM histidine, about 150 mM L-arginine-HCl, about 0.02% (w/v) PS-80, and water, pH about 6.8.

The present invention includes a pharmaceutical formulation comprising about 200 mg/ml H4H12166P, about 5 mM histidine, about 5% (w/v) proline, about 150 mM L-arginine-HCl, about 0.2% (w/v) PS-80, and water, pH about 6.2.

The present invention includes a pharmaceutical formulation comprising about 120 mg/ml H4H12166P, about 20 mM histidine, about 5% (w/v) proline, about 5% (w/v) sucrose, about 150 mM L-arginine-HCl, about 0.2% (w/v) PS-80, and water, pH about 6.8.

The present invention includes a pharmaceutical formulation comprising about 120 mg/ml H4H12166P, about 5 mM histidine, about 10% (w/v) sucrose, about 0.2% (w/v) PS-80, and water, pH about 5.7.

The present invention includes a pharmaceutical formulation comprising about 120 mg/ml H4H12166P, about 5 mM histidine, about 0.2% (w/v) PS-80, and water, pH about 6.8.

The present invention includes a pharmaceutical formulation comprising about 120 mg/ml H4H12166P, about 5 mM histidine, about 150 mM L-arginine-HCl, about 0.2% (w/v) PS-80, and water, pH about 5.7.

The present invention includes a pharmaceutical formulation comprising about 175 mg/ml H4H12166P, about 40 mM histidine, about 5% (w/v) proline, about 0.2% (w/v) PS-80, and water, pH about 5.7.

The present invention includes a pharmaceutical formulation comprising about 200 mg/ml H4H12166P, about 5 mM histidine, about 10% (w/v) sucrose, about 150 mM L-arginine-HCl, about 0.2% (w/v) PS-80, and water, pH about 6.8.

The present invention includes a pharmaceutical formulation comprising about 185 mg/ml H4H12166P, about 40 mM histidine, about 10% (w/v) sucrose, about 0.02% (w/v) PS-80, and water, pH about 5.7.

The present invention includes a pharmaceutical formulation comprising about 120 mg/ml H4H12166P, about 40 mM histidine, about 10% (w/v) sucrose, about 150 mM L-arginine-HCl, about 0.2% (w/v) PS-80, and water, pH about 5.7.

The present invention includes a pharmaceutical formulation comprising about 120 mg/ml H4H12166P, about 40 mM histidine, about 10% (w/v) sucrose, about 0.02% (w/v) PS-80, and water, pH about 6.8.

The present invention includes a pharmaceutical formulation comprising about 120 mg/ml H4H12166P, about 5 mM histidine, about 5% (w/v) proline, about 0.02% (w/v) PS-80, and water, pH about 5.7.

The present invention includes a pharmaceutical formulation comprising about 170 mg/ml H4H12166P, about 40 mM histidine, about 5% (w/v) proline, about 10% (w/v) sucrose, about 150 mM L-arginine-HCl, about 0.02% (w/v) PS-80, and water, pH about 6.8.

The present invention includes a pharmaceutical formulation comprising about 120 mg/ml H4H12166P, about 40 mM histidine, about 5% (w/v) proline, about 10% (w/v) sucrose, about 0.02% (w/v) PS-80, and water, pH about 5.7.

The present invention includes a pharmaceutical formulation comprising about 120 mg/ml H4H12166P, about 5 mM histidine, about 2.5% (w/v) proline, about 10% (w/v) sucrose, about 150 mM L-arginine-HCl, about 0.02% (w/v) PS-80, and water, pH about 6.2.

The present invention includes a pharmaceutical formulation comprising about 200 mg/ml H4H12166P, about 5 mM histidine, about 5% (w/v) proline, about 10% (w/v) sucrose, about 75 mM L-arginine-HCl, about 0.2% (w/v) PS-80, and water, pH about 5.7.

The present invention includes a pharmaceutical formulation comprising about 120 mg/ml H4H12166P, about 5 mM histidine, about 5% (w/v) proline, about 10% (w/v) sucrose, about 75 mM L-arginine-HCl, about 0.2% (w/v) PS-80, and water, pH about 6.8.

The present invention includes a pharmaceutical formulation comprising about 160 mg/ml H4H12166P, about 5 mM histidine, about 5% (w/v) proline, about 150 mM L-arginine-HCl, about 0.2% (w/v) PS-80, and water, pH about 6.8.

The present invention includes a pharmaceutical formulation comprising about 200 mg/ml H4H12166P, about 20 mM histidine, about 2.5% (w/v) proline, about 75 mM L-arginine-HCl, about 0.02% (w/v) PS-80, and water, pH about 6.8.

The present invention includes a pharmaceutical formulation comprising about 170 mg/ml H4H12166P, about 35 mM histidine, about 150 mM L-arginine-HCl, about 0.02% (w/v) PS-80, and water, pH about 5.7.

The present invention includes a pharmaceutical formulation comprising about 183 mg/ml H4H12166P, about 40 mM histidine, about 0.2% (w/v) PS-80, and water, pH about 6.8.

The present invention includes a pharmaceutical formulation comprising about 200 mg/ml H4H12166P, about 5 mM histidine, about 5% (w/v) proline, about 5% (w/v) sucrose, about 0.02% (w/v) PS-80, and water, pH about 6.8.

The present invention includes a pharmaceutical formulation comprising about 160 mg/ml H4H12166P, about 40 mM histidine, about 2.5% (w/v) proline, about 5% (w/v) sucrose, about 75 mM L-arginine-HCl, about 0.2% (w/v) PS-80, and water, pH about 6.2.

The present invention includes a pharmaceutical formulation comprising about 187 mg/ml H4H12166P, about 40 mM histidine, about 0.02% (w/v) PS-80, and water, pH about 5.7.

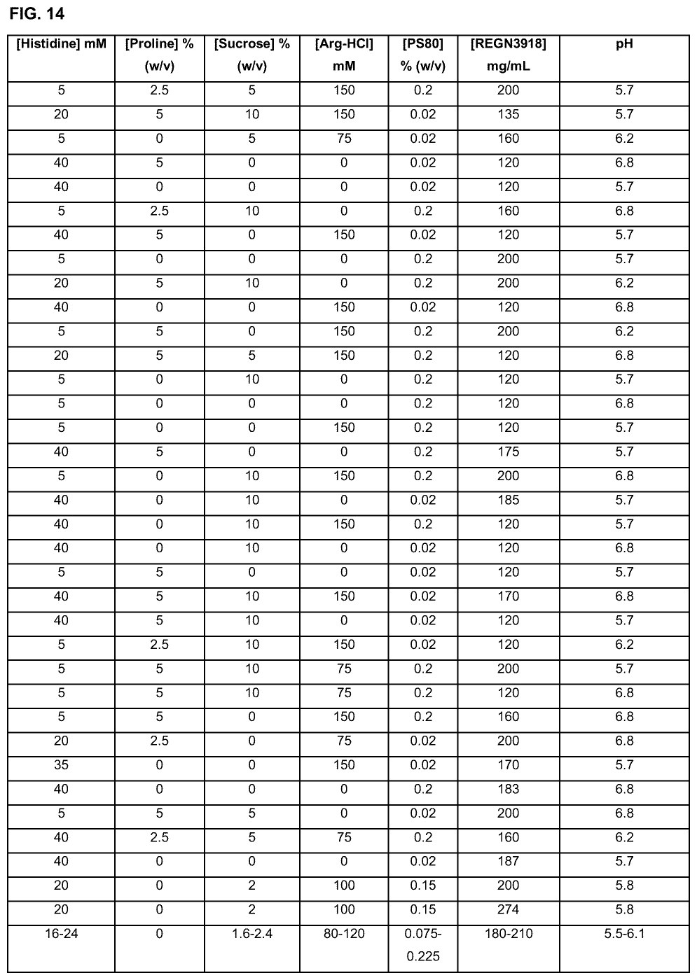

See, for example, FIG. 14. The present invention includes any of the pharmaceutical formulations described in FIG. 14.

The present invention provides a vessel (e.g., a plastic or glass vial, e.g., with a cap, or a chromatography column, hollow bore needle or a syringe cylinder) comprising a pharmaceutical formulation of the present invention that includes an anti-C5 antigen-binding protein, e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4HI2168P; H4HI2169P; H4H12170P; H4H12171P; H4H12175P; H4H12176P2; H4H12177P2; H4H12183P2; H2M11682N; H2M11684N; H2M11694N; H2M11695N; ravulizumab, eculizumab, crovalimab, tesidolumab or mubodina.

The present invention also provides an injection device comprising a pharmaceutical formulation of the present invention including an anti-C5 antigen-binding protein, e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4HI2168P; H4HI2169P; H4H12170P; H4H12171P; H4H12175P; H4H12176P2; H4H12177P2; H4H12183P2; H2M11682N; H2M11684N; H2M11694N; H2M11695N; ravulizumab, eculizumab, crovalimab tesidolumab or mubodina. The injection device may be packaged into a kit. An injection device is a device that introduces a substance into the body of a subject via a parenteral route, e.g., intraocular, intravitreal, intramuscular, subcutaneous or intravenous. For example, an injection device may be a syringe or an auto-injector (e.g., pre-filled with the pharmaceutical formulation) which, for example, includes a cylinder or barrel for holding fluid to be injected (e.g., comprising the antibody or fragment or a pharmaceutical formulation thereof), a needle for piecing skin, blood vessels or other tissue for injection of the fluid; and a plunger for pushing the fluid out of the cylinder and through the needle bore and into the body of the subject.

The present invention also includes a kit comprising a vessel (e.g., a vial) or injection device comprising (a) a pharmaceutical formulation of the present invention including an anti-C5 antigen-binding protein, e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4HI2168P; H4HI2169P; H4H12170P; H4H12171P; H4H12175P; H4H12176P2; H4H12177P2; H4H12183P2; H2M11682N; H2M11684N; H2M11694N; H2M11695N; ravulizumab, eculizumab, crovalimab, tesidolumab or mubodina; and (b) a vessel (e.g., a vial) or injection device comprising an oligonucleotide, for example, cemdisiran, or a pharmaceutical formulation thereof that comprises a pharmaceutically acceptable carrier and, optionally, one or more additional materials such as, for example, written materials (e.g., instructions for use).

To prepare pharmaceutical formulations of the present invention, an anti-C5 antigen-binding protein, e.g., antibody or antigen-binding fragment thereof (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4HI2168P; H4HI2169P; H4H12170P; H4H12171P; H4H12175P; H4H12176P2; H4H12177P2; H4H12183P2; H2M11682N; H2M11684N; H2M11694N; H2M11695N; ravulizumab, eculizumab, crovalimab, tesidolumab or mubodina), is mixed with the required excipients (e.g., histidine, arginine, sucrose, PS-80 and water) and, optionally, a further therapeutic agent. Optionally, the pharmaceutical composition is then lyophilized. Such methods and pharmaceutical formulations, which are the product of such methods, are also part of the present invention.

In an embodiment of the invention, a pharmaceutical formulation of the present invention includes no more than one anti-C5 antigen-binding protein. In an embodiment of the invention, a pharmaceutical formulation of the present invention includes more than one anti-C5 antigen-binding protein, e.g., 2 or 3. In an embodiment of the invention, when two or more anti-C5 antigen-binding proteins are in a pharmaceutical formulation of the present invention, two or more of the antigen-binding proteins do not compete for binding to C5 (e.g., H4H12176P2+H4H12177P2; H4H12166P8+H4H12170P; H4H12166P+H4H12170P; H4H12166P+H4H12161P; H4H12166P+H4H12171P; H4H12166P+H4H12175P; H4H12166P+H4H12176P2 or H4H12166P+H4H12177P2). In an embodiment of the invention, when two or more anti-C5 antigen-binding proteins are present, they do compete for binding to C5.

### Combinations

The present invention provides pharmaceutical formulations that include an anti-C5 antigen-binding protein (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4HI2168P; H4HI2169P; H4H12170P; H4H12171P; H4H12175P; H4H12176P2; H4H12177P2; H4H12183P2; H2M11682N; H2M11684N; H2M11694N; H2M11695N; ravulizumab, eculizumab, crovalimab, tesidolumab or mubodina) in association with one or more further therapeutic agents; as well as methods of use thereof and methods of making such compositions.

In an embodiment of the invention, the further therapeutic agent is an oligonucleotide (e.g., DNA or RNA or a duplex of both), e.g., that binds to DNA or mRNA encoding C5. In an embodiment of the invention, the oligonucleotide is up to about 23, about 19-22, about 19-23 or about 19, about 20, about 21, about 22 or about 23 nucleotides in length (e.g., a 19-23 nucleotide RNA molecule). In an embodiment of the invention, the oligonucleotide is single stranded (e.g., in anti-sense orientation) or double stranded. A double stranded oligonucleotide includes a strand in sense orientation and a strand in an anti-sense orientation. In an embodiment of the invention, the double stranded oligonucleotide (e.g., RNA) has a 3′ overhang and/or a 5′ overhang, for example, of at least two nucleotides. In an embodiment of the invention, the oligonucleotide is naked and in another embodiment the oligonucleotide is chemically modified.

In an embodiment of the invention, the further therapeutic agent is an RNAi agent that binds to an RNA encoding C5 or a portion thereof. An RNAi agent refers to an agent that contains RNA and which mediates the targeted cleavage of an RNA transcript via an RNA-induced silencing complex (RISC) pathway. RNAi directs the sequence-specific degradation of mRNA through a process known as RNA interference. The RNAi modulates, e.g., inhibits, the expression of C5 in a cell, e.g., a cell within a subject, such as a mammalian subject.

In one embodiment of the invention, an RNAi agent of the invention includes a single stranded RNA that interacts with a target RNA sequence, e.g., a C5 target mRNA sequence, to direct the cleavage of the target RNA. Without wishing to be bound by theory it is believed that long double stranded RNA introduced into cells is broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in one aspect the invention relates to a single stranded RNA (siRNA) generated within a cell and which promotes the formation of a RISC complex to effect silencing of the target gene, i.e., a C5 gene. Accordingly, the term “siRNA” is also used herein to refer to an RNAi as described herein.

In another embodiment, the RNAi agent may be a single-stranded siRNA that is introduced into a cell or organism to inhibit a target mRNA. In an embodiment of the invention, single-stranded RNAi agents bind to the RISC endonuclease, Argonaute 2, which then cleaves the target mRNA. The single-stranded siRNAs are, in an embodiment of the invention, 15-30 nucleotides and are chemically modified. The design and testing of single-stranded siRNAs are described in U.S. Pat. No. 8,101,348 and in Lima et al., (2012) Cell 150: 883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150:883-894.

In another embodiment of the invention, an RNAi agent is a double-stranded RNA (dsRNA). A dsRNA, refers to a complex of ribonucleic acid molecules, having a duplex structure comprising two anti-parallel and substantially complementary nucleic acid strands, referred to as having “sense” and “antisense” orientations with respect to a target RNA, i.e., a C5 gene. In some embodiments of the invention, a double-stranded RNA (dsRNA) triggers the degradation of a target RNA, e.g., an mRNA, through a post-transcriptional gene-silencing mechanism referred to as RNA interference.

In an embodiment of the invention, the oligonucleotide (e.g., RNAi) is conjugated to another molecule such as a sugar, such as an N-acetylgalactosamine (GalNAc) derivative such as

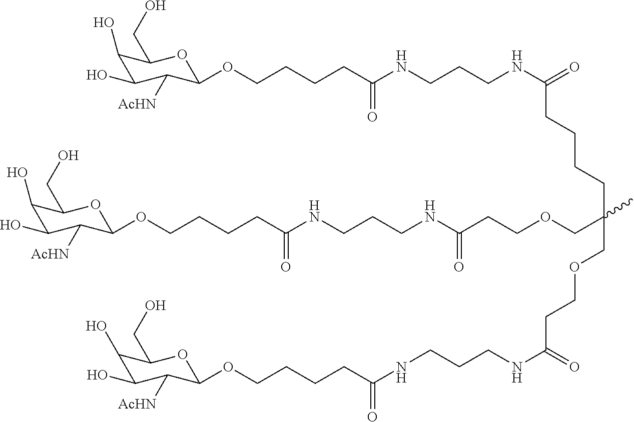 [SMILES: CC(=O)NC1C(OCCCCC(=O)NCCCNC(=O)CCCCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In an embodiment of the invention, the oligonucleotide (e.g., RNAi) is conjugated to another molecule as shown in the following schematic:

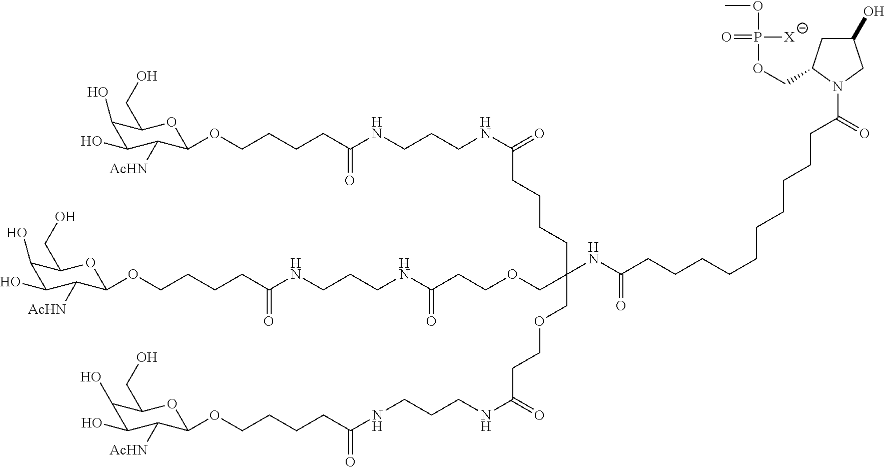

wherein X is O or S.

In an embodiment of the invention, the further therapeutic agent is cemdisiran. In an embodiment of the invention, the further therapeutic agent is a double stranded RNA comprising the anti-sense strand nucleotide sequence:

(SEQ ID NO: 358)
5′-UAUUAUAAAAAUAUCUUGCUUUU-3′;

and/or the sense strand comprises the nucleotide sequence:

(SEQ ID NO: 359)
5′-AAGCAAGAUAUUUUUAUAAUA-3′.

In an embodiment of the invention, the further therapeutic agent is a double-stranded ribonucleic acid (dsRNA) agent for inhibiting expression of complement component C5, wherein said dsRNA agent comprises a sense strand and an antisense strand, wherein the sense strand comprises:

(SEQ ID NO: 360)
5′-asasGfcAfaGfaUfAfUfuUfuuAfuAfaua-3′

and the antisense strand comprises:

(SEQ ID NO: 361)
5′-usAfsUfuAfuaAfaAfauaUfcUfuGfcuususudTdT-3′,

wherein a, g, c and u are 2′-O-methyl (2′-OMe) A, G, C, and U, respectively; Af, Gf, Cf and Uf are 2′-fluoro A, G, C and U, respectively; dT is a deoxy-thymine nucleotide; and s is a phosphorothioate linkage; and wherein said sense strand is conjugated at the 3′-terminus to the ligand:

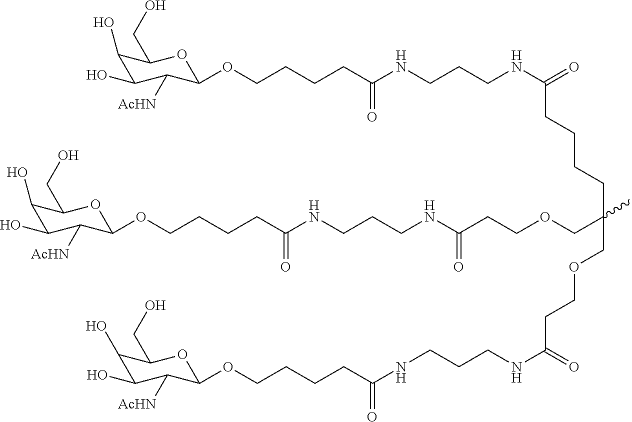 [SMILES: CC(=O)NC1C(OCCCCC(=O)NCCCNC(=O)CCCCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

See U.S. Pat. No. 9,249,415.

In an embodiment of the invention, the RNAi is in a pharmaceutical formulation comprising a lipid nanoparticle (LNP). An LNP is a vesicle comprising a lipid layer encapsulating a pharmaceutically active molecule such as RNAi. LNPs are described in, for example, U.S. Pat. Nos. 6,858,225, 6,815,432, 8,158,601, and 8,058,069, the entire contents of which are hereby incorporated by reference.

In an embodiment of the invention, the further therapeutic agent is an anti-coagulant, warfarin, aspirin, heparin, phenindione, fondaparinux, idraparinux, a thrombin inhibitor, argatroban, lepirudin, bivalirudin, dabigatran, an anti-inflammatory drug, a corticosteroid, a non-steroidal anti-inflammatory drug (NSAID), an antihypertensive, an angiotensin-converting enzyme inhibitor, an immunosuppressive agent, vincristine, cyclosporine A, or methotrexate, a fibrinolytic agent ancrod, E-aminocaproic acid, antiplasmin-a1, prostacyclin, defibrotide, a lipid-lowering agent, an inhibitor of hydroxymethylglutaryl CoA reductase, an anti-CD20 agent, rituximab, an anti-TNFalpha agent, infliximab, an anti-seizure agent, magnesium sulfate, a C3 inhibitor and/or an anti-thrombotic agent.

The term “in association with” indicates that components of a pharmaceutical formulation, (1) an anti-C5 antigen-binding protein and pharmaceutically acceptable carrier components, along with (2) one or more further therapeutic agents, such as cemdisiran, can be formulated into a single composition, e.g., for simultaneous delivery, or formulated separately into two or more compositions (e.g., a kit including each component, for example, wherein the further therapeutic agent is in a separate formulation). Components administered in association with each another can be administered to a subject at the same time or at a different time than when the other component is administered; for example, each administration may be given simultaneously (e.g., together in a single composition or essentially simultaneously during the same administration session) or non-simultaneously at one or more intervals over a given period of time. Moreover, the separate components administered in association with each another may be administered to a subject by the same or by a different route.

### Administration and Treatment

The pharmaceutical formulations of the present invention are useful for the treatment or prevention of a C5-associated disease and/or for ameliorating at least one sign or symptom associated with such C5-associated disease.

The term “C5-associated disease” refers to a disease, disorder, condition or syndrome which is caused, maintained or exacerbated, or whose signs and/or symptoms are caused, maintained or exacerbated, directly or indirectly, by complement system activity wherein the complement system activity can be reduced or stabilized or eliminated by inhibition of C5 activity. Such C5 activity can be inhibited by preventing, for example, cleavage of C5 precursor into C5a and C5b chains and/or formation of membrane attack complex (MAC).

Treatment of a C5-associated disease refers to the reduction, stabilization or elimination of the disease and/or one or more of its signs and/or symptoms thereof.

Subjective evidence of a disease, disorder, condition or syndrome is a symptom. A sign is objective evidence of the disease, disorder, condition or syndrome. For example, blood coming out a nostril is a sign insofar as it is apparent to the patient, physician, and others. Anxiety, low back pain, and fatigue are symptoms insofar as only the patient can perceive them.

The term “subject” includes a mammal such as a human, mouse, goat, rabbit, rat, dog, non-human primate or monkey. In an embodiment of the invention, amino acid Arginine 885 is mutated in the subject's C5 to another amino acid, e.g., R885H or R885C.

The pharmaceutical formulations of the present invention are useful for treating or preventing a C5-associated disease which is one or more of:
adult respiratory distress syndrome
age-related macular degeneration (AMD)
allergy
Alport's syndrome
Alzheimer's disease
antiphospholipid syndrome (APS)
asthma
atherosclerosis
atypical hemolytic uremic syndrome (aHUS)
an autoimmune disease
autoimmune hemolytic anemia (AIHA)
balloon angioplasty
bronchoconstriction
bullous pemphigoid
burns
C3 glomerulopathy
capillary leak syndrome
a cardiovascular disorder
catastrophic antiphospholipid syndrome (CAPS)
a cerebrovascular disorder
CHAPLE disease (CD55 deficiency with hyperactivation of complement, angiopathic thrombosis, and protein-losing enteropathy)
a chemical injury
chronic obstructive pulmonary disease (COPD)
cold agglutinin disease (CAD)
corneal and/or retinal tissue
Crohn's disease
Degos disease
dense deposit disease (DDD)
dermatomyositis
diabetes
diabetic angiopathy
diabetic macular edema (DME)
diabetic nephropathy
diabetic retinopathy
dilated cardiomyopathy
disorder of inappropriate or undesirable complement activation
dyspnea
emphysema
epidermolysis bullosa
epilepsy
fibrogenic dust disease
frostbite
geographic atrophy (GA)
glomerulonephritis
glomerulopathy
Goodpasture's Syndrome
Graves' disease
Guillain-Barre Syndrome
Hashimoto's thyroiditis
hemodialysis complications
hemolysis-elevated liver enzymes-and low platelets (HELLP) syndrome
hemolytic anemia
hemoptysis
Henoch-Schonlein purpura nephritis
hereditary angioedema
hyperacute allograft rejection
hypersensitivity pneumonitis
idiopathic thrombocytopenic purpura (ITP)
IgA nephropathy
an immune complex disorder
immune complex vasculitis
immune complex-associated inflammation
an infectious disease
inflammation caused by an autoimmune disease
an inflammatory disorder
inherited CD59 deficiency
injury due to inert dusts and/or minerals
interleukin-2 induced toxicity during IL-2 therapy
ischemia-reperfusion injury
Kawasaki's disease
a lung disease or disorder
lupus nephritis
membrane proliferative glomerulonephritis
membrano-proliferative nephritis
mesenteric artery reperfusion after aortic reconstruction
mesenteric/enteric vascular disorder
multifocal motor neuropathy (MMN)
multiple sclerosis
myasthenia gravis
myocardial infarction
myocarditis
neurological disorder
neuromyelitis optica
obesity
ocular angiogenesis
ocular neovascularization affecting choroidal
organic dust disease
parasitic disease
Parkinson's disease
paroxysmal nocturnal hemoglobinuria (PNH)
pauci-immune vasculitis
pemphigus
percutaneous transluminal coronary angioplasty (PTCA)
peripheral (e.g., musculoskeletal) vascular disorder
pneumonia
post-ischemic reperfusion condition
post-pump syndrome in cardiopulmonary bypass
post-pump syndrome in renal bypass
progressive kidney failure
proliferative nephritis
proteinuric kidney disease
psoriasis
pulmonary embolism
pulmonary fibrosis
pulmonary infarction
pulmonary vasculitis
recurrent fetal loss
a renal disorder
renal ischemia
renal ischemia-reperfusion injury
a renovascular disorder
restenosis following stent placement
rheumatoid arthritis (RA)
rotational atherectomy
schizophrenia
sepsis
septic shock
SLE nephritis
smoke injury
spinal cord injury
spontaneous fetal loss
stroke
systemic inflammatory response to sepsis
systemic lupus erythematosus (SLE)
systemic lupus erythematosus-associated vasculitis
Takayasu's disease
thermal injury
thrombotic thrombocytopenic purpura (TTP)
traumatic brain injury
type I diabetes
typical hemolytic uremic syndrome (tHUS)
uveitis
vasculitis
vasculitis associated with rheumatoid arthritis
venous gas embolus (VGE); and/or
xenograft rejection

Thus, the present invention includes methods for treating or preventing a C5-associated disease (e.g., PNH, aHUS or CHAPLE), in a subject in need thereof e.g., in a subject suffering from the C5-associated disease, comprising administering a therapeutically effective amount of pharmaceutical formulation of the present invention that includes an anti-C5 antigen-binding protein (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4HI2168P; H4HI2169P; H4H12170P; H4H12171P; H4H12175P; H4H12176P2; H4H12177P2; H4H12183P2; H2M11682N; H2M11684N; H2M11694N; H2M11695N; ravulizumab, crovalimab or eculizumab) to the subject, optionally in association with a further therapeutic agent (e.g., cemdisiran). In an embodiment of the invention, the subject had previously received a different anti-C5 antigen-binding protein, e.g., ravulizumab, crovalimab or eculizumab.

Paroxysmal nocturnal hemoglobinuria (PNH) is a rare acquired, life-threatening disease of the blood. The disease is characterized by destruction of red blood cells (hemolytic anemia), blood clots (thrombosis), and impaired bone marrow function (not making enough of the three blood components). Signs and symptoms of PNH can include significant fatigue or weakness, bruising or bleeding easily, shortness of breath, recurring infections and/or flu-like symptoms, difficulty in controlling bleeding, even from very minor wounds, the appearance of small red dots on the skin that indicates bleeding under the skin, severe headache, fever due to infection and blood clots (thrombosis). Thus, the present invention provides a method for treating or preventing PNH in a subject in need thereof comprising administering a therapeutically effective amount of anti-C5 antigen-binding protein (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4HI2169P; H4H12170P; H4H12171P; H4H12175P; H4H12176P2; H4H12177P2; H4H12183P2; H2M11682N; H2M11684N; H2M11694N; H2M11695N; ravulizumab, crovalimab or eculizumab) in a pharmaceutical formulation of the present invention to the subject. The present invention includes a method for reducing, stabilizing and/or eliminating one or more signs and/or symptoms of PNH (e.g., hemolytic anemia) in a subject suffering from PNH and said signs and/or symptoms comprising administering a therapeutically effective amount of pharmaceutical formulation of the present invention comprising an anti-C5 antigen-binding protein to the subject.

Atypical hemolytic uremic syndrome (aHUS) is a rare disease characterized by low levels of circulating red blood cells due to their destruction (hemolytic anemia), low platelet count (thrombocytopenia) due to their consumption and inability of the kidneys to process waste products from the blood and excrete them into the urine (acute kidney failure), a condition known as uremia. Signs and symptoms of aHUS can include, for example, feelings of illness, fatigue, irritability, and lethargy, anemia, thrombocytopenia, acute kidney failure, hypertension and organ damage. Thus, the present invention provides a method for treating or preventing aHUS in a subject in need thereof comprising administering a therapeutically effective amount of anti-C5 antigen-binding protein (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4HI2169P; H4H12170P; H4H12171P; H4H12175P; H4H12176P2; H4H12177P2; H4H12183P2; H2M11682N; H2M11684N; H2M11694N; H2M11695N; ravulizumab, crovalimab or eculizumab) in a pharmaceutical formulation of the present invention to the subject. The present invention includes a method for reducing, stabilizing and/or eliminating one or more signs and/or symptoms of aHUS (e.g., hemolytic anemia) in a subject suffering from aHUS and said signs and/or symptoms comprising administering a therapeutically effective amount of pharmaceutical formulation of the present invention comprising an anti-C5 antigen-binding protein to the subject.

CHAPLE disease is an autosomal recessive disorder caused by loss of function mutations in CD55 (also known as decay accelerating factor, DAF). Signs and symptoms of CHAPLE can include hypoproteinemia (low serum levels of albumin and immunoglobulins)-hypoproteinemia leads to facial and extremity edema and recurrent infections, malabsorption syndrome (chronic diarrhea, failure to thrive, anemia, and micronutrient deficiencies), complement overactivation, intestinal lymphangiectasia (IL) and bowel inflammation; and/or increased susceptibility to visceral thrombosis. Thus, the present invention provides a method for treating or preventing CHAPLE disease in a subject in need thereof comprising administering a therapeutically effective amount of anti-C5 antigen-binding protein (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4HI2169P; H4H12170P; H4H12171P; H4H12175P; H4H12176P2; H4H12177P2; H4H12183P2; H2M11682N; H2M11684N; H2M11694N; H2M11695N; ravulizumab, crovalimab or eculizumab) in a pharmaceutical formulation of the present invention to the subject. The present invention includes a method for reducing, stabilizing and/or eliminating one or more signs and/or symptoms of CHAPLE (e.g., hypoproteinemia) in a subject suffering from CHAPLE and said signs and/or symptoms comprising administering a therapeutically effective amount of pharmaceutical formulation of the present invention comprising an anti-C5 antigen-binding protein to the subject.

Antiphospholipid syndrome (APS) is an autoimmune disease characterized by arterial and venous thrombosis due to antiphospholipid antibodies. The disorder is referred to as primary when it occurs in the absence of another autoimmune disease. Secondary APS occurs in the context of an autoimmune disorder such as systemic lupus erythematosus. The catastrophic APS (CAPS) is a rare life-threatening form of APS in which widespread intravascular thrombosis results in multiorgan ischemia and failure. Thus, the present invention provides a method for treating or preventing APS (e.g., primary or secondary or CAPS) in a subject in need thereof comprising administering a therapeutically effective amount of anti-C5 antigen-binding protein (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4HI2169P; H4H12170P; H4H12171P; H4H12175P; H4H12176P2; H4H12177P2; H4H12183P2; H2M11682N; H2M11684N; H2M11694N; H2M11695N; ravulizumab, crovalimab or eculizumab) in a pharmaceutical formulation of the present invention to the subject. The present invention includes a method for reducing, stabilizing and/or eliminating one or more signs and/or symptoms of APS (e.g., primary, secondary or CAPS) in a subject suffering from APS and said signs and/or symptoms comprising administering a therapeutically effective amount of pharmaceutical formulation of the present invention comprising an anti-C5 antigen-binding protein to the subject.

Myasthenia gravis (MG) is a chronic autoimmune neuromuscular disease that causes weakness in the skeletal muscles, which are responsible for breathing and moving parts of the body, including the arms and legs. Thus, the present invention provides a method for treating or preventing myasthenia gravis in a subject in need thereof comprising administering a therapeutically effective amount of anti-C5 antigen-binding protein (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4HI2169P; H4H12170P; H4H12171P; H4H12175P; H4H12176P2; H4H12177P2; H4H12183P2; H2M11682N; H2M11684N; H2M11694N; H2M11695N; ravulizumab, crovalimab or eculizumab) in a pharmaceutical formulation of the present invention to the subject. The present invention includes a method for reducing, stabilizing and/or eliminating one or more signs and/or symptoms of MG in a subject suffering from MG and said signs and/or symptoms comprising administering a therapeutically effective amount of pharmaceutical formulation of the present invention comprising an anti-C5 antigen-binding protein to the subject.

Typical hemolytic uremic syndrome (tHUS) may follow a gastrointestinal infection with Shiga toxin-producing Escherichia coli (STEC). Typical HUS (STEC-HUS; Shiga toxin-producing Escherichia coli (STEC)-hemolytic uremic syndrome (HUS)) can be initiated when the Shiga toxin (or Shiga-like toxin), a known potent cytotoxin, binds to cell membrane glycolipid Gb3 (via domain B). Domain A is internalized and subsequently halts protein synthesis and induces apoptosis of the affected cell. The Shiga toxin has several additional effects on endothelial cells, one of which is enhanced expression of functional tissue factor that could contribute to microvascular thrombosis. The toxin causes damage to or activation of endothelium, red cells, and platelets. Thus, the present invention provides a method for treating or preventing tHUS in a subject in need thereof comprising administering a therapeutically effective amount of anti-C5 antigen-binding protein (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4HI2169P; H4H12170P; H4H12171P; H4H12175P; H4H12176P2; H4H12177P2; H4H12183P2; H2M11682N; H2M11684N; H2M11694N; H2M11695N; ravulizumab, crovalimab or eculizumab) in a pharmaceutical formulation of the present invention to the subject. The present invention includes a method for reducing, stabilizing and/or eliminating one or more signs and/or symptoms of tHUS in a subject suffering from tHUS and said signs and/or symptoms comprising administering a therapeutically effective amount of pharmaceutical formulation of the present invention comprising an anti-C5 antigen-binding protein to the subject.

The present invention also includes a method for switching therapeutic regimens for treating or preventing a C5-associated disease comprising ceasing administering a first such therapeutic regimen and administering a therapeutically effective amount of anti-C5 antigen-binding protein selected from H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; and H2M11695N in a pharmaceutical formulation of the present invention to the subject.

Certain standard treatments of C5-associated disease are burdensome and present significant dangers due to complications. The present invention also provides methods for avoiding such standard treatments and complications thereof by treating the underlying C5-associated disease (e.g., PNH or aHUS) with a pharmaceutical formulation of the present invention as set forth herein. For example, said standard treatments include blood transfusion, bone marrow transplantation (BMT), renal transplant, hemodialysis and/or balloon angioplasty.

Complications of blood transfusion include for example allergic reaction, fever, acute immune hemolytic reaction and blood-borne infection (e.g., human immunodeficiency virus (HIV), hepatitis C, hepatitis B and/or west nile virus). Thus, the present invention includes a method for avoiding blood transfusions and/or one or more complications of blood transfusions (e.g., allergic reaction, fever, acute immune hemolytic reaction and blood-borne infection) in a subject with a C5-associated disease (e.g., PNH or aHUS) by treating the underlying C5-associated disease (e.g., PNH or aHUS) with an pharmaceutical formulation of the present invention as set forth herein wherein treatment comprises administering a therapeutically effective amount of anti-C5 antigen-binding protein in a pharmaceutical formulation of the present invention to the subject.

Complications of bone marrow transplantation include for example graft-versus-host disease, stem cell (graft) failure, organ damage, infection, cataract, infertility and death. Thus, the present invention includes a method for avoiding bone marrow transplantation and/or one or more complications of bone marrow transplantation (e.g., graft-versus-host disease, stem cell (graft) failure, organ damage, infection, cataract, infertility and death) in a subject with a C5-associated disease (e.g., PNH or aHUS) by treating the underlying C5-associated disease (e.g., PNH or aHUS) with a pharmaceutical formulation of the present invention as set forth herein wherein treatment comprises administering a therapeutically effective amount of anti-C5 antigen-binding protein in a pharmaceutical formulation of the present invention to the subject.

Complications of hemodialysis include for example infection, sepsis, hypotension, muscle cramps, itching, sleep disturbance, sleep apnea, anemia, hypertension, fluid overload, pericarditis, hyperkalemia, amyloidosis or depression. Thus, the present invention includes a method for avoiding hemodialysis and/or one or more complications of hemodialysis (e.g., infection, sepsis, hypotension, muscle cramps, itching, sleep disturbance, sleep apnea, anemia, hypertension, fluid overload, pericarditis, hyperkalemia, amyloidosis or depression) in a subject with a C5-associated disease (e.g., PNH or aHUS) by treating the underlying C5-associated disease (e.g., PNH or aHUS) with a pharmaceutical formulation of the present invention as set forth herein wherein treatment comprises administering a therapeutically effective amount of anti-C5 antigen-binding protein in a pharmaceutical formulation of the present invention to the subject.

Complications of renal transplant include for example blood clots, bleeding, leaking from or blockage of the ureter, infection, kidney failure, kidney rejection, death, heart attack and stroke. Thus, the present invention includes a method for avoiding renal transplant and/or one or more complications of renal transplant (e.g., blood clots, bleeding, leaking from or blockage of the ureter, infection, kidney failure, kidney rejection, death, heart attack and stroke) in a subject with a C5-associated disease (e.g., PNH or aHUS) by treating the underlying C5-associated disease (e.g., PNH or aHUS) with an pharmaceutical formulation of the present invention as set forth herein wherein treatment comprises administering a therapeutically effective amount of anti-C5 antigen-binding protein in a pharmaceutical formulation of the present invention to the subject.

The pharmaceutical formulations of the present invention may be used to treat or prevent a C5-associated disease such as a C5-associated ophthalmologic disease, e.g., age-related macular degeneration (AMD; e.g., wet or dry), diabetic retinopathy (DR), non-infections uveitis, geographic atrophy, Stargardt Macular Dystrophy or optic neuritis.

AMD is the progressive degeneration of the macula (central part of the retina), typically, in people aged over 55 years. Various complement components, including C3, C5b-9, CFB, and CFH, have been detected in drusen as well as in AMD lesions. In addition, increased plasma levels of C3a, C3d, Bb, and C5a have been observed in AMD patients. These results suggest increased local and systemic complement activation in AMD.

DR is a progressive degeneration of retinal vasculature and neurons as a result of diabetes. Choriocapillaris of DR eyes contain significant levels of C3d and the C5b-9 complex. C5b-9 deposition may also be detectable in retinal vessels of patients with >9-year type-2 diabetes and increased C5a may be detected in the vitreous of patients with proliferative DR suggesting that complement activation is involved in retinal vascular damage in DR.

Non-infectious uveitis is inflammation—heat, redness, pain, and swelling—in one or both eyes which is not due to infection.

Geographic atrophy (GA) is a chronic progressive degeneration of the macula, as part of late-stage age-related macular degeneration (AMD). The disease is characterized by localized sharply demarcated atrophy of outer retinal tissue, retinal pigment epithelium and choriocapillaris. It typically starts typically in the perifoveal region and expands to involve the fovea with time, leading to central scotomas and permanent loss of visual acuity. It is bilateral in most cases.

Autosomal recessive Stargardt macular dystrophy (STGD1) is a dystrophy resulting from mutations in the ABCA4 (ABCR) gene. Mutations in ABCA4 also result in cone-rod dystrophy. The age of onset of juvenile and early adult STGD1 is usually 8-25 years with some cases occurring in older adults (lateadult onset STGD1). A hallmark of the disease is premature accumulation of lipofuscin (a brown-yellow autofluorescent pigment associated with aging) in the retinal pigment epithelia (RPE) of the eye, causing a pattern of yellowish flecks that extend outward from the macula.

Optic neuritis is an inflammation that damages the optic nerve. Pain and temporary vision loss in one eye are common symptoms of optic neuritis.

Thus, the present invention includes a method for treating or preventing a C5-associated ophthalmologic disease, e.g., age-related macular degeneration (AMD; e.g., wet or dry), diabetic retinopathy (DR), non-infections uveitis, geographic atrophy, Stargardt Macular Dystrophy or optic neuritis, in a subject in need thereof comprising administering, to the subject, a therapeutically effective amount of anti-C5 antigen binding protein, in a pharmaceutical formulation of the present invention, e.g., by intraocular or intravitreal injection.

The anti-C5 antigen-binding proteins of the present invention also reduce complement activity (e.g., C5-mediated complement activity) in the body of a subject. For example, in an embodiment of the invention, the complement activity is complement-mediated hemolysis (e.g., classical pathway mediated or alternative pathway mediated) or C5 activity (e.g., binding of C5a to C5aR1, generation of C5a and/or C5b from C5 precursor; or formation or deposition of membrane attack complex (MAC) in cells, e.g., endothelial cells). In an embodiment of the invention, complement activity is the capacity of serum taken from a subject's body to lyse sheep erythrocytes coated with anti-sheep antibodies. Thus, the present invention provides a method for reducing complement activity in the body of a subject comprising administering an anti-C5 antigen-binding protein, e.g., a therapeutically effective amount thereof, (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; H2M11695N; ravulizumab, crovalimab or eculizumab) in a pharmaceutical formulation of the present invention to the subject.

In certain embodiments, a therapeutically effective amount of anti-C5 antigen-binding protein in a pharmaceutical formulation of the invention is administered to a subject with a C5-associated disease. A therapeutically effective amount of anti-C5 antigen-binding protein in a pharmaceutical formulation of the present invention may vary depending upon the age and the size of a subject to be administered, target disease, conditions, route of administration, and the like. In an embodiment of the invention, a therapeutically effective amount of an anti-C5 antigen-binding protein in a pharmaceutical formulation of the present invention is about 0.1 to about 100 mg/kg body weight, about 5 to about 80, about 10 to about 70, or about 20 to about 50 mg/kg body weight (e.g., a single or multiple doses thereof). In an embodiment of the invention, a therapeutically effective amount of an anti-C5 antigen-binding protein in a pharmaceutical formulation of the present invention is about 0.1 mg to about 1000 mg, about 1 to about 600 mg, about 5 to about 500 mg, or about 10 to about 400 mg. Depending on the severity of the condition, the frequency and the duration of the treatment can be adjusted. In certain embodiments, the initial dose may be followed by administration of a second or a plurality of subsequent doses of the antigen-binding protein in an amount that can be approximately the same or less than that of the initial dose, wherein the subsequent doses are separated by at least 1 day to 3 days; at least one week, at least 2 weeks; at least 3 weeks or at least 4 weeks.

In an embodiment of the invention, a therapeutically effective amount of anti-C5 antigen-binding protein (e.g., pozelimab) in a pharmaceutical formulation of the invention is about 30 mg/kg body weight administered intravenously (IV) one or more times; optionally further including one or more doses of the formulation administered subcutaneously.

In an embodiment of the invention, a therapeutically effective amount of a further therapeutic agent that is an RNAi (e.g., cemdisiran) is about 0.001 to about 200.0 milligrams per kilogram body weight of the recipient per day, generally in the range of about 1 to 50 mg per kilogram body weight per day. For example, a therapeutically effective amount of RNAi, e.g., dsRNA (e.g., cemdisiran), is about 0.01 mg/kg, about 0.05 mg/kg, about 0.5 mg/kg, about 1 mg/kg, about 1.5 mg/kg, about 2 mg/kg, about 3 mg/kg, about 10 mg/kg, about 20 mg/kg, about 30 mg/kg, about 40 mg/kg, or about 50 mg/kg per single dose.

In a further embodiment of the invention, a further therapeutic agent that is administered to a subject in association with a pharmaceutical formulation of the invention. In an embodiment of the invention, the further therapeutic agent is administered at a dosage in accordance with the Physicians' Desk Reference 2003 (Thomson Healthcare; 57^{th} edition (Nov. 1, 2002)).

The present invention further provides methods for administering a pharmaceutical formulation of the present invention comprising an anti-C5 antigen-binding protein, e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4H12169P; H4H12170P; H4H12171P; H4H12175P; H4H12176P2; H4H12177P2; H4H12183P2; H2M11682N; H2M11684N; H2M11694N; H2M11695N; ravulizumab, crovalimab or eculizumab, to a subject, comprising introducing the pharmaceutical formulation into the body of the subject (e.g., a human), for example, parenterally. For example, the method comprises piercing the body of the subject with a needle of a syringe and injecting the pharmaceutical formulation into the body of the subject, e.g., into the vein, artery, eye, muscular tissue or subcutis of the subject.

The mode of administration of a pharmaceutical formulation of the present invention can vary. Routes of administration include parenteral, non-parenteral, oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, intraocular, intravitreal, transdermal or intra-arterial.

### Intravenous Administration

The anti-C5 antigen-binding proteins discussed herein (e.g., H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4H12168P; H4HI2169P; H4H12170P; H4H12171P; H4H12175P; H4H12176P2; H4H12177P2; H4H12183P2; H2M11682N; H2M11684N; H2M11694N; H2M11695N; ravulizumab, crovalimab or eculizumab) can be administered to a subject by an intravenous (IV) route. The present invention, thus, includes intravenous formulations that include an aqueous intravenous solution (e.g., NS) and a pharmaceutical formulation set forth herein. An intravenous formulation can be prepared by adding a pharmaceutical formulation that is set forth herein (e.g., about 200 mg/ml pozelimab; 20±4 mM histidine buffer; 100±20 mM L-arginine; 2±0.4% (w/v) sucrose; 0.15±0.075% (w/v) polysorbate-80; and water, pH 5.8±0.3) to an aqueous intravenous solution (e.g., NS). A pharmaceutical formulation can be added to an aqueous intravenous solution, for example, by injection through a medication port in the container holding the solution (e.g., a bag). The resulting intravenous formulation can then be administered to a subject. Intravenous formulations that are the result of such a method form part of the present invention along with methods of use thereof as set forth herein.

An aqueous intravenous solution, into which a pharmaceutical formulation can be introduced to produce an intravenous formulation, includes for example, 0.9% Normal Saline (NS, 0.9NaCl, or NSS), Lactated Ringers (LR, Ringers Lactate, or RL), Dextrose 5% in Water (D5 or D5W, an intravenous sugar solution); 0.45% Normal Saline (Half Normal Saline, 0.45NaCl, 0.45NS); 0.33% NaCl; 0.225% NaCl; 2.5% dextrose in water (D_{2.5}W); 3% NaCl; 5% NaCl; dextrose 5% in 0.45% NaCl (D₅ ½ NS); 5% dextrose and 0.45% NaCl; dextrose 5% in 0.9% NaCl (D₅NS); dextrose 5% in Lactated Ringer's (D₅LR); LR that contains 0.6% NaCl; 10% dextrose in water (D₁₀W); 20% dextrose in water (D20W); or 50% dextrose in water (D₅₀W). These solutions are well known in the art and are commercially available.

Vessels and other devices (e.g., sterile plastic or glass intravenous bottle or intravenous plastic bag) comprising such intravenous formulations also form part of the present invention.

Intravenous formulations can be administered into the veins of a subject by any of several methods known in the art. For example, the intravenous formulation can be administered by peripheral intravenous (PIV) line or a central IV line. A PIV introduces an intravenous formulation into the peripheral veins (typically, the veins in the arms, hands, legs and feet) of the subject. Central IV lines have catheters that are advanced through a vein and empty into a large central vein (a vein within the torso), usually the superior vena cava, inferior vena cava or even the right atrium of the heart.

Thus, the present invention provides a method for intravenously administering, to a subject, an intravenous formulation of the present invention which includes a pharmaceutical formulation set forth herein (e.g., about 200 mg/ml pozelimab; 20±4 mM histidine buffer; 100±20 mM L-arginine; 2±0.4% (w/v) sucrose; 0.15±0.075% (w/v) polysorbate-80; and water, pH 5.8±0.3) in an aqueous intravenous solution (e.g., NS) comprising introducing the intravenous formulation into a vein (e.g., a peripheral vein) of the subject, e.g., by IV infusion (e.g., drip infusion or pump infusion). Also provided are methods for administering an intravenous formulation of the present invention which may include the step of adding a pharmaceutical formulation set forth herein (e.g., about 200 mg/ml pozelimab; 20±4 mM histidine buffer; 100±20 mM L-arginine; 2±0.4% (w/v) sucrose; 0.15±0.075% (w/v) polysorbate-80; and water, pH 5.8±0.3) to an aqueous intravenous solution (e.g., NS) and introducing the resulting intravenous formulation into a vein of the subject, e.g., by IV infusion (e.g., drip infusion or pump infusion). Such methods optionally comprise further administering a pharmaceutical formulation by a route other than intravenously, e.g., subcutaneously.

The present invention includes methods for treating or preventing a C5-associated disease (e.g., PNH) in a subject by administering an intravenous formulation comprising intravenously administering anti-C5 antigen-binding protein to the subject (e.g., one or more IV doses of 1, 3, 10, 15 or 30 mg/kg). Optionally, the method further includes the step of administering a pharmaceutical formulation of the present invention to the subject by a route other than IV, for example, subcutaneously, i.e., wherein the pharmaceutical formulation is administered without the step of forming an intravenous formulation.

### EXAMPLES

The following example is provided to further describe the present invention and should not be construed as a limitation thereof. The scope of the present invention includes any of the methods and pharmaceutical formulations which are set forth below in the following examples.

### Example 1: Conformational Stability of H4H12166P

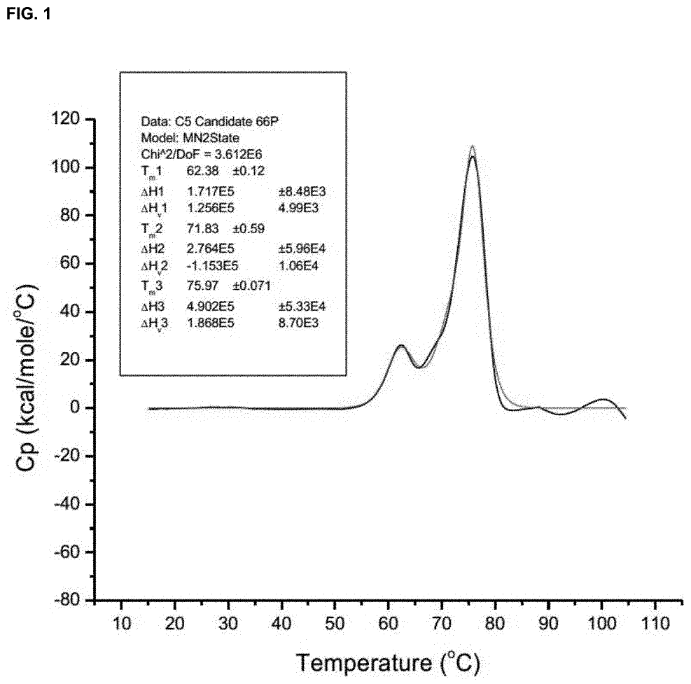

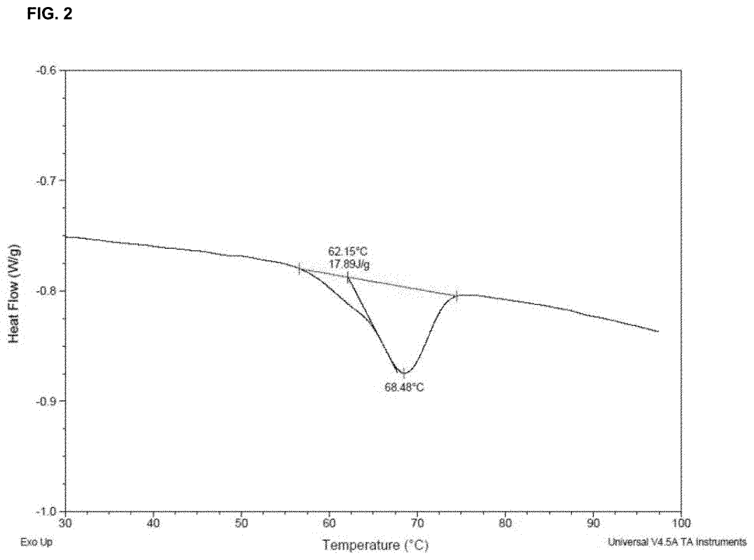

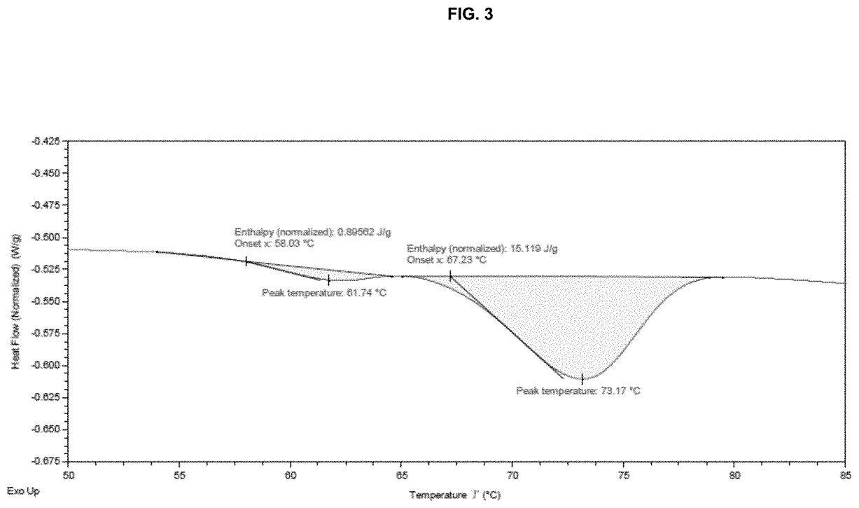

The melting temperatures (Tₘ) of H4H12166P were determined by TA-differential scanning calorimetry (DSC) and VP-DSC using the parameters set forth in Tables 1-1 and 1-2. Melting temperatures that were determined are summarized in Table 1-3. In addition, the thermal melting profiles of H4H12166P determined by VP-DSC at 1 mg/mL and by TA-DSC at 150 mg/mL and 200 mg/mL are shown in FIG. 1, FIG. 2 and FIG. 3, respectively.

TABLE 1-1
Method Parameters for VP-DSC*

| Parameter | Value | |
|---|---|---|
| Initial Temperature | 10° | C. |
| Equilibration Time at | 15 | min |
| Initial Temperature | | |
| Scan Rate | 90° C. per hour | |
| | (1.5° C. per minute) | |
| Final Temperature | 105° | C. |

*MicroCal VP-Differential Scanning Calorimeter

TABLE 1-2
Method Parameters for TA-DSC*

| Step | Action |
|---|---|
| 1 | Equilibrate at 10° C. |
| 2 | Modulate +/−0.5° C. every 60 seconds |
| 3 | Isothermal for 5 minutes |
| 4 | Ramp 2° C. per minute to 102° C. |

*TA Instruments Differential Scanning Calorimeter

TABLE 1-3
H4H12166P melting temperatures determined by DSC

| Method | Sample Composition | Tₘ1 Onset | | Tₘ1 | Tₘ2 | |
|---|---|---|---|---|---|---|
| VP-DSC | 1 mg/mL H4H12166P, | 52° C.-55° C. | | 62.4° C. | 75.9° | C.* |
| | 10 mM histidine, pH 5.5 | | | | | |
| TA-DSC | 150 mg/mL H4H12166P, | ~55° | C. | ~61° C. | 68.5° | C. |
| | 10 mM histidine, pH 5.5 | | | (not well | | |
| | | | | defined) | | |
| TA-DSC | 200 mg/mL H4H12166P, | 58.0° | C. | 61.7° C. | 73.2° | C. |
| | 20 mM histidine, pH 5.8, | | | | | |
| | 100 mM arginine-HCl, | | | | | |
| | 2% (w/v) sucrose, 0.15% | | | | | |
| | (w/v) polysorbate 80 | | | | | |

*The fitted model showed 3 transitions in the VP-DSC unfolding profile and the 75.9° C. represents the Tₘ of the 3^{rd} peak, however, since the second peak was not well resolved it is not included in the table and 75.9° C. is designated as Tₘ2.

The first transition (Tₘ1) in the DSC thermograms observed at lower temperatures most likely represents the thermal unfolding of the CH2 domain in the Fc region, whereas the main transition (larger endotherm) is from the domains of the Fab region. The Tₘ1 values determined for all samples are similar (61-62° C.), although this peak is not well-defined in the TA-DSC thermogram for the 150 mg/mL H4H12166P (FIG. 2). The measured Tₘ2 value, corresponding to the main transition, in the 1 mg/mL H4H12166P DSC thermogram (˜76° C.) is higher compared to the main transition temperatures in the 150 (69° C.) and 200 (73° C.) mg/mL H4H12166P sample. These differences between the Tₘs determined by VP-DSC and TA-DSC may be attributed to differences in instrumentation and data analysis, in addition to the large difference in protein concentration in the respective samples. The slightly higher Tₘ2 value determined for the 200 mg/mL H4H12166P sample as compared to the 150 mg/mL H4H12166P sample may be due to (1) the 0.3 pH unit difference between the two formulations, and (2) the presence of formulation stabilizers in the 200 mg/mL H4H12166P sample.

### Example 2: Effect of pH, Temperature and Headspace

Temperature, pH and container headspace were evaluated in a Design of Experiment (DoE) study to characterize the degradation pathways of 150 mg/mL H4H12166P. A risk assessment was completed. The factors used in this study are shown in Table 2-1.

TABLE 2-1
Controllable Factors and Levels/Values

| Factors | Type of factor | Values | | |
|---|---|---|---|---|
| pH | Continuous | 5.7 | 6.2 | 6.8 |
| Temperature | Continuous | 25 | 37 | 45 |
| (° C.) | | | | |
| Air Headspace | Continuous | 2 | 5.5 | 11.5 |
| Volume (mL) | | | | |

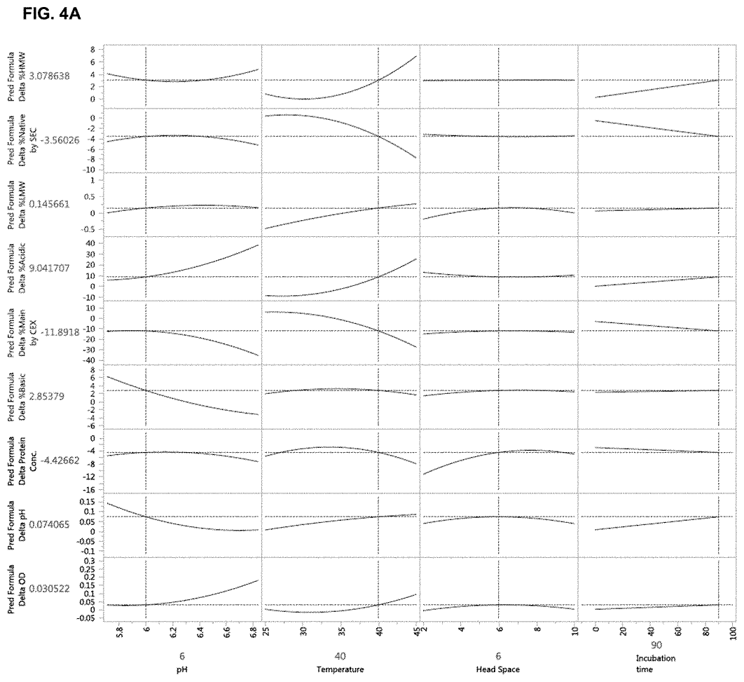

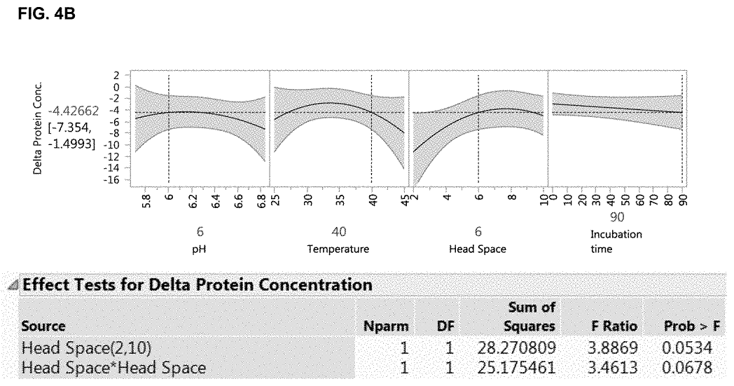

Results from the study, designed to characterize 150 mg/mL H4H12166P in 20 mM histidine buffer over a range of temperatures, pH and container headspaces, is illustrated in FIG. 4. The impact of these factors on the evaluated quality attributes of H4H12166P are summarized below:
Temperature and pH showed the largest impact on H4H12166P quality attributes such as molecular size and charge variants, pH and turbidity.
   Formation of molecular size variants (HMW species) and acidic charge variants increased with temperature with a corresponding decrease in the % Main peaks, determined by SE-UPLC and CEX-UPLC, respectively.
   Formation of basic charge variants increased progressively with decreasing pH at higher temperatures.
   At higher temperatures, a larger increase in solution pH (by up to 0.15 pH units) was observed following incubation for 90 days at pH 5.7 as compared to pH 6.7.
   An increase in turbidity was measured at higher temperature and pH.

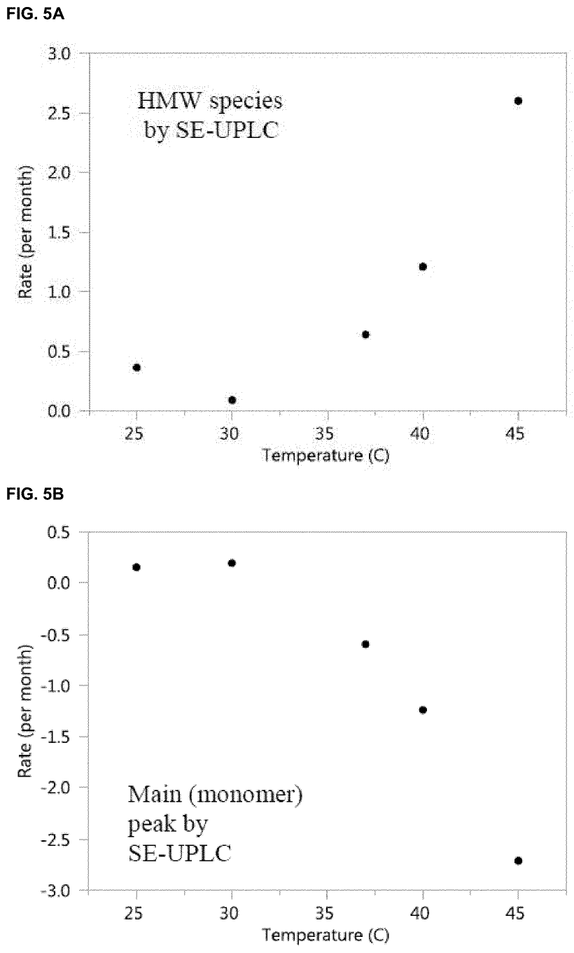

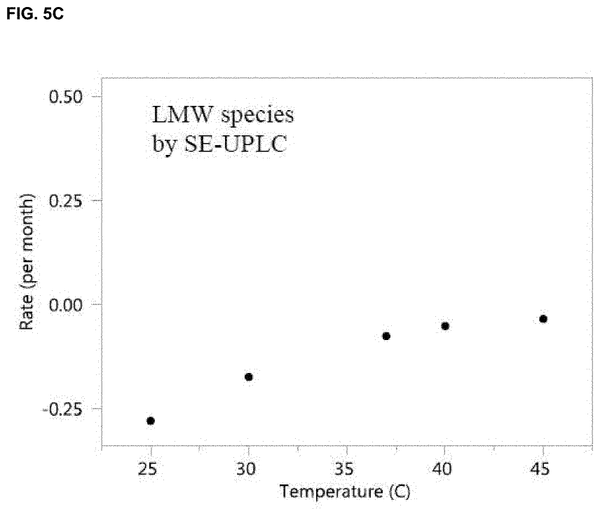

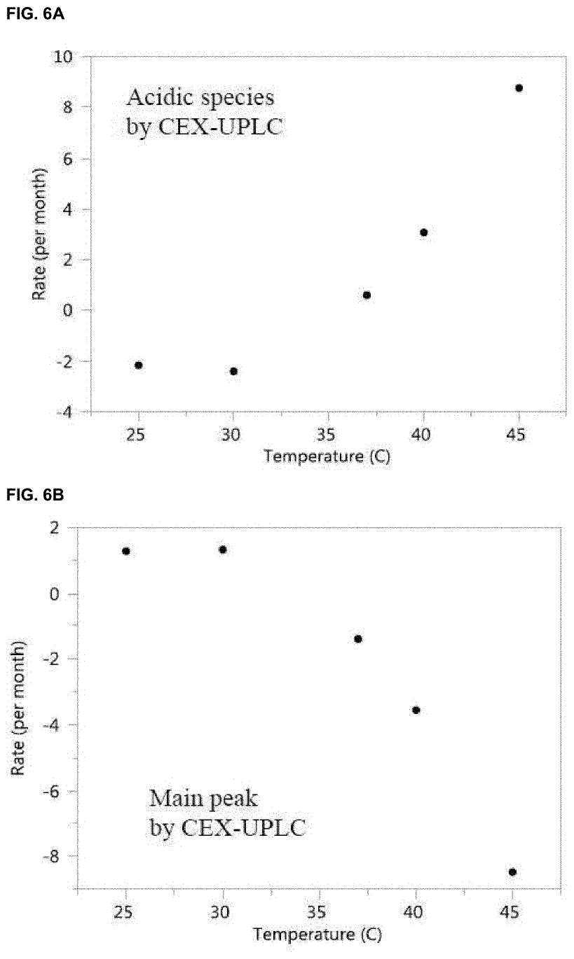

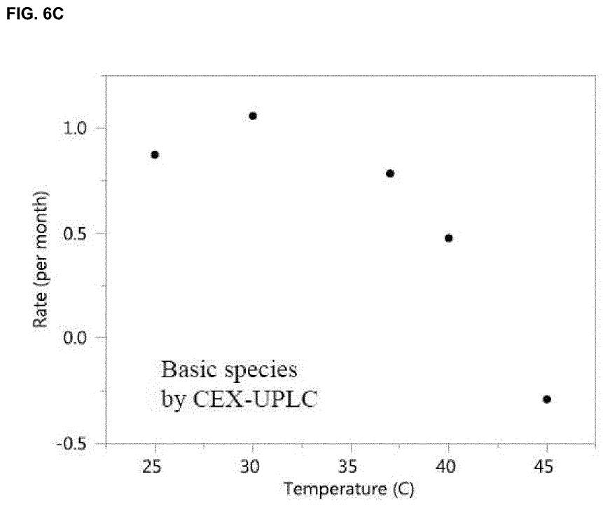

The transfer functions obtained from analysis of the DoE study results were used to estimate the rates of formation of molecular size and charge variant species in 150 mg/mL H4H12166P, 20 mM histidine, pH 5.8, at temperatures ranging from 25° C. to 45° C. (Table 2-2); these rates are plotted in FIG. 5 and FIG. 6, respectively, as a function of temperature, as graphical representations of degradation rates at different temperatures. A summary of these results is provided below:
Formation of high molecular weight species (HMW) and acidic charge variants are the dominant degradation pathways in H4H12166P following thermal stress at
   37° C. (HMW rate=0.6% per month and Acidic rate=0.6% per month),
   40° C. (HMW rate=1.2% per month and Acidic rate=3.1% per month), and
   45° C. (HMW rate=2.6% per month and Acidic rate=8.8% per month).
Minimal fragmentation (LMW species) or formation of basic charge variants were observed under these conditions.
At temperatures higher than 30° C., the rates of degradation (% HMW and Acidic) increase almost linearly up to 45° C.
The rate of HMW formation at 30° C. was lower as compared to 25° C. (Table 2-2), however, the magnitude of this difference (0.27%) is smaller than the error in the model fit (0.45%) and is not meaningful.
A decreasing trend was observed in the rate of change in basic charge variant species with increasing temperature (FIG. 6), which may be due to their conversion into acidic charge variant species. Nonetheless, the overall change (<1%) is smaller than the error in the model fit (1.5%) so the changes may not be
   meaningful within the parameters evaluated in this study.

TABLE 2-2
Rates Of Change of 150 mg/ml H4H12166P Molecular Size
and Charge Variant Species at Different Temperatures

| | Rate @ | Rate @ | Rate @ | Rate @ | Rate @ |
|---|---|---|---|---|---|
| | 25° C. | 30° C. | 37° C. | 40° C. | 45° C. |
| | (per | (per | (per | (per | (per |
| Attribute | month) | month) | month) | month) | month) |
| % HMW | 0.3628 | 0.0906 | 0.6409 | 1.2095 | 2.6007 |
| by SEC | | | | | |
| % Monomer | 0.1556 | 0.1954 | −0.5959 | −1.2376 | −2.7106 |
| by SEC | | | | | |
| % LMW | −0.2783 | −0.1732 | −0.0754 | −0.0512 | −0.0344 |
| by SEC | | | | | |
| % Acidic | −2.1582 | −2.3911 | 0.6027 | 3.0715 | 8.7670 |
| by CEX | | | | | |
| % Main | 1.2809 | 1.3268 | −1.3923 | −3.5518 | −8.4764 |
| by CEX | | | | | |
| % Basic | 0.8740 | 1.0586 | 0.7841 | 0.4761 | −0.2909 |
| by CEX | | | | | |

The rationale for including headspace as one of the factors in this characterization study was to evaluate the impact of different amounts of headspace oxygen on the susceptibility of H4H12166P to undergo methionine oxidation. Methionine oxidation was quantified by peptide mapping (LCMS), however, since this is not a high throughput method the test was performed only on the 150 mg/mL H4H12166P, 20 mM histidine, pH 6.2 sample filled in a 10 mL glass vial (largest headspace) at the t=0 and 45° C. 28 day time point, as a worst case scenario. The results, listed in Table 2-3, show only a very small increase (1.3%) in oxidation of Met105, which is in the CDR of H4H12166P, under these incubation conditions. The other Met residues showed lower and negligible changes in oxidation upon thermal incubation. Container headspace showed a minimal impact on other monitored quality attributes over the evaluated range (2.5 mL to 11.5 mL) (FIG. 4). The lower protein recovery (delta protein concentration) observed at smaller headspaces is not statistically significant as shown by the large confidence intervals and Effect Tests (FIG. 4). Thus, oxidation of H4H12166P due to the presence of headspace oxygen is not considered to be a degradation pathway.

TABLE 2-3
Methionine Oxidation, Determined by Peptide Mapping, in 150
mg/ml H4H12166P in 20 mM Histidine, pH 6.2 Filled in 10 ml
Glass Vial (Largest Headspace) following Thermal Stress

| Incubation | HC Met105 | HC | HC | LC |
|---|---|---|---|---|
| Condition | (CDR) | Met252 | Met428 | Met4 |
| t = 0 | 4.8 | 3.3 | 1.0 | 1.1 |
| 45° C. 28 days | 6.1 | 4.1 | 1.2 | 1.1 |

### Example 3: Effect of Freeze/Thaw, Agitation and Forced Oxidation (Hydrogen Peroxide Spiking)

The susceptibility of 150 mg/mL H4H12166P in 20 mM histidine, pH 6.2 to freeze/thaw, agitation and oxidation (H₂O₂ spiking) stress was evaluated using a single factor study design. The incubation/stress conditions used to assess the stability of H4H12166P are shown in Table 3-1. The concentrations of hydrogen peroxide evaluated in this study are listed in Table 3-2.

TABLE 3-1
Summary of Stress/Incubation Conditions

| Storage/Stress | Incubation/ | |
|---|---|---|
| Condition | Stress Time | Conditions |
| Oxidation* | 0, 2, 6, 24 hours | 37° C. |
| Freeze/thaw | 0, 4 and 8 cycles | Each cycle included −30° C. |
| | | freeze for at least 2 hours |
| | | and room temperature thaw |
| Agitation (250 rpm | 0, 6 and 24 hours | Room temperature |
| on orbital shaker) | | (~22° C.) |

*Following incubation with H₂O₂, all samples were immediately buffer exchanged into 20 mM histidine, pH 6.2 buffer using Millipore spin columns to quench oxidation during sample analysis.

TABLE 3-2
Hydrogen Peroxide Levels Used in the Forced Oxidation Study

| | Hydrogen Peroxide | Hydrogen Peroxide |
|---|---|---|
| Formulation # | Concentration (ppm) | Concentration (%) |
| F1 | 0 | 0 |
| F2 | 0.01 | 0.000001 |
| F3 | 0.1 | 0.00001 |
| F4 | 0.5 | 0.00005 |
| F5 | 1 | 0.0001 |
| F6 | 10 | 0.001 |
| F7 | 100 | 0.01 |
| F8 | 500 | 0.05 |

Fourteen (14) mL of F1 (control) and ten (10) mL of each subsequent formulation (F2 to F8) listed in Table 3-2 was prepared. Each formulation was filter-sterilized, using a syringe and Millipore Millex GV (PVDF Durapore) 0.2 μM filter, in a laminar flow hood. Formulations F2 to F8 were spiked with H₂O₂ in a laminar flow hood after being filter-sterilized. The formulations were dispensed as follows:

1. One (1) mL of formulation F1 was filled in ten (10) 2 mL glass vials (also used for F/T and agitation tests).
2. One (1) mL of formulations F2 to F8 were each filled in four (4) 2 mL glass vials.
The analytical testing plan for the F/T, agitation and oxidation stressed samples is shown in Table 3-4.

TABLE 3-4
Analytical Plan for F/T, Agitation and Oxidation (H₂O₂ Spiking)

| Assay | Sample Analyzed |
|---|---|
| Visual appearance | All samples |
| pH | All samples |
| Turbidity (OD@405 nm) | All samples |
| Protein recovery by RP-UPLC | All samples |
| Molecular size variants by | All samples |
| SEC-UPLC | |
| Charge variants by CEX-UPLC | All samples |
| Met Oxidation | t = 0; |
| by Peptide | 0 ppm H₂O₂ 37° C. 24 hrs; |
| Mapping (LC-MS) | 1 ppm H₂O₂ 37° C. 24 hrs; |
| | 500 ppm H₂O₂ 37° C. 24 hrs |
| Met Oxidation by HIC-HPLC | All samples |
| Potency | t = 0; |
| | 0 ppm H₂O₂ 37° C. 24 hrs; |
| | 1 ppm H₂O₂ 37° C. 24 hrs; |
| | 500 ppm H₂O₂ 37° C. 24 hrs |

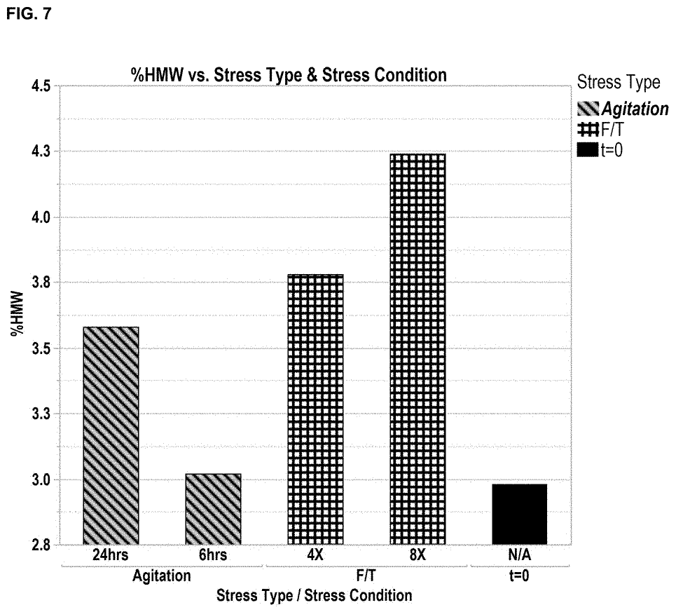

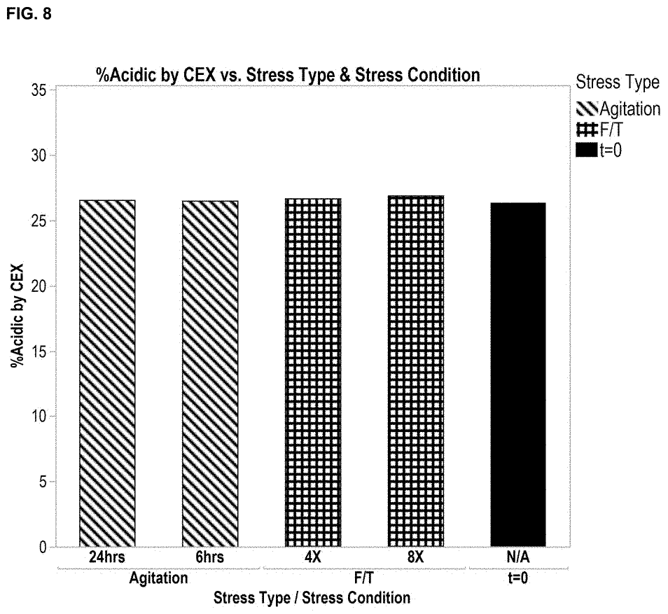

After being subjected to freeze/thaw and agitation stress, under the conditions described in Table 3-1, 150 mg/mL H4H12166P, 20 mM histidine, pH 6.2 showed increased levels of HMW species (FIG. 7).
Following 4× and 8× freeze/thaw cycles, the HMW species increased by 0.8% and 1.3%, respectively.
A minimal increase in HMW species was observed following 6 hours of agitation, and >0.5% increase in HMW species was observed following 24 hours of agitation.

No changes in charge variants, determined by CEX-UPLC, were observed following freeze/thaw or agitation stress (see FIG. 8). The levels of methionine oxidation in 150 mg/mL H4H12166P following incubation with different concentrations of hydrogen peroxide at 37° C. for 24 hours were determined by peptide mapping (LC-MS). No increase in methionine oxidation or significant loss of potency was observed in H4H12166P when incubated with 1 ppm hydrogen peroxide (Table 3-5), and an ˜80% increase in Met105 oxidation, ˜21% increase in Met252 oxidation and ˜6% increase in Met428 oxidation was observed when H4H12166P was incubated with 500 ppm hydrogen peroxide. A significant loss of potency, determined by bioassay, was observed following incubation with 500 ppm hydrogen peroxide.

TABLE 3-5
Methionine Oxidation Levels Determined by Peptide Mapping
and Corresponding Relative Potencies of 150 mg/ml H4H12166P
following Incubation with Hydrogen Peroxide

| | % Oxidation by Peptide Mapping | | | | |
|---|---|---|---|---|---|
| Stress | HC Met105 | HC | HC | LC | Potency* |
| Conditions | (CDR) | Met252 | Met428 | Met4 | (Bioassay) |
| 0 ppm H₂O₂ | 4.3 | 3.4 | 0.8 | 1 | 117% |
| t = 0 | | | | | |
| 0 ppm H₂O₂ | 4.2 | 3.5 | 1.2 | 0.9 | 89% |
| 37° C. 24 hrs | | | | | |
| 1 ppm H₂O₂ | 4.2 | 3.6 | 1.1 | 0.7 | 85% |
| 37° C. 24 hrs | | | | | |
| 500 ppm H₂O₂ | 82.9 | 25.1 | 7.7 | 1.1 | 31% |
| 37° C. 24 hrs | | | | | |

*Relative potency = IC50 (Ref Std/Sample) * 100. In the bioassay, anti-C5 (REGN3918) inhibition of complement dependent cytotoxicity (complement-dependent cytotoxicity (CDC)) mediated cell death was determined. C5 is cleaved into C5a and C5b which promotes initiation and progression of the CDC pathway and formation of the MAC complex that facilitates cell death. The readout for this assay was cell death measured by the CytoTox Glo kit (Promega). The kit utilizes a substrate that luminescences when cleaved by enzymes released from dead cells; therefor it can measure the relative amount of cell death in a well.

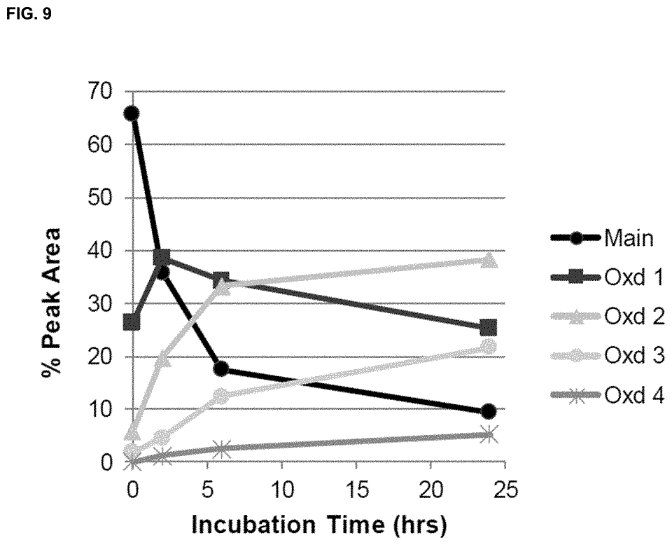

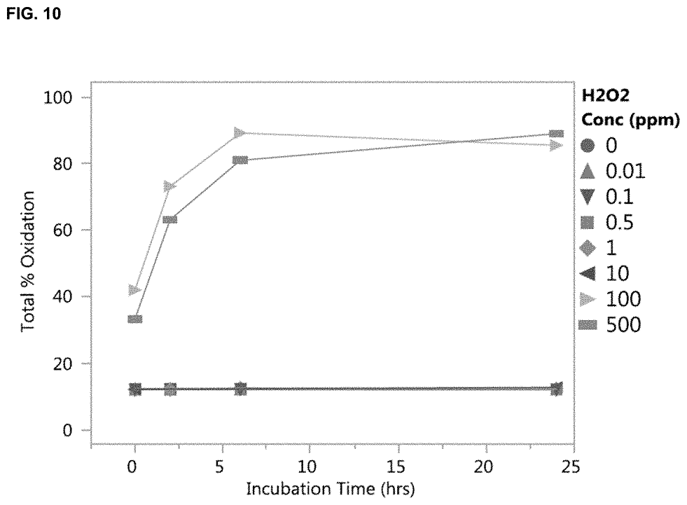

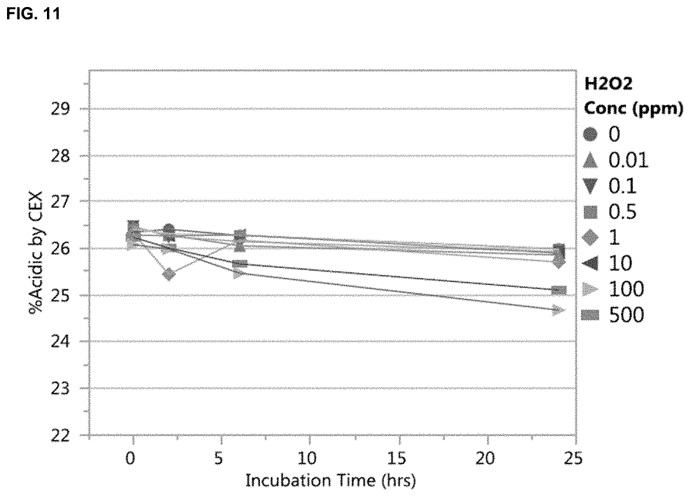

A high throughput hydrophobic interaction chromatography (HIC) HPLC-based method was developed to quantify the levels of methionine oxidation in H4H12166P. This method is not sufficiently sensitive to allow identification of specific methionine residues that undergo oxidation, but distinct peaks, that increased with incubation time, were resolved in the chromatogram. The peak areas for the 500 ppm hydrogen peroxide sample, which represent the change in different oxidized species, are shown in FIG. 9. The total oxidation (calculated from the sum of all peak areas for oxidized species, excluding the main peak) at different concentrations of hydrogen peroxide following incubation at 37° C. for up to 24 hours are shown in FIG. 10. These results show undetectable oxidation in H4H12166P when incubated with up to 10 ppm hydrogen peroxide under these conditions. The results for 1 ppm and 500 ppm hydrogen peroxide obtained by peptide mapping (LC-MS) and HIC-HPLC are qualitatively consistent (Table 3-5 and FIG. 10).

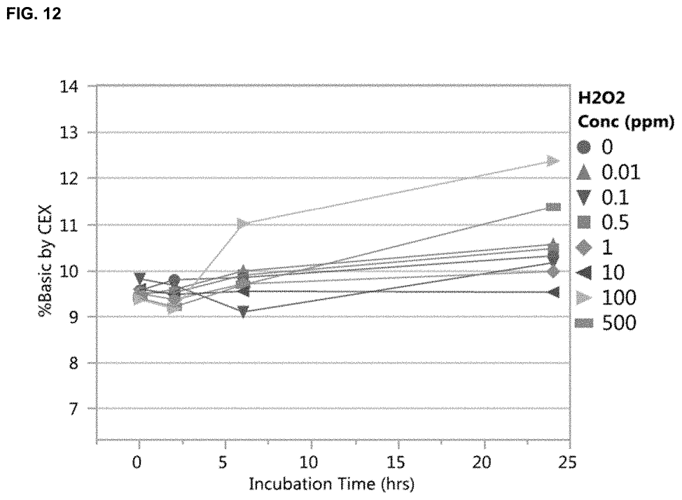

The H4H12166P samples incubated with hydrogen peroxide were also analyzed for charge variants (CEX-UPLC) and molecular size variants (SE-UPLC). A minimal (˜1%) decrease in acidic charge variants and a comparable increase in basic charge variants was observed only at 100 and 500 ppm hydrogen peroxide (FIG. 11 and FIG. 12).

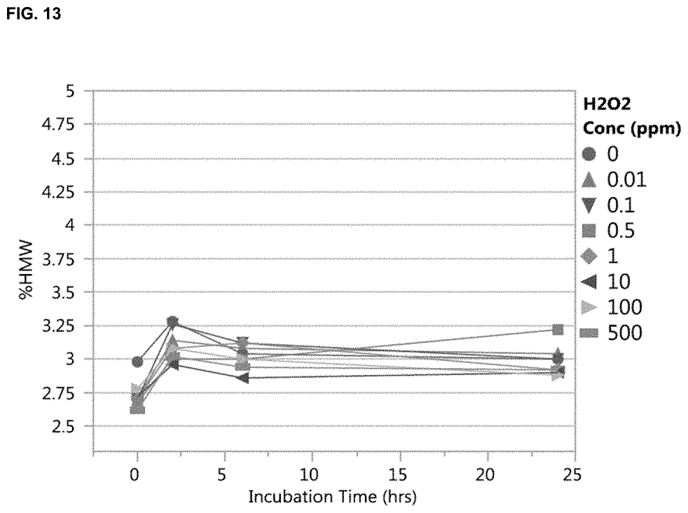

There was no meaningful increase in formation of HMW species even at the highest tested concentration of hydrogen peroxide (FIG. 13) indicating that even high levels of H4H12166P methionine oxidation do not generate conformational species that are prone to self-association or aggregation.

### Example 4: Comparison of Viscosities of H4H12166P and Comparator Molecules at High Concentrations

Viscosity was measured using a Rheosense m-VROC viscometer. Prior to analysis, standards and formulations were filtered through a 0.22 μm PVDF spin filter. Two standards of known viscosities were measured before the unknown sample viscosity analysis: 2 cP and glycerol standards. Viscosity measurements were performed at 20° C.

TABLE 4-1
Viscosity of H4H12166P and various anti-C5 antibodies ^{#}

| | | Viscosity at 20° C. (cps) | | | | | |
|---|---|---|---|---|---|---|---|
| | Arginine HCl | 150 | 175 | 200 | 211 | 220 | 242 |
| Antibody | Concentration | mg/mL | mg/mL | mg/mL | mg/mL | mg/mL | mg/mL |
| Pozelimab | 0 | 9.4 | 12.4 | 24.6 | 27.4 | 34.7 | 63.5 |
| (H4H12166P) | 50 | — | 8.3 | 14.3 | 19.2 | 24.1 | 42.3 |
| Tesidolumab | 0 | 124 | too | too | | | |
| (SEQ ID NOs: | | | high | high | | | |
| 362 and 363) | 50 | 24 | 48 | 91 | | | |
| Eculizumab | 0 | 17.8 | N/A | N/A | | | |
| (SEQ ID NOs: | 50 | 9.8 | 22.4 | 38.6 | | | |
| 364 and 365) | | | | | | | |
| Ravulizumab′ | 0 | 67.8 | too | too | | | |
| (SEQ ID NOs: | | | high | high* | | | |
| 366 and 367) | 50 | 22.3 | 50.5 | 117.8 | | | |

^{#} All molecules were analyzed in 10 mM histidine, pH 5.5.
*Sample stored at 5° C. turned into a cloudy gel (probably due to low solubility).
′C-terminal lysine included

### Example 5: Long-Term and Accelerated Stability Testing of 200 mg/ml H4H12166P

Stability studies evaluated the stability of a liquid, aqueous H4H12166P 200 mg/mL formulation under long term storage and stress conditions. Two stability studies were initiated:

(1) 0.5 mL fill in a 2 mL type 1 borosilicate glass vial stored in the upright orientation, and
(2) 2.5 mL fill in a 5 mL type 1 borosilicate glass vial stored in the inverted orientation

TABLE 5-1
Study Design

| | | 2 mL Glass Vial, | 5 mL Glass Vial, |
|---|---|---|---|
| | | Stored Upright | Stored Inverted |
| Storage | Condition | 5° C. | 5° C. |
| | Duration | 0, 1, 3, 6, 9, 12, 18, | 0, 1, 3, 6, 9, 12, 18, |
| | | 24, and 36 months | 24, and 36 months |
| Accelerated | Condition | 25° C./60% RH | 25° C./60% RH |
| | Duration | 0, 0.5, 1, 3, 6 months | 0, 1, 3, 6 months |
| Thermal | Condition | 40° C./75% RH | 40° C./75% RH |
| Stress | Duration | 0, 0.25, 0.5, 1, 2 and | 0, 0.5, 1 and 3 |
| | | 3 months | months |

### Results

The attributes of an aqueous formulation comprising 200 mg/mL H4H12166P, 20 mM histidine, pH 5.8, 100 mM L-arginine hydrochloride, 2% (w/v) sucrose, 0.15% (w/v) polysorbate 80, following 6 months of long term (5° C.) storage in 2 mL Schott Type 1 borosilicate glass vials (stored upright), are set forth in Table 5-2. Table 5-3 and Table 5-4 summarizes the attributes of liquid, aqueous H4H12166P 200 mg/mL formulation after storage at accelerated condition of 25° C., 60% RH and at stressed conditions of 40° C., 75% RH, respectively.

TABLE 5-2
Long-term Stability at 5° C.^{#}

| | | | 1 M | | | | |
|---|---|---|---|---|---|---|---|
| Assay | | t = 0 | (months) | 3 M | 6 M | 9 M | 12 M |
| Appearance | | ⁺PASS | ⁺PASS | ⁺PASS | ⁺PASS | ⁺PASS | ⁺PASS |
| | | | No change | No change | No change | No change | No change |
| | | | in clarity | in clarity | in clarity | in clarity | in clarity |
| | | | or turbidity | or turbidity | or turbidity | or turbidity | or turbidity |
| pH | | 5.7 | 5.8 | 5.8 | 5.8 | 5.8 | 5.8 |
| Total Protein by Solo-VPE (mg/mL) | | 212 | 210 | 208 | 214 | 192 | 205 |
| Turbidity (increase in OD at 405 nm) | | — | 0.00 | 0.00 | 0.01 | 0.00 | 0.01 |
| % Relative Potency by Bioassay | | 118 | N/A | N/A | N/A | NR | 111 |
| Purity | % HMW | 0.9 | 0.9 | 1.0 | 1.1 | 1.1 | 1.2 |
| by | % Main | 98.9 | 98.8 | 98.8 | 98.7 | 98.3 | 98.3 |
| SE-UPLC | % LMW | 0.2 | 0.4 | 0.2 | 0.3 | 0.6 | 0.5 |
| Purity by | % Purity | 93.1 | 93.4 | 91.9 | 92.1 | 91.6 | 92.3 |
| Reduced | % LMW | 0.7 | 0.4 | 1.7 | 1.3 | 1.5 | 0.9 |
| MCE-SDS | % NGHC | 5.2 | 5.4 | 4.9 | 5.1 | 4.3 | 5.4 |
| Purity | % Main Peak | 98.1 | 98.0 | 95.8 | 97.2 | 98.3 | 95.7 |
| by Non- | Purity | | | | | | |
| Reduced | % LMW | 2.0 | 2.0 | 3.5 | 2.4 | 1.6 | 4.2 |
| MCE-SDS | % HMW | 0.0 | 0.0 | 0.6 | 0.4 | 0.1 | 0.1 |
| Charge Variant | % Region 1 | 28.8 | 30.4 | 30.3 | 30.3 | 32.8 | 31.8 |
| Analysis | % Region 2 | 60.2 | 59.0 | 58.6 | 55.9 | 56.0 | 57.1 |
| by iCIEF | % Region 3 | 11.0 | 10.7 | 11.1 | 13.8 | 11.2 | 11.1 |
| Particulate Matter | 2-10 μm | 6908 | N/A | 10918 | 983 | NR | 800 |
| (Micro-Flow Imaging; | ≥10 μm | 50 | N/A | 13 | 8 | NR | 43 |
| particles/mL) | ≥25 μm | 6 | N/A | 1 | 0 | NR | 5 |

^{#}USP Type 1 clear glass, 2 mL vial with 13 mm gray chlorobutyl rubber stopper with FluroTec. stored upright and containing 200 mg/mL H4H12166P, 20 mM histidine, pH 5.8, 100 mM L-arginine hydrochloride, 2% (w/v) sucrose, 0.15% (w/v) polysorbate 80
⁺Pass = Clear to pale yellow, and essentially free from visible particulates.

TABLE 5-3
Stability under Accelerated Conditions of 25° C., 60% RH*

| Assay | | t = 0 | 0.5 M | 1 M | 3 M | 6 M |
|---|---|---|---|---|---|---|
| Appearance | | ⁺PASS | ⁺PASS | ⁺PASS | ⁺PASS | ⁺PASS |
| | | | No change | No change | No change | No change |
| | | | in clarity | in clarity | in clarity | in clarity |
| | | | or turbidity | or turbidity | or turbidity | or turbidity |
| pH | | 5.7 | 5.8 | 5.8 | 5.8 | 5.8 |
| Total Protein by Solo-VPE (mg/mL) | | 212 | 211 | 209 | 208 | 212 |
| Turbidity (increase in OD at 405 nm) | | 0.00 | 0.00 | 0.01 | 0.01 | 0.03 |
| % Relative Potency by Bioassay | | 118 | N/A | N/A | N/A | N/A |
| Purity | % HMW | 0.9 | 1.1 | 1.1 | 1.3 | 1.5 |
| by | % Main | 98.9 | 98.6 | 98.6 | 98.4 | 98.1 |
| SE-UPLC | % LMW | 0.2 | 0.3 | 0.3 | 0.3 | 0.4 |
| Purity by | % Purity | 93.1 | 93.0 | 93.4 | 91.6 | 90.8 |
| Reduced | % LMW | 0.7 | 0.6 | 0.6 | 1.2 | 2.3 |
| MCE-SDS | % NGHC | 5.2 | 5.3 | 5.3 | 5.6 | 5.3 |
| Purity | % Main Peak | 98.1 | 98.0 | 97.8 | 95.7 | 96.6 |
| by Non- | Purity | | | | | |
| Reduced | % LMW | 2.0 | 2.0 | 2.2 | 3.6 | 2.7 |
| MCE-SDS | % HMW | 0.0 | 0.0 | 0.0 | 0.7 | 0.7 |
| Charge Variant | % Region 1 | 28.8 | N/A | 28.9 | 31.0 | 33.1 |
| Analysis by | % Region 2 | 60.2 | N/A | 56.9 | 53.5 | 47.2 |
| iCIEF | % Region 3 | 11.0 | N/A | 14.2 | 15.6 | 19.7 |
| Particulate Matter | 2-10 μm | 6908 | N/A | N/A | N/A | 2117 |
| (Micro-Flow Imaging; | ≥10 μm | 50 | N/A | N/A | N/A | 41 |
| particles/mL) | ≥25 μm | 6 | N/A | N/A | N/A | 7 |

*USP Type 1 clear glass, 2 mL vial with 13 mm gray chlorobutyl rubber stopper with FluroTec. stored upright and containing 200 mg/mL H4H12166P, 20 mM histidine, pH 5.8, 100 mM L-arginine hydrochloride, 2% (w/v) sucrose, 0.15% (w/v) polysorbate 80.
⁺Pass = Clear to pale yellow, and essentially free from visible particulates.

TABLE 5-4
Stability under Accelerated Conditions of 40° C., 75% RH^{~}

| Assay | | t = 0 | 0.25 M | 0.5 M | 1 M | 2 M | 3 M |
|---|---|---|---|---|---|---|---|
| Appearance | | ⁺PASS | ⁺PASS | ⁺PASS | ⁺PASS | ⁺PASS | ⁺PASS |
| | | | No change | No change | No change | No change | No change |
| | | | in clarity | in clarity | in clarity | in clarity | in clarity |
| | | | or turbidity | or turbidity | or turbidity | or turbidity | or turbidity |
| pH | | 5.7 | 5.8 | 5.8 | 6.1 | 5.8 | 5.7 |
| Total Protein by Solo-VPE (mg/mL) | | 212 | 210 | 212 | 212 | 204 | 213 |
| Turbidity (increase in OD at 405 nm) | | 0.00 | 0.01 | 0.01 | 0.02 | 0.03 | 0.06 |
| % Relative Potency by Bioassay | | 118 | N/A | N/A | N/A | N/A | 88 |
| Purity | % HMW | 0.9 | 1.3 | 1.4 | 1.9 | 3.8 | 5.8 |
| by | % Main | 98.9 | 98.4 | 98.3 | 97.6 | 95.5 | 93.4 |
| SE-UPLC | % LMW | 0.2 | 0.3 | 0.4 | 0.5 | 0.7 | 0.8 |
| Purity by | % Purity | 93.1 | 92.5 | 92.8 | 91.9 | 88.5 | 87.2 |
| Reduced | % LMW | 0.7 | 0.7 | 0.6 | 1.1 | 3.9 | 5.2 |
| MCE-SDS | % NGHC | 5.2 | 5.6 | 5.4 | 5.8 | 5.8 | 5.8 |
| Purity by | % Purity | 98.1 | 98.0 | 97.6 | 97.3 | 94.7 | 91.5 |
| Non-Reduced | % LMW | 2.0 | 2.0 | 2.4 | 2.7 | 4.2 | 7.0 |
| MCE-SDS | % HMW | 0.0 | 0.0 | 0.0 | 0.0 | 1.2 | 1.5 |
| Charge Variant | % Region 1 | 28.8 | 28.5 | 32.7 | 35.1 | 41.3 | 44.6 |
| Analysis | % Region 2 | 60.2 | 57.1 | 53.5 | 47.6 | 39.8 | 35.7 |
| by iCIEF | % Region 3 | 11.0 | 14.4 | 13.9 | 17.3 | 18.9 | 19.7 |
| Particulate Matter | 2-10 μm | 6908 | N/A | N/A | N/A | 4667 | 8617 |
| (Micro-Flow Imaging; | ≥10 μm | 50 | N/A | N/A | N/A | 35 | 336 |
| particles/mL) | ≥25 μm | 6 | N/A | N/A | N/A | 9 | 36 |

^{~}USP Type 1 clear glass, 2 mL vial with 13 mm gray chlorobutyl rubber stopper with FluroTec stored upright and containing 200 mg/mL H4H12166P, 20 mM histidine, pH 5.8, 100 mM L-arginine hydrochloride, 2% (w/v) sucrose, 0.15% (w/v) polysorbate 80.
⁺Pass = Clear to pale yellow, and essentially free from visible particulates.

A comparison of the results between the 2 mL and 5 mL glass vials revealed that factors such as container size (2 mL and 5 mL) and fill volume (0.5 mL and 2.5 mL) did not have a meaningful impact on the degradation pathways or trends in key quality attributes in the H4H12166P formulation.

### Example 6: Agitation and Freeze/Thaw Testing for 200 mg/mL H4H12166P

Stability studies evaluated the stability of a liquid, aqueous H4H12166P 200 mg/mL formulation under agitation and freeze/thaw conditions.

TABLE 6-1
Summary of Stress/Incubation Conditions

| Storage/Stress | Incubation/ | |
|---|---|---|
| Condition | Stress Time | Conditions |
| Agitation (250 rpm | 0, 24 and 48 hours | Room temperature |
| on orbital shaker) | | (~22° C.) |
| Freeze/thaw | 0, 2 and 4 cycles | Each cycle included −30° C. |
| | | freeze for at least 2 hours |
| | | and room temperature thaw |

### Results

The attributes of an aqueous formulation comprising 200 mg/mL H4H12166P, 20 mM histidine, pH 5.8, 100 mM L-arginine hydrochloride, 2% (w/v) sucrose, 0.15% (w/v) polysorbate 80, following agitation or freeze/thaw in 6R Type 1 borosilicate glass vials (stored upright), are set forth in Table 6-2 and 6-3.

TABLE 6-2
Stability Study with Agitation.

| | | Agitation | | |
|---|---|---|---|---|
| Assay | | t = 0 | 24 hours | 48 hours |
| Color and Appearance* | | ⁺PASS | ⁺PASS | ⁺PASS |
| Turbidity (Increase in OD at 405 nm) | | 0.00 | 0.00 | 0.00 |
| pH | | 5.8 | 5.8 | 5.8 |
| % Total Protein Recovered by RP-HPLC | | 100 | 99 | 100 |
| Purity by SE-UPLC | % HMW | 1.1 | 1.1 | 1.1 |
| | % Main | 98.5 | 98.5 | 98.5 |
| | % LMW | 0.4 | 0.4 | 0.4 |
| Purity by Reduced | % Heavy + | 95.3 | 95.7 | 95.7 |
| MCE-SDS | Light chain | | | |
| | % NGHC | 2.6 | 2.4 | 2.1 |
| | % LMW | 0.8 | 1.2 | 1.1 |
| Purity by Non- | % Main peak | 96.7 | 96.4 | 96.6 |
| Reduced MCE-SDS | % LMW | 3.2 | 3.3 | 3.2 |
| | % HMW | 0.1 | 0.3 | 0.2 |
| Charge Variant | % Acidic | 29.5 | 28.9 | 29.0 |
| Analysis by iCIEF | % Main | 56.9 | 57.9 | 57.3 |
| | % Basic | 13.6 | 13.3 | 13.7 |
| Charge Variant | % Acidic | 22.9 | 22.8 | 22.8 |
| Analysis by CEX | % Main | 59.9 | 60.2 | 60.1 |
| | % Basic | 17.2 | 17.0 | 17.2 |
| Particulate Analysis | ≥10 μm | 0 | NR | 3 |
| by MFI (particles/mL) | ≥25 μm | 0 | NR | 0 |

⁺Pass = Clear to pale yellow, and essentially free from visible particulates.

TABLE 6-3
Stability Studies with Freeze-Thaw

| | | Freeze/Thaw | | |
|---|---|---|---|---|
| Assay | | t = 0 | 2 cycles | 4 cycles |
| Color and Appearance* | | ⁺PASS | ⁺PASS | ⁺PASS |
| Turbidity (Increase in OD at 405 nm) | | 0.00 | 0.00 | 0.01 |
| pH | | 5.8 | 5.8 | 5.8 |
| % Total Protein Recovered by RP-HPLC | | 100 | 100 | 99 |
| Purity by SE-UPLC | % HMW | 1.1 | 1.1 | 1.1 |
| | % Main | 98.4 | 98.4 | 98.4 |
| | % LMW | 0.5 | 0.5 | 0.5 |
| Charge Variant | % Acidic | 28.6 | 26.3 | 26.3 |
| Analysis by iCIEF | % Main | 58.2 | 59.8 | 59.6 |
| | % Basic | 13.3 | 13.9 | 14.1 |
| Charge Variant | % Acidic | 22.9 | 23.0 | 23.1 |
| Analysis by CEX | % Main | 60.1 | 60.8 | 60.5 |
| | % Basic | 17.0 | 16.3 | 16.4 |
| Particulate Analysis | ≥10 μm | 3 | NR | 3 |
| by MFI (particles/mL) | ≥25 μm | 0 | NR | 2 |

⁺Pass = Clear to pale yellow, and essentially free from visible particulates.

No appreciable change in physical or chemical stability after 48 hr of agitation and of 4 cycles of freeze/thaw for aqueous formulation comprising of 200 mg/mL H4H12166P.

### Example 7: Long-term Stability Testing for 274 mg/mL H4H12166P

Stability studies evaluated the stability of a liquid, aqueous H4H12166P 274 mg/mL formulation under long term storage (5° C.).

### Results

The attributes of an aqueous formulation comprising 274 mg/mL H4H12166P, 20 mM histidine, pH 5.8, 100 mM L-arginine hydrochloride, 2% (w/v) sucrose, 0.15% (w/v) polysorbate 80, following 15.5 months of long term (5° C.) storage 5 mL container, are set forth in Table 7-1. Table 7-2 summarizes the attributes of liquid, aqueous H4H12166P 274 mg/mL formulation after freeze/thaw.

TABLE 7-1
Stability after Agitation

| | | | 2 | 7 | 6 | 15.5 |
|---|---|---|---|---|---|---|
| Assay | | t = 0 | days | days | months | months |
| Color and Appearance* | | ⁺PASS | ⁺PASS | ⁺PASS | ⁺PASS | ⁺PASS |
| pH | | 5.9 | 5.9 | 5.9 | 5.9 | 5.9 |
| Total Protein by RP-UPLC (mg/mL) | | 282 | 282 | 282 | 294 | 281 |
| Turbidity (increase in OD at 405 nm) | | — | 0.02 | 0.03 | 0.01 | 0.02 |
| Purity | % HMW | 2.1 | 2.1 | 2.1 | 2.3 | 2.4 |
| by | % Main | 97.5 | 97.5 | 97.5 | 97.1 | 96.9 |
| SE-UPLC | % LMW | 0.4 | 0.4 | 0.4 | 0.6 | 0.7 |
| Charge Variant | % Acidic | 29.2 | 29.2 | 29.2 | 28.0 | 28.3 |
| Analysis by | % Main Peak | 58.0 | 58.2 | 58.2 | 58.7 | 58.4 |
| CEX-UPLC | % Basic | 12.8 | 12.7 | 12.6 | 13.3 | 13.3 |
| Particulate Matter | 2-10 μm | 129 | 109 | 579 | 61 | 94 |
| (Micro-Flow Imaging, | ≥10 μm | 14 | 26 | 32 | 35 | 18 |
| Particles/mL) | ≥25 μm | 8 | 3 | 6 | 11 | 3 |

*Pass = Clear to pale yellow, and essentially free from visible particulates.

TABLE 7-2
Stability after Freeze-Thaw

| Assay | | t = 0 | 2 cycles | 4 cycles | 8 cycles |
|---|---|---|---|---|---|
| Color and Appearance | | ⁺PASS | ⁺PASS | ⁺PASS | ⁺PASS |
| pH | | 5.9 | 5.9 | 5.9 | 5.8 |
| Total Protein by RP-UPLC (mg/mL) | | 282 | 281 | 284 | 282 |
| Turbidity (increase in OD at 405 nm) | | — | 0.02 | 0.02 | 0.03 |
| Purity by SE-UPLC | % HMW | 2.1 | 2.1 | 2.2 | 2.2 |
| | % Main | 97.5 | 97.5 | 97.4 | 97.4 |
| | % LMW | 0.4 | 0.4 | 0.4 | 0.4 |
| Charge Variant | % Acidic | 29.2 | 29.2 | 29.2 | 29.3 |
| Analysis by | % Main Peak | 58.0 | 58.0 | 58.3 | 57.7 |
| CEX-UPLC | % Basic | 12.8 | 12.7 | 12.6 | 13.0 |
| | 2-10 μm | 129 | 111 | 233 | 216 |
| Particulate Matter | ≥10 μm | 14 | 24 | 35 | 21 |
| (Micro-Flow Imaging, | ≥25 μm | 8 | 3 | 14 | 6 |
| Particiles/mL) | | | | | |

*Pass = Clear to pale yellow, and essentially free from visible particulates.

No appreciable change in physical or chemical stability after 15.5 months at 5° C. of 8 cycles of freeze/thaw for aqueous formulation comprising of 274 mg/mL H4H12166P.

### Example 8: Comparison of Viscosities of H4H12166P

Viscosity was measured using a Rheosense Initium automatic viscometer. Prior to analysis, standards and formulations were filtered through a 0.22 μm PVDF spin filter. Two standards of known viscosities were measured before the unknown sample viscosity analysis: 2 cP and glycerol standards. Viscosity measurements were performed at different temperatures (5-40° C.) of aqueous formulations comprising of 161-274 mg/mL H4H12166P, 20 mM histidine, pH 5.8, 100 mM L-arginine hydrochloride, 2% (w/v) sucrose, 0.15% (w/v) polysorbate 80. Table 8-1 summarizes measured viscosity.

TABLE 8-1
Viscosity of H4H12166P at various temperatures^{#}

| Temper- | Viscosity (cP) | | | | | | | |
|---|---|---|---|---|---|---|---|---|
| ature | H4H12166P Concentration (mg/mL) | | | | | | | |
| (° C.) | 161 | 177 | 190 | 198 | 205 | 221 | 240 | 274 |
| 5 | 12.7 | 19.5 | 25.0 | 28.1 | 36.9 | 47.6 | 83.5 | 114.6 |
| 10 | 10.1 | 14.9 | 18.8 | 21.2 | 27.5 | 34.4 | 57.9 | 82.2 |
| 15 | 8.2 | 11.8 | 14.8 | 16.5 | 21.1 | 26.3 | 42.9 | 62.2 |
| 20 | 6.8 | 9.6 | 11.9 | 13.2 | 16.7 | 20.6 | 33.0 | 48.4 |
| 25 | 5.7 | 8.0 | 9.8 | 10.8 | 13.5 | 16.6 | 26.0 | 38.0 |
| 30 | 4.9 | 6.8 | 8.1 | 9.0 | 11.2 | 13.6 | 21.1 | Not |
| | | | | | | | | measured |
| 35 | 4.2 | 5.7 | 7.0 | 7.6 | 9.4 | 11.3 | 17.1 | Not |
| | | | | | | | | measured |
| 40 | 3.7 | 5.0 | 6.0 | 6.5 | 7.9 | 9.5 | 14.3 | 30.8 |

^{#}Containing 20 mM histidine, pH 5.8, 100 mM L-arginine hydrochloride, 2% (w/v) sucrose, 0.15% (w/v) polysorbate 80.

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants, to relate to each and every individual publication, database entry (e.g., Genbank sequences or GeneID entries), patent application, or patent even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

1. An aqueous pharmaceutical formulation comprising about 161-274 mg/ml or more antigen-binding protein that binds specifically to complement factor 5 (anti-C5), and a pharmaceutically acceptable carrier; which is characterized by one or more selected from:
(i) a viscosity of about 8, about 9, about 10, about 11, about 12, about 13, about 14, about 14.3, about 15, less than about 15, about 16, about 17, about 18, about 19, about 20 or from about 8 to about 20 cP, at about 20° C.;
(ii) a viscosity of about 50 cP at about 20° C.;
(iii) an osmolality of about 267-404 mmol/kg;
(iv) exhibits an increase of about 0.1 or about 0.2% or less in high molecular weight (HMW) species after orbital shaker agitation for about 6 hours at 250 rpm;
(v) exhibits an increase of about 0.5, about 0.6 or about 0.7% in high molecular weight (HMW) species after orbital shaker agitation for about 24 hours at 250 rpm;
(vi) exhibits an increase of about 0.8, about 0.9 or about 1.0% in high molecular weight (HMW) species after 4 freeze-thaw cycles, wherein freezing is at about −30° C. and thawing is at about 22° C.;
(vii) exhibits an increase of about 1.0, about 1.1, about 1.2 or about 1.3% in high molecular weight (HMW) species after 8 freeze-thaw cycles, wherein freezing is at about −30° C. and thawing is at about 22° C.;
(viii) the anti-C5 antigen-binding protein has Tₘ1 (onset) of about 58.0° C., a Tₘ1 of about 61.7° C.; and a Tₘ2 of about 73.2° C., as measured using differential scanning calorimetry (DSC);
(ix) one or more methionines in the anti-C5 antigen-binding protein are oxidized;
(x) comprises less than or equal to about 0.1 EU/mg endotoxin content;
(xi) comprises about 0.9% high molecular weight (HMW) species, as measured by size-exclusion ultra-high performance liquid chromatography (SE-UPLC);
(xii) comprises about 0.9, about 1.0, about 1.1, about 1.2 or about 0.9-1.2% high molecular weight (HMW) species, as measured by size-exclusion ultra-high performance liquid chromatography (SE-UPLC), after about 0, about 1, about 3, about 6, about 9 or about 12 months of storage at about 5° C.;
(xiii) comprises about 0.9, about 1.0, about 1.1, about 1.2, about 1.3, about 1.4, about 1.5, or about 1.1-1.5% high molecular weight (HMW) species, as measured by size-exclusion ultra-high performance liquid chromatography (SE-UPLC), after about 0, about 0.5, about 1, about 3 or about 6 months of storage at about 25° C.;
(xiv) comprises about 0.9, about 1.0, about 1.1, about 1.2, about 1.3, about 1.4, about 1.9, about 3.8, about 5.8% or about 1.3-5.8% high molecular weight (HMW) species, as measured by size-exclusion ultra-high performance liquid chromatography (SE-UPLC), after about 0, about 0.25, about 0.5, about 1, about 2 or about 3 months of storage at about 40° C.;
(xv) comprises about 0.2, about 0.3, about 0.4, about 0.5 about 0.6 or about 0.2-0.6% low molecular weight (LMW) species, as measured by size-exclusion ultra-high performance liquid chromatography (SE-UPLC), after about 0, about 1, about 3, about 6, about 9 or about 12 months of storage at about 5° C.;
(xvi) comprises about 0.2, about 0.3, about 0.4 or about 0.3-0.4% low molecular weight (LMW) species, as measured by size-exclusion ultra-high performance liquid chromatography (SE-UPLC), after about 0, about 0.5, about 1, about 3 or about 6 months of storage at about 25° C.;
(xvii) comprises about 0.3, about 0.4, about 0.5, about 0.6, about 0.7, about 0.8 or about 0.3-0.8% low molecular weight (LMW) species, as measured by size-exclusion ultra-high performance liquid chromatography (SE-UPLC), after about 0, about 0.25, about 0.5, about 1, about 2 or about 3 months of storage at about 40° C.;
(xviii) comprises about 98, about 98.3, about 98.4, about 98.5, about 98.6, about 98.7, about 98.8, about 99 or about 98.3-98.8% main species, as measured by size-exclusion ultra-high performance liquid chromatography (SE-UPLC), after about 0, about 1, about 3 or about 6, about 9 or about 12 months of storage at about 5° C.;
(xix) comprises about 98, about 98.1, about 98.2, about 98.3, about 98.4, about 98.5, about 98.6, about 98.7, about 98.8, about 98.9, about 99 or about 98.1-98.6% main species, as measured by size-exclusion ultra-high performance liquid chromatography (SE-UPLC), after about 0, about 0.5, about 1, about 3 or about 6 months of storage at about 25° C.;
(xx) comprises about 98.9, about 98.4 about 98.3, about 97.6, about 95.5, about 93.4 or about 93.4-98.4% main species, as measured by size-exclusion ultra-high performance liquid chromatography (SE-UPLC), after about 0, about 0.25, about 0.5, about 1, about 2 or about 6 months of storage at about 40° C.;
(xxi) comprises about 29% acidic charge variants, about 11% basic charge variants and/or about 60% main species, as measured by imaging capillary isoelectric focusing (iCIEF);
(xxii) comprises about 30% acidic charge variants, about 14% basic charge variants and/or about 56% main species, e.g., as measured by imaging capillary isoelectric focusing (iCIEF), e.g., after about 6 months of storage at about 5° C.;
(xxiii) comprises about 33% acidic charge variants, about 20% basic charge variants and/or about 47% main species, as measured by imaging capillary isoelectric focusing (iCIEF), after about 6 months of storage at about 25° C.;
(xxiv) comprises about 45% acidic charge variants, about 36% basic charge variants and/or about 20% main species, as measured by imaging capillary isoelectric focusing (iCIEF), after about 3 months of storage at about 40° C.; and
(xxv) comprises a buffer; L-arginine; water; and, optionally, an oligosaccharide; and
optionally, a non-ionic detergent, with a pH of up to about and a viscosity of about 6.8 to 48.4 cP at 20° C.

2. The pharmaceutical formulation of claim 1, wherein the formulation comprises:
a buffer;
L-arginine;
water;
and,
optionally, an oligosaccharide; and
optionally, a non-ionic detergent,
with a pH of up to about 5.8, 6.1 or 5.5-6.1; and a viscosity, at 20° C., of about 6.8, about 9.6, about 11.9, about 13.2, about 14 cP, about 14.3 cP, about 15 cP, about 16.7, about 20.6, about 33.0, about 48.4, or about 13.2-16.7.

3. The pharmaceutical formulation of claim 1, wherein the antigen-binding protein is an antibody or antigen-binding fragment thereof.

4. The pharmaceutical formulation of claim 1, wherein the concentration of antigen-binding protein that binds specifically to C5 is about 274 mg/ml or about 200 mg/ml.

5. The pharmaceutical formulation of claim 1, wherein the L-arginine is L-arginine-hydrochloride.

6. The pharmaceutical formulation of claim 1, wherein the concentration of arginine is about 100 mM.

7. The pharmaceutical formulation of claim 1, comprising an oligosaccharide, wherein the oligosaccharide is sucrose, mannitol, dextrose, glycerol, TMAO (trimethylamine N-oxide), trehalose, ethylene glycol, glycine betaine, xylitol or sorbitol.

8. The pharmaceutical formulation of claim 1, comprising an oligosaccharide, wherein the oligosaccharide is sucrose.

9. The pharmaceutical formulation of claim 1, comprising an oligosaccharide, wherein the concentration of oligosaccharide is about 2% (w/v).

10. The pharmaceutical formulation of claim 1, wherein the pH is about 5.8.

11. The pharmaceutical formulation of claim 1, wherein the antigen-binding protein that binds specifically to C5 is: H2M11683N; H2M11686N; H4H12159P; H4H12161P; H4H12163P; H4H12164P; H4H12166P; H4H12166P2; H4H12166P3; H4H12166P4; H4H12166P5; H4H12166P6; H4H12166P7; H4H12166P8; H4H12166P9; H4H12166P10; H4H12167P; H4HI2168P; H4HI2169P; H4H12170P; H4H12171P; H4H12175P; H4H12176P2; H4H12177P2; H4H12183P2; H2M11682N; H2M11684N; H2M11694N; H2M11695N, ravulizumab, eculizumab, crovalimab, tesidolumab, mubodina, IFX-1 and/or olendalizumab.

12. The pharmaceutical formulation of claim 1, wherein the antigen-binding protein that binds specifically to C5 is pozelimab.

13. The pharmaceutical formulation of claim 1, wherein the buffer is phosphate buffer, acetate buffer, citrate buffer, histidine buffer, or imidazole buffer.

14. The pharmaceutical formulation of claim 1, wherein the buffer is histidine buffer.

15. The pharmaceutical formulation of claim 1, wherein the concentration of buffer is about 20 mM.

16. The pharmaceutical formulation of claim 1, comprising a non-ionic detergent, wherein the non-ionic detergent is polyoxyethylene-based detergent or glycosidic compound-based detergent, polysorbate-20, polysorbate-80 or tween-20.

17. The pharmaceutical formulation of claim 1, comprising a non-ionic detergent, wherein the non-ionic detergent is polysorbate-80.

18. The pharmaceutical formulation of claim 1, comprising a non-ionic detergent, wherein the concentration of non-ionic detergent is about 0.15% (w/v).

19. The pharmaceutical formulation of claim 1, comprising:
about 180-210 mg/ml antigen-binding protein that binds specifically to C5;
about 20±4 mM buffer;
about 100±20 mM L-arginine;
about 2±0.4% (w/v) oligosaccharide;
about 0.15±0.075% (w/v) non-ionic detergent; and
water,
pH 5.8±0.3.

20. The pharmaceutical formulation of claim 1, comprising:
about 200 mg/ml antigen-binding protein that binds specifically to C5;
about 20±4 mM histidine buffer;
about 100±20 mM L-arginine;
about 2±0.4% (w/v) sucrose;
about 0.15±0.075% (w/v) polysorbate-80; and
water,
pH 5.8±0.3.

21. An aqueous pharmaceutical formulation comprising:
about 200 mg/ml H2M11683N; about 20 mM histidine buffer; about 100 mM L-arginine; about 2% (w/v) sucrose; about 0.15% (w/v) polysorbate-80 (PS-80) and water, pH about 5.8;
about 200 mg/ml H2M11686N; about 20 mM histidine buffer; about 100 mM L-arginine; about 2% (w/v) sucrose; about 0.15% (w/v) polysorbate-80 and water, pH about 5.8;
about 200 mg/ml H4H12159P; about 20 mM histidine buffer; about 100 mM L-arginine; about 2% (w/v) sucrose; about 0.15% (w/v) polysorbate-80 and water, pH about 5.8;
about 200 mg/ml H4H12161P; about 20 mM histidine buffer; about 100 mM L-arginine; about 2% (w/v) sucrose; about 0.15% (w/v) polysorbate-80 and water, pH about 5.8;
about 200 mg/ml H4H12163P; about 20 mM histidine buffer; about 100 mM L-arginine; about 2% (w/v) sucrose; about 0.15% (w/v) polysorbate-80 and water, pH about 5.8;
about 200 mg/ml H4H12164P; about 20 mM histidine buffer; about 100 mM L-arginine; about 2% (w/v) sucrose; about 0.15% (w/v) polysorbate-80 and water, pH about 5.8;
about 200 mg/ml H4H12166P; about 20 mM histidine buffer; about 100 mM L-arginine; about 2% (w/v) sucrose; about 0.15% (w/v) polysorbate-80 and water, pH about 5.8;
about 200 mg/ml H4H12166P2; about 20 mM histidine buffer; about 100 mM L-arginine; about 2% (w/v) sucrose; about 0.15% (w/v) polysorbate-80 and water, pH about 5.8;
about 200 mg/ml H4H12166P3; about 20 mM histidine buffer; about 100 mM L-arginine; about 2% (w/v) sucrose; about 0.15% (w/v) polysorbate-80 and water, pH about 5.8;
about 200 mg/ml H4H12166P4; about 20 mM histidine buffer; about 100 mM L-arginine; about 2% (w/v) sucrose; about 0.15% (w/v) polysorbate-80 and water, pH about 5.8;
about 200 mg/ml H4H12166P5; about 20 mM histidine buffer; about 100 mM L-arginine; about 2% (w/v) sucrose; about 0.15% (w/v) polysorbate-80 and water, pH about 5.8;
about 200 mg/ml H4H12166P6; about 20 mM histidine buffer; about 100 mM L-arginine; about 2% (w/v) sucrose; about 0.15% (w/v) polysorbate-80 and water, pH about 5.8;
about 200 mg/ml H4H12166P7; about 20 mM histidine buffer; about 100 mM L-arginine; about 2% (w/v) sucrose; about 0.15% (w/v) polysorbate-80 and water, pH about 5.8;
about 200 mg/ml H4H12166P8; about 20 mM histidine buffer; about 100 mM L-arginine; about 2% (w/v) sucrose; about 0.15% (w/v) polysorbate-80 and water, pH about 5.8;
about 200 mg/ml H4H12166P9; about 20 mM histidine buffer; about 100 mM L-arginine; about 2% (w/v) sucrose; about 0.15% (w/v) polysorbate-80 and water, pH about 5.8;
about 200 mg/ml H4H12167P; about 20 mM histidine buffer; about 100 mM L-arginine; about 2% (w/v) sucrose; about 0.15% (w/v) polysorbate-80 and water, pH about 5.8;
about 200 mg/ml H4H12168P; about 20 mM histidine buffer; about 100 mM L-arginine; about 2% (w/v) sucrose; about 0.15% (w/v) polysorbate-80 and water, pH about 5.8;
about 200 mg/ml H4H12169P; about 20 mM histidine buffer; about 100 mM L-arginine; about 2% (w/v) sucrose; about 0.15% (w/v) polysorbate-80 and water, pH about 5.8;
about 200 mg/ml H4H12170P; about 20 mM histidine buffer; about 100 mM L-arginine; about 2% (w/v) sucrose; about 0.15% (w/v) polysorbate-80 and water, pH about 5.8;
about 200 mg/ml H4H12171P; about 20 mM histidine buffer; about 100 mM L-arginine; about 2% (w/v) sucrose; about 0.15% (w/v) polysorbate-80 and water, pH about 5.8;
about 200 mg/ml H4H12175P; about 20 mM histidine buffer; about 100 mM L-arginine; about 2% (w/v) sucrose; about 0.15% (w/v) polysorbate-80 and water, pH about 5.8;
about 200 mg/ml H4H12176P2; about 20 mM histidine buffer; about 100 mM L-arginine; about 2% (w/v) sucrose; about 0.15% (w/v) polysorbate-80 and water, pH about 5.8;
about 200 mg/ml H4H12177P2; about 20 mM histidine buffer; about 100 mM L-arginine; about 2% (w/v) sucrose; about 0.15% (w/v) polysorbate-80 and water, pH about 5.8;
about 200 mg/ml H4H12183P2; about 20 mM histidine buffer; about 100 mM L-arginine; about 2% (w/v) sucrose; about 0.15% (w/v) polysorbate-80 and water, pH about 5.8;
about 200 mg/ml H2M11682N; about 20 mM histidine buffer; about 100 mM L-arginine; about 2% (w/v) sucrose; about 0.15% (w/v) polysorbate-80 and water, pH about 5.8;
about 200 mg/ml H2M11684N; about 20 mM histidine buffer; about 100 mM L-arginine; about 2% (w/v) sucrose; about 0.15% (w/v) polysorbate-80 and water, pH about 5.8;
about 200 mg/ml H2M11694N; about 20 mM histidine buffer; about 100 mM L-arginine; about 2% (w/v) sucrose; about 0.15% (w/v) polysorbate-80 and water, pH about 5.8;
about 200 mg/ml H2M11695N; about 20 mM histidine buffer; about 100 mM L-arginine; about 2% (w/v) sucrose; about 0.15% (w/v) polysorbate-80 and water, pH about 5.8;
about 200 mg/ml H4H12166P, about 5 mM histidine, about 2.5% (w/v) proline, about 5% (w/v) sucrose, about 150 mM L-arginine-HCl, about 0.2% (w/v) PS-80, and water, pH about 5.7;
about 135 mg/ml H4H12166P, about 20 mM histidine, about 5% (w/v) proline, about 10% (w/v) sucrose, about 150 mM L-arginine-HCl, about 0.02% (w/v) PS-80, and water, pH about 5.7;
about 160 mg/ml H4H12166P, about 5 mM histidine, about 5% (w/v) sucrose, about 75 mM L-arginine-HCl, about 0.02% (w/v) PS-80, and water, pH about 6.2;
about 120 mg/ml H4H12166P, about 40 mM histidine, about 5% (w/v) proline, about 0.02% (w/v) PS-80, and water, pH about 6.8;
about 120 mg/ml H4H12166P, about 40 mM histidine, about 0.02% (w/v) PS-80, and water, pH about 5.7;
about 160 mg/ml H4H12166P, about 5 mM histidine, about 2.5% (w/v) proline, about 10% (w/v) sucrose, about 0.2% (w/v) PS-80, and water, pH about 6.8;
about 120 mg/ml H4H12166P, about 40 mM histidine, about 5% (w/v) proline, about 150 mM L-arginine-HCl, about 0.02% (w/v) PS-80, and water, pH about 5.7;
about 200 mg/ml H4H12166P, about 5 mM histidine, about 0.2% (w/v) PS-80, and water, pH about 5.7;
about 200 mg/ml H4H12166P, about 20 mM histidine, about 5% (w/v) proline, about 10% (w/v) sucrose, about 0.2% (w/v) PS-80, and water, pH about 6.2;
about 120 mg/ml H4H12166P, about 40 mM histidine, about 150 mM L-arginine-HCl, about 0.02% (w/v) PS-80, and water, pH about 6.8;
about 200 mg/ml H4H12166P, about 5 mM histidine, about 5% (w/v) proline, about 150 mM L-arginine-HCl, about 0.2% (w/v) PS-80, and water, pH about 6.2;
about 120 mg/ml H4H12166P, about 20 mM histidine, about 5% (w/v) proline, about 5% (w/v) sucrose, about 150 mM L-arginine-HCl, about 0.2% (w/v) PS-80, and water, pH about 6.8;
about 120 mg/ml H4H12166P, about 5 mM histidine, about 10% (w/v) sucrose, about 0.2% (w/v) PS-80, and water, pH about 5.7;
about 120 mg/ml H4H12166P, about 5 mM histidine, about 0.2% (w/v) PS-80, and water, pH about 6.8;
about 120 mg/ml H4H12166P, about 5 mM histidine, about 150 mM L-arginine-HCl, about 0.2% (w/v) PS-80, and water, pH about 5.7;
about 175 mg/ml H4H12166P, about 40 mM histidine, about 5% (w/v) proline, about 0.2% (w/v) PS-80, and water, pH about 5.7;
about 200 mg/ml H4H12166P, about 5 mM histidine, about 10% (w/v) sucrose, about 150 mM L-arginine-HCl, about 0.2% (w/v) PS-80, and water, pH about 6.8;
about 185 mg/ml H4H12166P, about 40 mM histidine, about 10% (w/v) sucrose, about 0.02% (w/v) PS-80, and water, pH about 5.7;
about 120 mg/ml H4H12166P, about 40 mM histidine, about 10% (w/v) sucrose, about 150 mM L-arginine-HCl, about 0.2% (w/v) PS-80, and water, pH about 5.7;
about 120 mg/ml H4H12166P, about 40 mM histidine, about 10% (w/v) sucrose, about 0.02% (w/v) PS-80, and water, pH about 6.8;
about 120 mg/ml H4H12166P, about 5 mM histidine, about 5% (w/v) proline, about 0.02% (w/v) PS-80, and water, pH about 5.7;
about 170 mg/ml H4H12166P, about 40 mM histidine, about 5% (w/v) proline, about 10% (w/v) sucrose, about 150 mM L-arginine-HCl, about 0.02% (w/v) PS-80, and water, pH about 6.8;
about 120 mg/ml H4H12166P, about 40 mM histidine, about 5% (w/v) proline, about 10% (w/v) sucrose, about 0.02% (w/v) PS-80, and water, pH about 5.7;
about 120 mg/ml H4H12166P, about 5 mM histidine, about 2.5% (w/v) proline, about 10% (w/v) sucrose, about 150 mM L-arginine-HCl, about 0.02% (w/v) PS-80, and water, pH about 6.2;
about 200 mg/ml H4H12166P, about 5 mM histidine, about 5% (w/v) proline, about 10% (w/v) sucrose, about 75 mM L-arginine-HCl, about 0.2% (w/v) PS-80, and water, pH about 5.7;
about 120 mg/ml H4H12166P, about 5 mM histidine, about 5% (w/v) proline, about 10% (w/v) sucrose, about 75 mM L-arginine-HCl, about 0.2% (w/v) PS-80, and water, pH about 6.8;
about 160 mg/ml H4H12166P, about 5 mM histidine, about 5% (w/v) proline, about 150 mM L-arginine-HCl, about 0.2% (w/v) PS-80, and water, pH about 6.8;
about 200 mg/ml H4H12166P, about 20 mM histidine, about 2.5% (w/v) proline, about 75 mM L-arginine-HCl, about 0.02% (w/v) PS-80, and water, pH about 6.8;
about 170 mg/ml H4H12166P, about 35 mM histidine, about 150 mM L-arginine-HCl, about 0.02% (w/v) PS-80, and water, pH about 5.7;
about 183 mg/ml H4H12166P, about 40 mM histidine, about 0.2% (w/v) PS-80, and water, pH about 6.8;
about 200 mg/ml H4H12166P, about 5 mM histidine, about 5% (w/v) proline, about 5% (w/v) sucrose, about 0.02% (w/v) PS-80, and water, pH about 6.8.
about 160 mg/ml H4H12166P, about 40 mM histidine, about 2.5% (w/v) proline, about 5% (w/v) sucrose, about 75 mM L-arginine-HCl, about 0.2% (w/v) PS-80, and water, pH about 6.2;
about 187 mg/ml H4H12166P, about 40 mM histidine, about 0.02% (w/v) PS-80, and water, pH about 5.7;
about 200 mg/ml±20 mg/ml H4H12166P, about 10-24 mM histidine, pH about 5.5±0.6, water and about 100 mM±20 mM L-arginine;
about 200 mg/ml H4H12166P, about 20 mM histidine, pH about 5.8, water, about 2% sucrose, about 100 mM L-arginine, and about 0.15% polysorbate-80;
about 274 mg/ml H4H12166P, about 20 mM histidine, pH about 5.8, water, about 2% sucrose, about 100 mM L-arginine, and about 0.15% polysorbate-80; or
about 180-210 mg/ml H4H12166P, about 16-24 mM histidine, pH about 5.5-6.1, water, about 1.6-2.4% (w/v) sucrose, about 80-120 mM L-arginine, and about 0.075-0.225% (w/v) polysorbate-80.

22. The pharmaceutical formulation of claim 1 in association with a further therapeutic agent.

23. The pharmaceutical formulation of claim 22, wherein the further therapeutic agent is an oligonucleotide, anti-coagulant, warfarin, aspirin, heparin, phenindione, fondaparinux, idraparinux, a thrombin inhibitor, argatroban, lepirudin, bivalirudin, dabigatran, an anti-inflammatory drug, a corticosteroid, a non-steroidal anti-inflammatory drug (NSAID), an antihypertensive, an angiotensin-converting enzyme inhibitor, an immunosuppressive agent, vincristine, cyclosporine A, or methotrexate, a fibrinolytic agent ancrod, E-aminocaproic acid, antiplasmin-a1, prostacyclin, defibrotide, a lipid-lowering agent, an inhibitor of hydroxymethylglutaryl CoA reductase, an anti-CD20 agent, rituximab, an anti-TNFalpha agent, infliximab, an anti-seizure agent, magnesium sulfate, a C3 inhibitor and/or an anti-thrombotic agent.

24. The pharmaceutical formulation of claim 22, wherein the further therapeutic agent is an oligonucleotide which is:
a DNA oligonucleotide,
an RNA oligonucleotide,
a single stranded DNA oligonucleotide,
a single stranded RNA oligonucleotide,
a double stranded DNA oligonucleotide, or
a double stranded RNA oligonucleotide;
optionally, wherein the oligonucleotide is conjugated to a sugar.

25. A method for making the pharmaceutical formulation of claim 1, comprising admixing the antigen-binding protein and the carrier.

26. A pharmaceutical formulation which is a product of the method of claim 25.

27. An intravenous formulation comprising a pharmaceutical formulation of claim 1 in an aqueous intravenous solution.

28. The intravenous formulation of claim 27, wherein the aqueous intravenous solution has a volume of about 250 ml, 500 ml, 750 ml or 1000 ml.

29. The intravenous formulation of claim 1, comprising one or more selected from the group consisting of NaCl, dextrose, potassium salt, potassium chloride, calcium salt, calcium chloride, sodium lactate and lactate salt.

30. The intravenous formulation of claim 27, wherein the aqueous intravenous solution is 0.9% Normal Saline, Lactated Ringers, 5% dextrose in water; 0.45% Normal Saline; 0.33% NaCl; 0.225% NaCl; 2.5% dextrose in water; 3% NaCl; 5% NaCl; 5% dextrose in 0.45% NaCl; 5% dextrose and 0.45% NaCl; 5% dextrose in 0.9% NaCl; 5% dextrose in Lactated Ringer's; Lactated Ringers that contains 0.6% NaCl; 10% dextrose in water; 20% dextrose in water; or 50% dextrose in water.

31. The intravenous formulation of claim 27, which contains an amount of said pharmaceutical formulation such that, when administered to a subject, a dose of 1, 3, 10, 15 or 30 mg/kg body weight is achieved.

32. An intravenous glass or plastic bag or glass or plastic bottle comprising the intravenous formulation of claim 27.

33. The intravenous formulation, bag or bottle of one of claim 27, which is sterile.

34. A method for making the intravenous formulation of claim 27, comprising introducing said pharmaceutical formulation into the aqueous intravenous solution.

35. An intravenous formulation which is a product of the method of claim 34.

36. A vessel or injection device comprising the formulation of claim 1.

37. A method for reducing the viscosity of a composition comprising about 161-274 mg/ml pozelimab comprising combining said pozelimab with arginine.

38. The method of claim 37, wherein the final concentration of arginine in the composition is about 50 mM or about 100 mM.

39. The method of claim 37, wherein the final concentration of pozelimab is about 150 mg/ml, about 161 mg/ml, 175 mg/ml, about 177 mg/ml, about 190 mg/ml, about 198 mg/ml, 200 mg/ml, 205 mg/ml, 211 mg/ml, 220 mg/ml, 221 mg/ml, 240 mg/ml, 242 mg/ml or 274 mg/ml, at least about 150 mg/ml, at least about 175 mg/ml, at least about 200 mg/ml, at least about 211 mg/ml, at least about 220 mg/ml, at least about 242 mg/ml, or at least about 274 mg/ml.

40. The method of claim 37, wherein viscosity is cP as measured at 20° C.

41. The method of claim 37, wherein viscosity is reduced by about 30% or about 30-42%.

42. A method for administering a pharmaceutical formulation or intravenous formulation of one of claim 1 to a subject comprising introducing the pharmaceutical formulation or intravenous formulation and, optionally, a further therapeutic agent, into the body of the subject.

43. The method of claim 42, wherein the pharmaceutical formulation or intravenous formulation is introduced into the body of the subject separately from the further therapeutic agent.

44. The method of claim 42, wherein the pharmaceutical formulation and/or intravenous formulation; and the further therapeutic agent is administered parenterally.

45. The method of claim 42, wherein parenterally is intravenously, intramuscularly, or subcutaneously.

46. A method for treating or preventing a C5-associated disease in a subject in need thereof comprising administering a therapeutically effective amount of the pharmaceutical formulation and/or intravenous formulation of claim 1 and, optionally, a further therapeutic agent, to the subject.

47. The method of claim 46, wherein said pharmaceutical formulation and/or intravenous formulation is administered separately from the further therapeutic agent.

48. The method of claim 46, wherein the C5-associated disease is selected from the group consisting of: atypical hemolytic uremic syndrome (aHUS), paroxysmal nocturnal hemoglobinuria (PNH), myasthenia gravis or CHAPLE disease.

49. The method of claim 46, wherein the C5-associated disease is selected from the group consisting of adult respiratory distress syndrome; age-related macular degeneration (AMD); allergy; Alport's syndrome; Alzheimer's disease; antiphospholipid syndrome (APS); asthma; atherosclerosis; atypical hemolytic uremic syndrome (aHUS); autoimmune disease; autoimmune hemolytic anemia (AIHA); balloon angioplasty; bronchoconstriction; bullous pemphigoid; burns; C3 glomerulopathy; capillary leak syndrome; cardiovascular disorder; catastrophic antiphospholipid syndrome (CAPS); cerebrovascular disorder; CHAPLE disease; chemical injury; chronic obstructive pulmonary disease (COPD); cold agglutinin disease (CAD); corneal and/or retinal tissue; Crohn's disease; Degos disease; dense deposit disease (DDD); dermatomyositis; diabetes; diabetic angiopathy; diabetic macular edema (DME); diabetic nephropathy; diabetic retinopathy; dilated cardiomyopathy; disorder of inappropriate or undesirable complement activation; dyspnea; emphysema; epidermolysis bullosa; epilepsy; fibrogenic dust disease; frostbite; geographic atrophy (GA); glomerulonephritis; glomerulopathy; Goodpasture's Syndrome; Graves' disease; Guillain Barre Syndrome; Hashimoto's thyroiditis; hemodialysis complications; hemolysis-elevated liver enzymes-and low platelets (HELLP) syndrome; hemolytic anemia; hemoptysis; Henoch-Schonlein purpura nephritis; hereditary angioedema; hyperacute allograft rejection; hypersensitivity pneumonitis; idiopathic thrombocytopenic purpura (ITP); IgA nephropathy; immune complex disorder; immune complex vasculitis; immune complex-associated inflammation; infectious disease; inflammation caused by an autoimmune disease; inflammatory disorder; inherited CD59 deficiency; injury due to inert dusts and/or minerals; interleukin-2 induced toxicity during IL-2 therapy; ischemia-reperfusion injury; Kawasaki's disease; lung disease or disorder; lupus nephritis; membrane proliferative glomerulonephritis; membrano-proliferative nephritis; mesenteric artery reperfusion after aortic reconstruction; mesenteric/enteric vascular disorder; multifocal motor neuropathy (MMN); multiple sclerosis; myasthenia gravis; myocardial infarction; myocarditis; neurological disorder; neuromyelitis optica; obesity; ocular angiogenesis; ocular neovascularization affecting choroidal; organic dust disease; parasitic disease; Parkinson's disease; paroxysmal nocturnal hemoglobinuria (PNH); Pauci-immune vasculitis; pemphigus; percutaneous transluminal coronary angioplasty (PTCA); peripheral vascular disorder; pneumonia; post-ischemic reperfusion condition; post-pump syndrome in cardiopulmonary bypass; post-pump syndrome in renal bypass; progressive kidney failure; proliferative nephritis; proteinuric kidney disease; psoriasis; pulmonary embolism; pulmonary fibrosis; pulmonary infarction; pulmonary vasculitis; recurrent fetal loss; renal disorder; renal ischemia; renal ischemia-reperfusion injury; renovascular disorder; restenosis following stent placement; rheumatoid arthritis; rotational atherectomy; schizophrenia; sepsis; septic shock; SLE nephritis; smoke injury; spinal cord injury; spontaneous fetal loss; stroke; systemic inflammatory response to sepsis; systemic lupus erythematosus (SLE); systemic lupus erythematosus-associated vasculitis; Takayasu's disease; thermal injury; thrombotic thrombocytopenic purpura (TTP); traumatic brain injury; type I diabetes; typical hemolytic uremic syndrome; uveitis; vasculitis; vasculitis associated with rheumatoid arthritis; venous gas embolus (VGE); and xenograft rejection.

50. A method for reducing complement activity in the body of a subject in need thereof comprising administering a therapeutically effective amount of pharmaceutical formulation and/or intravenous formulation of claim 1; and, optionally, a further therapeutic agent, to the subject.

51. The method of claim 50, wherein the pharmaceutical formulation and/or intravenous formulation is administered separately from the further therapeutic agent.

52. A method for switching therapeutic regimens for treating or preventing a C5-associated disease comprising ceasing administering a first therapeutic agent and then administering a therapeutically effective amount of pharmaceutical formulation and/or intravenous formulation of claim 1 and, optionally, a further therapeutic agent, to the subject, wherein said pharmaceutical formulation and/or intravenous formulation comprises an anti-C5 antigen-binding protein which is different from that of the first therapeutic agent.

53. The method of claim 52, wherein the pharmaceutical formulation and/or intravenous formulation is administered separately from the further therapeutic agent.

54. The method of claim 52, wherein the first therapeutic agent is tesidolumab, crovalimab, eculizumab, or ravulizumab.


SEQUENCE LISTING (369 sequences)

SEQ ID NO: 1 - DNA, 357 bp, Artificial Sequence
  synthetic
caggtgcagctggtggagtctgggggaggcgtggtccagcctggaaggtccctgagactctcctgtgtagcgtctggattcaccttcagtagttatggcattcactgggtccgccaggctccaggcaaggggctggagtgggtggcagttatatgggatgatggaaataatataaactattcagactccgtgaagggccgattcatcatctccagagacaattccaggaagacagtgtatctgcaaatgaacagcctgagaggcgaggacacggctgtttattactgtgcgagagatgcccccatagcaccagtccctgactattggggccagggaaccctggtcaccgtctcctca

SEQ ID NO: 2 - protein, 119 aa, Artificial Sequence
  synthetic
QVQLVESGGGVVQPGRSLRLSCVASGFTFSSYGIHWVRQAPGKGLEWVAVIWDDGNNINYSDSVKGRFIISRDNSRKTVYLQMNSLRGEDTAVYYCARDAPIAPVPDYWGQGTLVTVSS

SEQ ID NO: 3 - DNA, 24 bp, Artificial Sequence
  synthetic
ggattcaccttcagtagttatggc

SEQ ID NO: 4 - protein, 8 aa, Artificial Sequence
  synthetic
GFTFSSYG

SEQ ID NO: 5 - DNA, 24 bp, Artificial Sequence
  synthetic
atatgggatgatggaaataatata

SEQ ID NO: 6 - protein, 8 aa, Artificial Sequence
  synthetic
IWDDGNNI

SEQ ID NO: 7 - DNA, 36 bp, Artificial Sequence
  synthetic
gcgagagatgcccccatagcaccagtccctgactat

SEQ ID NO: 8 - protein, 12 aa, Artificial Sequence
  synthetic
ARDAPIAPVPDY

SEQ ID NO: 9 - DNA, 321 bp, Artificial Sequence
  synthetic
gacatccagatgacccagtctccttccaccctgtctgcatctgtaggagacagagtcaccatcacttgccgggccagtcagagtattagtagttggttggcctggtatcagcagaaaccagggaaagcccctaagctcctgatctataaggcgtctagtttagacactggggtcccatcaaggttcagcggcagtggatctgggacagagttcactctcaccatcagcagcctgcagcctgatgattttgcaacttattactgccaacagtataatacttattcgtacacttttggcctggggaccaaactggagatcaaa

SEQ ID NO: 10 - protein, 107 aa, Artificial Sequence
  synthetic
DIQMTQSPSTLSASVGDRVTITCRASQSISSWLAWYQQKPGKAPKLLIYKASSLDTGVPSRFSGSGSGTEFTLTISSLQPDDFATYYCQQYNTYSYTFGLGTKLEIK

SEQ ID NO: 11 - DNA, 18 bp, Artificial Sequence
  synthetic
cagagtattagtagttgg

SEQ ID NO: 12 - protein, 6 aa, Artificial Sequence
  synthetic
QSISSW

SEQ ID NO: 13 - DNA, 9 bp, Artificial Sequence
  synthetic
aaggcgtct

SEQ ID NO: 14 - protein, 3 aa, Artificial Sequence
  synthetic
KAS

SEQ ID NO: 15 - DNA, 27 bp, Artificial Sequence
  synthetic
caacagtataatacttattcgtacact

SEQ ID NO: 16 - protein, 9 aa, Artificial Sequence
  synthetic
QQYNTYSYT

SEQ ID NO: 17 - DNA, 357 bp, Artificial Sequence
  synthetic
caggtgcaactggtggagtctgggggaggcttggtcaagcctggagggtccctgagactctcctgtgcagcttctggattcaccttcagtgactactacatgagctggatccgccaggctccagggaaggggctggagtgggtttcatatattagcagtagtggtaataccataaaatatgcagactctatgaagggccgattcaccatctccagggacaacgccaagaaatcactgtttgtggaaatgaacagcctgagagccgaggacacggccgtgtattactgtgcgaggtataaaagttcgtccgactactttgaccactggggccagggaaccctggtcaccgtctcctca

SEQ ID NO: 18 - protein, 119 aa, Artificial Sequence
  synthetic
QVQLVESGGGLVKPGGSLRLSCAASGFTFSDYYMSWIRQAPGKGLEWVSYISSSGNTIKYADSMKGRFTISRDNAKKSLFVEMNSLRAEDTAVYYCARYKSSSDYFDHWGQGTLVTVSS

SEQ ID NO: 19 - DNA, 24 bp, Artificial Sequence
  synthetic
ggattcaccttcagtgactactac

SEQ ID NO: 20 - protein, 8 aa, Artificial Sequence
  synthetic
GFTFSDYY

SEQ ID NO: 21 - DNA, 24 bp, Artificial Sequence
  synthetic
attagcagtagtggtaataccata

SEQ ID NO: 22 - protein, 8 aa, Artificial Sequence
  synthetic
ISSSGNTI

SEQ ID NO: 23 - DNA, 36 bp, Artificial Sequence
  synthetic
gcgaggtataaaagttcgtccgactactttgaccac

SEQ ID NO: 24 - protein, 12 aa, Artificial Sequence
  synthetic
ARYKSSSDYFDH

SEQ ID NO: 25 - DNA, 321 bp, Artificial Sequence
  synthetic
gaaattgtgttgacacagtctccagccaccctgtctttgtctccaggggaaagagccaccctctcctgcagggccagtcagagtgttaggagttacttagcctggtaccaacagaaacctggccaggctcccaggctcctcatctatgatgcatccaacagggccactgccatcccagccaggttcagtggcagtgggtctgggacagacttcactctcaccatcagcagcctagagcctgaagatttagcagtttattactgtcagcagtctggcaactggccgctcactttcggcggagggaccaaggtggagatcaaa

SEQ ID NO: 26 - protein, 107 aa, Artificial Sequence
  synthetic
EIVLTQSPATLSLSPGERATLSCRASQSVRSYLAWYQQKPGQAPRLLIYDASNRATAIPARFSGSGSGTDFTLTISSLEPEDLAVYYCQQSGNWPLTFGGGTKVEIK

SEQ ID NO: 27 - DNA, 18 bp, Artificial Sequence
  synthetic
cagagtgttaggagttac

SEQ ID NO: 28 - protein, 6 aa, Artificial Sequence
  synthetic
QSVRSY

SEQ ID NO: 29 - DNA, 9 bp, Artificial Sequence
  synthetic
gatgcatcc

SEQ ID NO: 30 - protein, 3 aa, Artificial Sequence
  synthetic
DAS

SEQ ID NO: 31 - DNA, 27 bp, Artificial Sequence
  synthetic
cagcagtctggcaactggccgctcact

SEQ ID NO: 32 - protein, 9 aa, Artificial Sequence
  synthetic
QQSGNWPLT

SEQ ID NO: 33 - DNA, 357 bp, Artificial Sequence
  synthetic
caggtgcagctggtggagtctgggggaggcgtggtccagcctgggaggtccttgagactctcctgtggagcgtctggattcaccttcagtacttatggcatgcactgggtccgccaggctccaggcaaggggctggagtgggtggcagttatctgggatgatggaaataataaatattatgcagactccgtgaagggccgattcaccatctccagagacaattcgaagaacacgctgtatctgcagatgaacagcctgagagccgaggacacggctgtttattactgtgcgagagattcagaggtcgccccagttggggactactggggccagggcaccctggtcaccgtctcctca

SEQ ID NO: 34 - protein, 119 aa, Artificial Sequence
  synthetic
QVQLVESGGGVVQPGRSLRLSCGASGFTFSTYGMHWVRQAPGKGLEWVAVIWDDGNNKYYADSVKGRFTISRDNSKNTLYLQMNSLRAEDTAVYYCARDSEVAPVGDYWGQGTLVTVSS

SEQ ID NO: 35 - DNA, 24 bp, Artificial Sequence
  synthetic
ggattcaccttcagtacttatggc

SEQ ID NO: 36 - protein, 8 aa, Artificial Sequence
  synthetic
GFTFSTYG

SEQ ID NO: 37 - DNA, 24 bp, Artificial Sequence
  synthetic
atctgggatgatggaaataataaa

SEQ ID NO: 38 - protein, 8 aa, Artificial Sequence
  synthetic
IWDDGNNK

SEQ ID NO: 39 - DNA, 36 bp, Artificial Sequence
  synthetic
gcgagagattcagaggtcgccccagttggggactac

SEQ ID NO: 40 - protein, 12 aa, Artificial Sequence
  synthetic
ARDSEVAPVGDY

SEQ ID NO: 41 - DNA, 321 bp, Artificial Sequence
  synthetic
gacatccagatgacccagtctccttccaccctgtctgcatctgtaggagacagagtcactatcatttgccgggccagtcagagtattaacaggtggttggcctggtatcagcagaaaccagggaaggcccctaaactcctgatctataaggcgtctagtttagaaagtggggtcccatcaaggttcagcggcagtggatctgggacagaattcactctcaccatcagcagcctgcagcctgatgattttgcagcttattactgccaacagtataatgattattcgtacacttttggccaggggaccaagctggagatcaaa

SEQ ID NO: 42 - protein, 107 aa, Artificial Sequence
  synthetic
DIQMTQSPSTLSASVGDRVTIICRASQSINRWLAWYQQKPGKAPKLLIYKASSLESGVPSRFSGSGSGTEFTLTISSLQPDDFAAYYCQQYNDYSYTFGQGTKLEIK

SEQ ID NO: 43 - DNA, 18 bp, Artificial Sequence
  synthetic
cagagtattaacaggtgg

SEQ ID NO: 44 - protein, 6 aa, Artificial Sequence
  synthetic
QSINRW

SEQ ID NO: 45 - DNA, 9 bp, Artificial Sequence
  synthetic
aaggcgtct

SEQ ID NO: 46 - protein, 3 aa, Artificial Sequence
  synthetic
KAS

SEQ ID NO: 47 - DNA, 27 bp, Artificial Sequence
  synthetic
caacagtataatgattattcgtacact

SEQ ID NO: 48 - protein, 9 aa, Artificial Sequence
  synthetic
QQYNDYSYT

SEQ ID NO: 49 - DNA, 366 bp, Artificial Sequence
  synthetic
gaggtgcagctggtggagtctgggggagacttggtccagcctggagggtccctgagactctcctgtgcagcctctggattcaccttcagtgaccactatatggactgggtccgccaggctccagggaaggggctggactggattggccgtattagaaacaaagctaacgcttataacacagaatacgccgcgtctgtgagaggcagattcaccatctcaagagatgattcacagaatttactgtatctgcaaatgaacagcctgaaaaccgatgacacggccgtatattattgtgttagagtctggaactacgcctacttcgctatggacgtctggggccaagggaccacggtcaccgtctcctca

SEQ ID NO: 50 - protein, 122 aa, Artificial Sequence
  synthetic
EVQLVESGGDLVQPGGSLRLSCAASGFTFSDHYMDWVRQAPGKGLDWIGRIRNKANAYNTEYAASVRGRFTISRDDSQNLLYLQMNSLKTDDTAVYYCVRVWNYAYFAMDVWGQGTTVTVSS

SEQ ID NO: 51 - DNA, 24 bp, Artificial Sequence
  synthetic
ggattcaccttcagtgaccactat

SEQ ID NO: 52 - protein, 8 aa, Artificial Sequence
  synthetic
GFTFSDHY

SEQ ID NO: 53 - DNA, 30 bp, Artificial Sequence
  synthetic
attagaaacaaagctaacgcttataacaca

SEQ ID NO: 54 - protein, 10 aa, Artificial Sequence
  synthetic
IRNKANAYNT

SEQ ID NO: 55 - DNA, 39 bp, Artificial Sequence
  synthetic
gttagagtctggaactacgcctacttcgctatggacgtc

SEQ ID NO: 56 - protein, 13 aa, Artificial Sequence
  synthetic
VRVWNYAYFAMDV

SEQ ID NO: 57 - DNA, 321 bp, Artificial Sequence
  synthetic
gacatccagatgacccagtctccatcctccctatctgcatctgtgggagacagagtcaccatcacttgccggtcaagtcagaacattggaatctttttaaactggtatcaacaaaaaccaggggaagcccctaacctcctgatctccgctgcatccagtttacacagtggggtcccttcaaggttcagtggcagtgggtctgggacagatttcactctcaccatcggcagtctgcagcctgaagattttgcgacttactactgtcaacagacgtacaataccatattcactttcggccctgggaccaaagtggatatcaaa

SEQ ID NO: 58 - protein, 107 aa, Artificial Sequence
  synthetic
DIQMTQSPSSLSASVGDRVTITCRSSQNIGIFLNWYQQKPGEAPNLLISAASSLHSGVPSRFSGSGSGTDFTLTIGSLQPEDFATYYCQQTYNTIFTFGPGTKVDIK

SEQ ID NO: 59 - DNA, 18 bp, Artificial Sequence
  synthetic
cagaacattggaatcttt

SEQ ID NO: 60 - protein, 6 aa, Artificial Sequence
  synthetic
QNIGIF

SEQ ID NO: 61 - DNA, 9 bp, Artificial Sequence
  synthetic
gctgcatcc

SEQ ID NO: 62 - protein, 3 aa, Artificial Sequence
  synthetic
AAS

SEQ ID NO: 63 - DNA, 27 bp, Artificial Sequence
  synthetic
caacagacgtacaataccatattcact

SEQ ID NO: 64 - protein, 9 aa, Artificial Sequence
  synthetic
QQTYNTIFT

SEQ ID NO: 65 - DNA, 363 bp, Artificial Sequence
  synthetic
gaggtgcagctggtggagtctgggggagacttggtacagcctggggggtccctgagactctcctgtgcagcctctggattcacctttagcagctatgccatgaactgggtccgccagggtccagggaagggactggagtgggtctcagctattagtggtcgtggtgatagtacatactacgcagactccgtgaagggccggctcaccatctccagagacaattccaagaacacgctgtatctgcaaatgaacagcctgagagccgaggacacggccgtatattactgtgtgaaagagggggagcaactcgtctactggtacttcgatctctggggccgtggcaccctggtcaccgtctcctca

SEQ ID NO: 66 - protein, 121 aa, Artificial Sequence
  synthetic
EVQLVESGGDLVQPGGSLRLSCAASGFTFSSYAMNWVRQGPGKGLEWVSAISGRGDSTYYADSVKGRLTISRDNSKNTLYLQMNSLRAEDTAVYYCVKEGEQLVYWYFDLWGRGTLVTVSS

SEQ ID NO: 67 - DNA, 24 bp, Artificial Sequence
  synthetic
ggattcacctttagcagctatgcc

SEQ ID NO: 68 - protein, 8 aa, Artificial Sequence
  synthetic
GFTFSSYA

SEQ ID NO: 69 - DNA, 24 bp, Artificial Sequence
  synthetic
attagtggtcgtggtgatagtaca

SEQ ID NO: 70 - protein, 8 aa, Artificial Sequence
  synthetic
ISGRGDST

SEQ ID NO: 71 - DNA, 42 bp, Artificial Sequence
  synthetic
gtgaaagagggggagcaactcgtctactggtacttcgatctc

SEQ ID NO: 72 - protein, 14 aa, Artificial Sequence
  synthetic
VKEGEQLVYWYFDL

SEQ ID NO: 73 - DNA, 321 bp, Artificial Sequence
  synthetic
gacatccagatgacccagtctccatcctccctgtctgcatctgtaggagacagagtcaccatcacttgccgggcaagtcagaccattagcaactttttacattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagatttttcaacttacttctgtcaacagagttacactaccccgctcactttcggcggagggaccaaggtggagatcaaa

SEQ ID NO: 74 - protein, 107 aa, Artificial Sequence
  synthetic
DIQMTQSPSSLSASVGDRVTITCRASQTISNFLHWYQQKPGKAPKLLIYAASSLQSGVPSRFSGSGSGTDFTLTISSLQPEDFSTYFCQQSYTTPLTFGGGTKVEIK

SEQ ID NO: 75 - DNA, 18 bp, Artificial Sequence
  synthetic
cagaccattagcaacttt

SEQ ID NO: 76 - protein, 6 aa, Artificial Sequence
  synthetic
QTISNF

SEQ ID NO: 77 - DNA, 9 bp, Artificial Sequence
  synthetic
gctgcatcc

SEQ ID NO: 78 - protein, 3 aa, Artificial Sequence
  synthetic
AAS

SEQ ID NO: 79 - DNA, 27 bp, Artificial Sequence
  synthetic
caacagagttacactaccccgctcact

SEQ ID NO: 80 - protein, 9 aa, Artificial Sequence
  synthetic
QQSYTTPLT

SEQ ID NO: 81 - DNA, 363 bp, Artificial Sequence
  synthetic
gaggtgcagctggtggagtctgggggaggcttggtgaggtcgggggggtccctgagactctcctgtgcagcctctggattcacctttaacagatatgccatgacctgggtccgccaggctccagggaaggggctggagtgggtctcagctataagtggtagtggtagcagcacatactacacagactccgtgaaaggccggttcaccatctccagagacaattccaagaattcggtggatctgcaaatgcacagcctgagagtcgaagacacggccatatattattgtgcgagagggactacagtcactacggggtacggtatggacgtctggggccaagggaccacggtcaccgtctcctca

SEQ ID NO: 82 - protein, 121 aa, Artificial Sequence
  synthetic
EVQLVESGGGLVRSGGSLRLSCAASGFTFNRYAMTWVRQAPGKGLEWVSAISGSGSSTYYTDSVKGRFTISRDNSKNSVDLQMHSLRVEDTAIYYCARGTTVTTGYGMDVWGQGTTVTVSS

SEQ ID NO: 83 - DNA, 24 bp, Artificial Sequence
  synthetic
ggattcacctttaacagatatgcc

SEQ ID NO: 84 - protein, 8 aa, Artificial Sequence
  synthetic
GFTFNRYA

SEQ ID NO: 85 - DNA, 24 bp, Artificial Sequence
  synthetic
ataagtggtagtggtagcagcaca

SEQ ID NO: 86 - protein, 8 aa, Artificial Sequence
  synthetic
ISGSGSST

SEQ ID NO: 87 - DNA, 42 bp, Artificial Sequence
  synthetic
gcgagagggactacagtcactacggggtacggtatggacgtc

SEQ ID NO: 88 - protein, 14 aa, Artificial Sequence
  synthetic
ARGTTVTTGYGMDV

SEQ ID NO: 89 - DNA, 321 bp, Artificial Sequence
  synthetic
gacatccagatgacccagtctccatcctccctgtctgcatctgtaggagacagagtcaccttcacttgccaggcgagtcaggacattaccaattctttaaattggtatcaacagaaacctgggagagcccctaagctcctgatctacgatgcatcgtatttgaaggcaggggtcccatcaagattcagtggaagtggatctgggacagattttactttcaccatcagcagcctgcagcctgaagatattgcaacatattactgtcaacaatatgatgatctcccatacacttttggccaggggaccaagctggagatcaaa

SEQ ID NO: 90 - protein, 107 aa, Artificial Sequence
  synthetic
DIQMTQSPSSLSASVGDRVTFTCQASQDITNSLNWYQQKPGRAPKLLIYDASYLKAGVPSRFSGSGSGTDFTFTISSLQPEDIATYYCQQYDDLPYTFGQGTKLEIK

SEQ ID NO: 91 - DNA, 18 bp, Artificial Sequence
  synthetic
caggacattaccaattct

SEQ ID NO: 92 - protein, 6 aa, Artificial Sequence
  synthetic
QDITNS

SEQ ID NO: 93 - DNA, 9 bp, Artificial Sequence
  synthetic
gatgcatcg

SEQ ID NO: 94 - protein, 3 aa, Artificial Sequence
  synthetic
DAS

SEQ ID NO: 95 - DNA, 27 bp, Artificial Sequence
  synthetic
caacaatatgatgatctcccatacact

SEQ ID NO: 96 - protein, 9 aa, Artificial Sequence
  synthetic
QQYDDLPYT

SEQ ID NO: 97 - DNA, 360 bp, Artificial Sequence
  synthetic
caggtgcagctgcaggagtcgggcccaggactggtgaagccttcggagaccctgtccctcacctgcactgtctctggtgactccgtcagtagttcctactggacctggatccggcagcccccagggaagggactggagtggattggctatatctattacagtgggagttccaactacaacccctccctcaagagtcgagccaccatttcagtagacacgtccaagaaccagttctccctgaagctgagttctgtgaccgctgcggacacggccgtatattactgtgcgagagaagggaacgtggatacaactatgatatttgactactggggccagggaaccctggtcaccgtctcctca

SEQ ID NO: 98 - protein, 120 aa, Artificial Sequence
  synthetic
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSS

SEQ ID NO: 99 - DNA, 24 bp, Artificial Sequence
  synthetic
ggtgactccgtcagtagttcctac

SEQ ID NO: 100 - protein, 8 aa, Artificial Sequence
  synthetic
GDSVSSSY

SEQ ID NO: 101 - DNA, 21 bp, Artificial Sequence
  synthetic
atctattacagtgggagttcc

SEQ ID NO: 102 - protein, 7 aa, Artificial Sequence
  synthetic
IYYSGSS

SEQ ID NO: 103 - DNA, 42 bp, Artificial Sequence
  synthetic
gcgagagaagggaacgtggatacaactatgatatttgactac

SEQ ID NO: 104 - protein, 14 aa, Artificial Sequence
  synthetic
AREGNVDTTMIFDY

SEQ ID NO: 105 - DNA, 321 bp, Artificial Sequence
  synthetic
gccatccagatgacccagtctccatcctccctgtctgcatctgtaggagacagagtcaccatcacttgccgggcaagtcagggcattagaaatgatttaggctggtatcaacagaaaccagggaaagcccctaaactcctgatctatgctgcatccagtttacaaagtggggtcccatcgaggttcgccggccgtggatctggcacagatttcactctcaccatcagcagcctgcagcctgaagattttgcaacttattactgtctacaagatttcaattacccgtggacgttcggccaagggaccaaggtggaaatcaaa

SEQ ID NO: 106 - protein, 107 aa, Artificial Sequence
  synthetic
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIK

SEQ ID NO: 107 - DNA, 18 bp, Artificial Sequence
  synthetic
cagggcattagaaatgat

SEQ ID NO: 108 - protein, 6 aa, Artificial Sequence
  synthetic
QGIRND

SEQ ID NO: 109 - DNA, 9 bp, Artificial Sequence
  synthetic
gctgcatcc

SEQ ID NO: 110 - protein, 3 aa, Artificial Sequence
  synthetic
AAS

SEQ ID NO: 111 - DNA, 27 bp, Artificial Sequence
  synthetic
ctacaagatttcaattacccgtggacg

SEQ ID NO: 112 - protein, 9 aa, Artificial Sequence
  synthetic
LQDFNYPWT

SEQ ID NO: 113 - DNA, 321 bp, Artificial Sequence
  synthetic
gccatccagatgacccagtctccatcctccctgtctgcatctgtaggagacagagtcaccatcacttgccgggcaagtcagggcattagaaatgatttaggctggtatcaacagaaaccagggaaagcccctaaactcctgatctatgctgcatccagtttacaaagtggggtcccatcgaggttcgccggccgtggatctggcacagatttcactctcaccatcagcagcctgcagcctgaagattttgcaacttattactgtcatcaagatttcaattacccgtggacgttcggccaagggaccaaggtggaaatcaaa

SEQ ID NO: 114 - protein, 107 aa, Artificial Sequence
  synthetic
AIQMTQSPSSLSASVGDRVTITCRASQGIRNDLGWYQQKPGKAPKLLIYAASSLQSGVPSRFAGRGSGTDFTLTISSLQPEDFATYYCHQDFNYPWTFGQGTKVEIK

SEQ ID NO: 115 - DNA, 18 bp, Artificial Sequence
  synthetic
cagggcattagaaatgat

SEQ ID NO: 116 - protein, 6 aa, Artificial Sequence
  synthetic
QGIRND

SEQ ID NO: 117 - DNA, 9 bp, Artificial Sequence
  synthetic
gctgcatcc

SEQ ID NO: 118 - protein, 3 aa, Artificial Sequence
  synthetic
AAS

SEQ ID NO: 119 - DNA, 27 bp, Artificial Sequence
  synthetic
catcaagatttcaattacccgtggacg

SEQ ID NO: 120 - protein, 9 aa, Artificial Sequence
  synthetic
HQDFNYPWT

SEQ ID NO: 121 - DNA, 360 bp, Artificial Sequence
  synthetic
caggtgcagctgcaggagtcgggcccaggactggtgaagccttcggagaccctgtccctcacctgcactgtctctggtgactccgtcagtagttcctactggacctggatccggcagcccccagggaagggactggagtggattggctatatctattacagtgggagttccaactacaacccctccctcaagagtcgagccaccatttcagtagacacgtccaagaaccagttctccctgaagctgagttctgtgaccgctgcggacacggccgtatattactgtgcgagagaacataacgtggatacaactatgatatttgactactggggccagggaaccctggtcaccgtctcctca

SEQ ID NO: 122 - protein, 120 aa, Artificial Sequence
  synthetic
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREHNVDTTMIFDYWGQGTLVTVSS

SEQ ID NO: 123 - DNA, 24 bp, Artificial Sequence
  synthetic
ggtgactccgtcagtagttcctac

SEQ ID NO: 124 - protein, 8 aa, Artificial Sequence
  synthetic
GDSVSSSY

SEQ ID NO: 125 - DNA, 21 bp, Artificial Sequence
  synthetic
atctattacagtgggagttcc

SEQ ID NO: 126 - protein, 7 aa, Artificial Sequence
  synthetic
IYYSGSS

SEQ ID NO: 127 - DNA, 42 bp, Artificial Sequence
  synthetic
gcgagagaacataacgtggatacaactatgatatttgactac

SEQ ID NO: 128 - protein, 14 aa, Artificial Sequence
  synthetic
AREHNVDTTMIFDY

SEQ ID NO: 129 - DNA, 321 bp, Artificial Sequence
  synthetic
gccatccagatgacccagtctccatcctccctgtctgcatctgtaggagacagagtcaccatcacttgccgggcaagtcagggcattagaaatgatttaggctggtatcaacagaaaccagggaaagcccctaaactcctgatctatgctgcatccagtttacaaagtggggtcccatcgaggttcgccggccgtggatctggcacagatttcactctcaccatcagcagcctgcagcctgaagattttgcaacttattactgtctacaagatttcaattacccgtggcacttcggccaagggaccaaggtggaaatcaaa

SEQ ID NO: 130 - protein, 107 aa, Artificial Sequence
  synthetic
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWHFGQGTKVEIK

SEQ ID NO: 131 - DNA, 18 bp, Artificial Sequence
  synthetic
cagggcattagaaatgat

SEQ ID NO: 132 - protein, 6 aa, Artificial Sequence
  synthetic
QGIRND

SEQ ID NO: 133 - DNA, 9 bp, Artificial Sequence
  synthetic
gctgcatcc

SEQ ID NO: 134 - protein, 3 aa, Artificial Sequence
  synthetic
AAS

SEQ ID NO: 135 - DNA, 27 bp, Artificial Sequence
  synthetic
ctacaagatttcaattacccgtggcac

SEQ ID NO: 136 - protein, 9 aa, Artificial Sequence
  synthetic
LQDFNYPWH

SEQ ID NO: 137 - DNA, 360 bp, Artificial Sequence
  synthetic
caggtgcagctgcaggagtcgggcccaggactggtgaagccttcggagaccctgtccctcacctgcactgtctctggtgactccgtcagtagttcctactggacctggatccggcagcccccagggaagggactggagtggattggctatatctattacagtgggagttccaactacaacccctccctcaagagtcgagccaccatttcagtagacacgtccaagaaccagttctccctgaagctgagttctgtgaccgctgcggacacggccgtatattactgtgcgagagaagggaacgtggatacaactatgatacatgactactggggccagggaaccctggtcaccgtctcctca

SEQ ID NO: 138 - protein, 120 aa, Artificial Sequence
  synthetic
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIHDYWGQGTLVTVSS

SEQ ID NO: 139 - DNA, 24 bp, Artificial Sequence
  synthetic
ggtgactccgtcagtagttcctac

SEQ ID NO: 140 - protein, 8 aa, Artificial Sequence
  synthetic
GDSVSSSY

SEQ ID NO: 141 - DNA, 21 bp, Artificial Sequence
  synthetic
atctattacagtgggagttcc

SEQ ID NO: 142 - protein, 7 aa, Artificial Sequence
  synthetic
IYYSGSS

SEQ ID NO: 143 - DNA, 42 bp, Artificial Sequence
  synthetic
gcgagagaagggaacgtggatacaactatgatacatgactac

SEQ ID NO: 144 - protein, 14 aa, Artificial Sequence
  synthetic
AREGNVDTTMIHDY

SEQ ID NO: 145 - DNA, 360 bp, Artificial Sequence
  synthetic
caggtgcagctgcaggagtcgggcccaggactggtgaagccttcggagaccctgtccctcacctgcactgtctctggtgactccgtcagtagttcctactggacctggatccggcagcccccagggaagggactggagtggattggctatatctattacagtgggagttccaactacaacccctccctcaagagtcgagccaccatttcagtagacacgtccaagaaccagttctccctgaagctgagttctgtgaccgctgcggacacggccgtatattactgtgcgagagaagggaacgtggatcacactatgatatttgactactggggccagggaaccctggtcaccgtctcctca

SEQ ID NO: 146 - protein, 120 aa, Artificial Sequence
  synthetic
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDHTMIFDYWGQGTLVTVSS

SEQ ID NO: 147 - DNA, 24 bp, Artificial Sequence
  synthetic
ggtgactccgtcagtagttcctac

SEQ ID NO: 148 - protein, 8 aa, Artificial Sequence
  synthetic
GDSVSSSY

SEQ ID NO: 149 - DNA, 21 bp, Artificial Sequence
  synthetic
atctattacagtgggagttcc

SEQ ID NO: 150 - protein, 7 aa, Artificial Sequence
  synthetic
IYYSGSS

SEQ ID NO: 151 - DNA, 42 bp, Artificial Sequence
  synthetic
gcgagagaagggaacgtggatcacactatgatatttgactac

SEQ ID NO: 152 - protein, 14 aa, Artificial Sequence
  synthetic
AREGNVDHTMIFDY

SEQ ID NO: 153 - DNA, 360 bp, Artificial Sequence
  synthetic
caggtgcagctggtggagtctgggggaggcttggtcaagcctggagggtccctgagactctcctgtgcagcctctggattcaccttcagtgactcctacatgtcctggatccgtcaggctccagggaagggactagagtggatttcatacattggtagtagtggtaataccttttactacgcagactctgtgaagggccggttcaccatttccagagacaacgccaacaatttactgtatctgcaaatgaccagcctgagagccgaggacacggccgtgtattactgtgcgagagaagaaggcgatttttggagtgccgttgactcctggggccagggaaccctggtcaccgtctcctca

SEQ ID NO: 154 - protein, 120 aa, Artificial Sequence
  synthetic
QVQLVESGGGLVKPGGSLRLSCAASGFTFSDSYMSWIRQAPGKGLEWISYIGSSGNTFYYADSVKGRFTISRDNANNLLYLQMTSLRAEDTAVYYCAREEGDFWSAVDSWGQGTLVTVSS

SEQ ID NO: 155 - DNA, 24 bp, Artificial Sequence
  synthetic
ggattcaccttcagtgactcctac

SEQ ID NO: 156 - protein, 8 aa, Artificial Sequence
  synthetic
GFTFSDSY

SEQ ID NO: 157 - DNA, 24 bp, Artificial Sequence
  synthetic
attggtagtagtggtaataccttt

SEQ ID NO: 158 - protein, 8 aa, Artificial Sequence
  synthetic
IGSSGNTF

SEQ ID NO: 159 - DNA, 39 bp, Artificial Sequence
  synthetic
gcgagagaagaaggcgatttttggagtgccgttgactcc

SEQ ID NO: 160 - protein, 13 aa, Artificial Sequence
  synthetic
AREEGDFWSAVDS

SEQ ID NO: 161 - DNA, 321 bp, Artificial Sequence
  synthetic
gacatccagttgacccagtctccatccttcctgtctgcatctgtaggagacagagtcaccatcacttgctgggccagtcagggcattagcagttatttagcctggtatcagcaaaaaccaggtaaagcccctaaactcctgatccatactgcatccactttgcaaagtggggtcccatcaaggttcagcggcagtggatctgggacagaattcactctcacaatcagcaacctgcagcctgaagattttgcaacttattactgtcaacagcttaatagttacccattcactttcggccctgggaccaaagtggatatcaaa

SEQ ID NO: 162 - protein, 107 aa, Artificial Sequence
  synthetic
DIQLTQSPSFLSASVGDRVTITCWASQGISSYLAWYQQKPGKAPKLLIHTASTLQSGVPSRFSGSGSGTEFTLTISNLQPEDFATYYCQQLNSYPFTFGPGTKVDIK

SEQ ID NO: 163 - DNA, 18 bp, Artificial Sequence
  synthetic
cagggcattagcagttat

SEQ ID NO: 164 - protein, 6 aa, Artificial Sequence
  synthetic
QGISSY

SEQ ID NO: 165 - DNA, 9 bp, Artificial Sequence
  synthetic
actgcatcc

SEQ ID NO: 166 - protein, 3 aa, Artificial Sequence
  synthetic
TAS

SEQ ID NO: 167 - DNA, 27 bp, Artificial Sequence
  synthetic
caacagcttaatagttacccattcact

SEQ ID NO: 168 - protein, 9 aa, Artificial Sequence
  synthetic
QQLNSYPFT

SEQ ID NO: 169 - DNA, 366 bp, Artificial Sequence
  synthetic
caggtgcagctggtggagtctgggggaggcgtggtccagcctggggggtccctgagactctcctgtgcagcctctggattcaccttcggtggccatgccatgcactgggtccgccaggctccaggcaaggggctggagtggctggcagttatatcatctgatggcagtaataaacagtatgcagattctgtgaagggccgattcaccatctccagggacaatcccaagaacacgctgtatctgcaaatgaacagtctgagagttggggacacggctatttattactgtgcgaaagaggtggcacctcgttattattattacggtctggacgtctggggccaagggaccacggtcaccgtctcctca

SEQ ID NO: 170 - protein, 122 aa, Artificial Sequence
  synthetic
QVQLVESGGGVVQPGGSLRLSCAASGFTFGGHAMHWVRQAPGKGLEWLAVISSDGSNKQYADSVKGRFTISRDNPKNTLYLQMNSLRVGDTAIYYCAKEVAPRYYYYGLDVWGQGTTVTVSS

SEQ ID NO: 171 - DNA, 24 bp, Artificial Sequence
  synthetic
ggattcaccttcggtggccatgcc

SEQ ID NO: 172 - protein, 8 aa, Artificial Sequence
  synthetic
GFTFGGHA

SEQ ID NO: 173 - DNA, 24 bp, Artificial Sequence
  synthetic
atatcatctgatggcagtaataaa

SEQ ID NO: 174 - protein, 8 aa, Artificial Sequence
  synthetic
ISSDGSNK

SEQ ID NO: 175 - DNA, 45 bp, Artificial Sequence
  synthetic
gcgaaagaggtggcacctcgttattattattacggtctggacgtc

SEQ ID NO: 176 - protein, 15 aa, Artificial Sequence
  synthetic
AKEVAPRYYYYGLDV

SEQ ID NO: 177 - DNA, 318 bp, Artificial Sequence
  synthetic
gacatccagatgacccagtctccatcctccctgtctgcatctgtgggagacagagtcaccatcacttgccgggcgagtcaggacattagcaattttttagcctggtatcagcagaaaccagggaaggttcctaaactcctgatctatactgcatccactttacaatcaggggtcccatctcggttcagtggcagtggatctgggacagatttcactctcaccgtcagcagcctacagcctgaagatgttgcaacttattactgtcaaaagtatgccggcgccctcactttcggccctgggaccaaagtggatatcaaa

SEQ ID NO: 178 - protein, 106 aa, Artificial Sequence
  synthetic
DIQMTQSPSSLSASVGDRVTITCRASQDISNFLAWYQQKPGKVPKLLIYTASTLQSGVPSRFSGSGSGTDFTLTVSSLQPEDVATYYCQKYAGALTFGPGTKVDIK

SEQ ID NO: 179 - DNA, 18 bp, Artificial Sequence
  synthetic
caggacattagcaatttt

SEQ ID NO: 180 - protein, 6 aa, Artificial Sequence
  synthetic
QDISNF

SEQ ID NO: 181 - DNA, 9 bp, Artificial Sequence
  synthetic
actgcatcc

SEQ ID NO: 182 - protein, 3 aa, Artificial Sequence
  synthetic
TAS

SEQ ID NO: 183 - DNA, 24 bp, Artificial Sequence
  synthetic
caaaagtatgccggcgccctcact

SEQ ID NO: 184 - protein, 8 aa, Artificial Sequence
  synthetic
QKYAGALT

SEQ ID NO: 185 - DNA, 357 bp, Artificial Sequence
  synthetic
gaggtgcagctggtggagtctgggggaggcttggcacagcctggggggtccctgagactctcctgtgcagcctctggattcacgtttagaagctatgccatgagctgggtccgccaggctccagggaaggggccggagtgggtctcaggtataggtggtaatggtgttaccacatactacgcagactccgtgaagggccggttcaccatctccagagacaattccaagaacacgctgtttctgcaaatgaatagcctgagagccgaggacacggccgtatattattgtgtgcaggggggtttaggtggttattttacaggctactggggccagggaaccctggtcaccgtctcctca

SEQ ID NO: 186 - protein, 119 aa, Artificial Sequence
  synthetic
EVQLVESGGGLAQPGGSLRLSCAASGFTFRSYAMSWVRQAPGKGPEWVSGIGGNGVTTYYADSVKGRFTISRDNSKNTLFLQMNSLRAEDTAVYYCVQGGLGGYFTGYWGQGTLVTVSS

SEQ ID NO: 187 - DNA, 24 bp, Artificial Sequence
  synthetic
ggattcacgtttagaagctatgcc

SEQ ID NO: 188 - protein, 8 aa, Artificial Sequence
  synthetic
GFTFRSYA

SEQ ID NO: 189 - DNA, 24 bp, Artificial Sequence
  synthetic
ataggtggtaatggtgttaccaca

SEQ ID NO: 190 - protein, 8 aa, Artificial Sequence
  synthetic
IGGNGVTT

SEQ ID NO: 191 - DNA, 36 bp, Artificial Sequence
  synthetic
gtgcaggggggtttaggtggttattttacaggctac

SEQ ID NO: 192 - protein, 12 aa, Artificial Sequence
  synthetic
VQGGLGGYFTGY

SEQ ID NO: 193 - DNA, 321 bp, Artificial Sequence
  synthetic
gacatccagatgacccagtctccatcctccctgtctgcatctgtaggagacagagtcaccatcacttgccgggcaagtcagagtattagtacctatttaaattggtatcagcagaatccagggaaagcccctaaactcctgatctttgatgcatccagtttgcaaagtggggtcccatcaaggttcagtggcagtggatctgggacagatttcactctcaccatcagaggtctgcaacctgaagattttgcaacttactactgtcaacagagttacagtgccccgctcactttcggcggagggaccaaggtggagatcaaa

SEQ ID NO: 194 - protein, 107 aa, Artificial Sequence
  synthetic
DIQMTQSPSSLSASVGDRVTITCRASQSISTYLNWYQQNPGKAPKLLIFDASSLQSGVPSRFSGSGSGTDFTLTIRGLQPEDFATYYCQQSYSAPLTFGGGTKVEIK

SEQ ID NO: 195 - DNA, 18 bp, Artificial Sequence
  synthetic
cagagtattagtacctat

SEQ ID NO: 196 - protein, 6 aa, Artificial Sequence
  synthetic
QSISTY

SEQ ID NO: 197 - DNA, 9 bp, Artificial Sequence
  synthetic
gatgcatcc

SEQ ID NO: 198 - protein, 3 aa, Artificial Sequence
  synthetic
DAS

SEQ ID NO: 199 - DNA, 27 bp, Artificial Sequence
  synthetic
caacagagttacagtgccccgctcact

SEQ ID NO: 200 - protein, 9 aa, Artificial Sequence
  synthetic
QQSYSAPLT

SEQ ID NO: 201 - DNA, 357 bp, Artificial Sequence
  synthetic
caggtgcagctggtggagtctgggggaggcgtggtccagcctgggaggtccctgagactctcctgtgcagcgtctggattcaccttcagtggttatggcatgcactgggtccgccaggctccaggcaaggggctggagtgggtggcacttatatggcttgatggaagtaatgactactatgcagactccgtgaagggccgattcaccatctccagagacaattccaagaacacgttatatctgcaaatgaacagactgagagccgaggacacggctgtgtattactgtgcgagagatggcccggttgctgctatacccgactactggggccagggaaccctggtcaccgtctcctca

SEQ ID NO: 202 - protein, 119 aa, Artificial Sequence
  synthetic
QVQLVESGGGVVQPGRSLRLSCAASGFTFSGYGMHWVRQAPGKGLEWVALIWLDGSNDYYADSVKGRFTISRDNSKNTLYLQMNRLRAEDTAVYYCARDGPVAAIPDYWGQGTLVTVSS

SEQ ID NO: 203 - DNA, 24 bp, Artificial Sequence
  synthetic
ggattcaccttcagtggttatggc

SEQ ID NO: 204 - protein, 8 aa, Artificial Sequence
  synthetic
GFTFSGYG

SEQ ID NO: 205 - DNA, 24 bp, Artificial Sequence
  synthetic
atatggcttgatggaagtaatgac

SEQ ID NO: 206 - protein, 8 aa, Artificial Sequence
  synthetic
IWLDGSND

SEQ ID NO: 207 - DNA, 36 bp, Artificial Sequence
  synthetic
gcgagagatggcccggttgctgctatacccgactac

SEQ ID NO: 208 - protein, 12 aa, Artificial Sequence
  synthetic
ARDGPVAAIPDY

SEQ ID NO: 209 - DNA, 321 bp, Artificial Sequence
  synthetic
gacatccagatgacccagtctccttccaccctgtctgcatctgtaggagacagagtcaccatcacttgccgggccagtcagagtattagtaggtggttggcctggtatcagctgaaaccagggaaagcccctaagctcctgatctataaggcgtctagtttagaaagtggggtcccatcaaggttcagcggcagtggatctgggacagacttcactctcaccatcagcagcctgcaacctgatgattttgcaacttattactgccaacagtataatacttattcgtacacttttggccaggggaccaagctggagatcaaa

SEQ ID NO: 210 - protein, 107 aa, Artificial Sequence
  synthetic
DIQMTQSPSTLSASVGDRVTITCRASQSISRWLAWYQLKPGKAPKLLIYKASSLESGVPSRFSGSGSGTDFTLTISSLQPDDFATYYCQQYNTYSYTFGQGTKLEIK

SEQ ID NO: 211 - DNA, 18 bp, Artificial Sequence
  synthetic
cagagtattagtaggtgg

SEQ ID NO: 212 - protein, 6 aa, Artificial Sequence
  synthetic
QSISRW

SEQ ID NO: 213 - DNA, 9 bp, Artificial Sequence
  synthetic
aaggcgtct

SEQ ID NO: 214 - protein, 3 aa, Artificial Sequence
  synthetic
KAS

SEQ ID NO: 215 - DNA, 27 bp, Artificial Sequence
  synthetic
caacagtataatacttattcgtacact

SEQ ID NO: 216 - protein, 9 aa, Artificial Sequence
  synthetic
QQYNTYSYT

SEQ ID NO: 217 - DNA, 363 bp, Artificial Sequence
  synthetic
gaggtgcagctggtggagtctgggggaggtgtggtacggcctggggggtccctgagactctcctgtgcagcctctggattcacctttgatgaatatggcatgacttgggtccgccaagttccagggaaggggctggagtgggtctctggtattacttggaatggtggtttcacagattatacagactctgtgaagggccgattcaccagctccagagacaacgccaagaactccctgtatctgcaaatgaacagtctgagagccgaggacacggccttgtattactgtgcgagagatggatatagcagctcgtggggggcttatgatatatggggccaagggacaatggtcaccgtctcttca

SEQ ID NO: 218 - protein, 121 aa, Artificial Sequence
  synthetic
EVQLVESGGGVVRPGGSLRLSCAASGFTFDEYGMTWVRQVPGKGLEWVSGITWNGGFTDYTDSVKGRFTSSRDNAKNSLYLQMNSLRAEDTALYYCARDGYSSSWGAYDIWGQGTMVTVSS

SEQ ID NO: 219 - DNA, 24 bp, Artificial Sequence
  synthetic
ggattcacctttgatgaatatggc

SEQ ID NO: 220 - protein, 8 aa, Artificial Sequence
  synthetic
GFTFDEYG

SEQ ID NO: 221 - DNA, 24 bp, Artificial Sequence
  synthetic
attacttggaatggtggtttcaca

SEQ ID NO: 222 - protein, 8 aa, Artificial Sequence
  synthetic
ITWNGGFT

SEQ ID NO: 223 - DNA, 42 bp, Artificial Sequence
  synthetic
gcgagagatggatatagcagctcgtggggggcttatgatata

SEQ ID NO: 224 - protein, 14 aa, Artificial Sequence
  synthetic
ARDGYSSSWGAYDI

SEQ ID NO: 225 - DNA, 321 bp, Artificial Sequence
  synthetic
gacatccagatgacccagtctccatcatccctgtctgcatctgtgggagacagagtcaccatcacttgccgggcaagtcagagcattagcacctatttaaattggtatcagcagaaaccagggaaagcccctaagctcctgatctatgctgcatccagtttgcaaagtggggtcccattaaggttcagtggcagtggatctgggactgatttcactctcaccatcagcagtctgcaacctgaagattttgcaagttatttctgtcaacagagttacagtaccccgtacacttttggccaggggaccaagctggagatcaaa

SEQ ID NO: 226 - protein, 107 aa, Artificial Sequence
  synthetic
DIQMTQSPSSLSASVGDRVTITCRASQSISTYLNWYQQKPGKAPKLLIYAASSLQSGVPLRFSGSGSGTDFTLTISSLQPEDFASYFCQQSYSTPYTFGQGTKLEIK

SEQ ID NO: 227 - DNA, 18 bp, Artificial Sequence
  synthetic
cagagcattagcacctat

SEQ ID NO: 228 - protein, 6 aa, Artificial Sequence
  synthetic
QSISTY

SEQ ID NO: 229 - DNA, 9 bp, Artificial Sequence
  synthetic
gctgcatcc

SEQ ID NO: 230 - protein, 3 aa, Artificial Sequence
  synthetic
AAS

SEQ ID NO: 231 - DNA, 27 bp, Artificial Sequence
  synthetic
caacagagttacagtaccccgtacact

SEQ ID NO: 232 - protein, 9 aa, Artificial Sequence
  synthetic
QQSYSTPYT

SEQ ID NO: 233 - DNA, 360 bp, Artificial Sequence
  synthetic
gaagtgcagctggtggagtctgggggaggcgtggtacagcctggggggtccctgagactctcctgtgcagcctctggattcacctttaatgattatgccatgcactgggtccgtcaagctccagggaagggtctggagtgggtctctcttattagtggagatggtggtaacacatactatgcagactctgtgaagggccgactcaccatctccagagacaacagcaaaaactccctgtatctgcaaatgaacagtctgagaacagaggacaccgccttatattactgtgcaaaagataagggctggaacttcggttacttcgatctctggggccgtggcaccctggtcactgtctcctca

SEQ ID NO: 234 - protein, 120 aa, Artificial Sequence
  synthetic
EVQLVESGGGVVQPGGSLRLSCAASGFTFNDYAMHWVRQAPGKGLEWVSLISGDGGNTYYADSVKGRLTISRDNSKNSLYLQMNSLRTEDTALYYCAKDKGWNFGYFDLWGRGTLVTVSS

SEQ ID NO: 235 - DNA, 24 bp, Artificial Sequence
  synthetic
ggattcacctttaatgattatgcc

SEQ ID NO: 236 - protein, 8 aa, Artificial Sequence
  synthetic
GFTFNDYA

SEQ ID NO: 237 - DNA, 24 bp, Artificial Sequence
  synthetic
attagtggagatggtggtaacaca

SEQ ID NO: 238 - protein, 8 aa, Artificial Sequence
  synthetic
ISGDGGNT

SEQ ID NO: 239 - DNA, 39 bp, Artificial Sequence
  synthetic
gcaaaagataagggctggaacttcggttacttcgatctc

SEQ ID NO: 240 - protein, 13 aa, Artificial Sequence
  synthetic
AKDKGWNFGYFDL

SEQ ID NO: 241 - DNA, 327 bp, Artificial Sequence
  synthetic
gacatccagatgacccagtctccatcctccctgtctacatctgtgggagacagagtcaccatcacttgccgggcaagtcagaacattgacacctatttaaattggtatcagcagaaaccagggaaagcccctaaactcctgatctatgatgcatccagtttacaaagtggggtcccatcacggttcagtggcagcggatctgggacagatttcactctcaccatcaccagtctgcaacctgaagattttgccacttactactgtcaacagaatgacaatattcttcaccctctcactttcggcggagggaccaaggtggagatcaaa

SEQ ID NO: 242 - protein, 109 aa, Artificial Sequence
  synthetic
DIQMTQSPSSLSTSVGDRVTITCRASQNIDTYLNWYQQKPGKAPKLLIYDASSLQSGVPSRFSGSGSGTDFTLTITSLQPEDFATYYCQQNDNILHPLTFGGGTKVEIK

SEQ ID NO: 243 - DNA, 18 bp, Artificial Sequence
  synthetic
cagaacattgacacctat

SEQ ID NO: 244 - protein, 6 aa, Artificial Sequence
  synthetic
QNIDTY

SEQ ID NO: 245 - DNA, 9 bp, Artificial Sequence
  synthetic
gatgcatcc

SEQ ID NO: 246 - protein, 3 aa, Artificial Sequence
  synthetic
DAS

SEQ ID NO: 247 - DNA, 33 bp, Artificial Sequence
  synthetic
caacagaatgacaatattcttcaccctctcact

SEQ ID NO: 248 - protein, 11 aa, Artificial Sequence
  synthetic
QQNDNILHPLT

SEQ ID NO: 249 - DNA, 369 bp, Artificial Sequence
  synthetic
gaggtgcagctggtggagtctgggggaggcttggtccaaccgggggggtccctgagactctcctgtgcagcctctggattccactctaatagatattggatggactgggtccgccaggctccagggaaggggctggagtgggtggccaacataaagcaagatggaagtgaggaaaactatgtggactctgtgaagggccgattcaccatctccagagacaacgccaagaactcactttatctgcaaatgaacagcctgagagccgaggacacggctgtgtattactgtgcgagagatcgaagcacctcgtgggtcccttactggttcttcgatctctggggccgtggcaccctggtcactgtctcctca

SEQ ID NO: 250 - protein, 123 aa, Artificial Sequence
  synthetic
EVQLVESGGGLVQPGGSLRLSCAASGFHSNRYWMDWVRQAPGKGLEWVANIKQDGSEENYVDSVKGRFTISRDNAKNSLYLQMNSLRAEDTAVYYCARDRSTSWVPYWFFDLWGRGTLVTVSS

SEQ ID NO: 251 - DNA, 24 bp, Artificial Sequence
  synthetic
ggattccactctaatagatattgg

SEQ ID NO: 252 - protein, 8 aa, Artificial Sequence
  synthetic
GFHSNRYW

SEQ ID NO: 253 - DNA, 24 bp, Artificial Sequence
  synthetic
ataaagcaagatggaagtgaggaa

SEQ ID NO: 254 - protein, 8 aa, Artificial Sequence
  synthetic
IKQDGSEE

SEQ ID NO: 255 - DNA, 48 bp, Artificial Sequence
  synthetic
gcgagagatcgaagcacctcgtgggtcccttactggttcttcgatctc

SEQ ID NO: 256 - protein, 16 aa, Artificial Sequence
  synthetic
ARDRSTSWVPYWFFDL

SEQ ID NO: 257 - DNA, 324 bp, Artificial Sequence
  synthetic
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 258 - protein, 108 aa, Artificial Sequence
  synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 259 - DNA, 18 bp, Artificial Sequence
  synthetic
cagagcattagcagctat

SEQ ID NO: 260 - protein, 6 aa, Artificial Sequence
  synthetic
QSISSY

SEQ ID NO: 261 - DNA, 9 bp, Artificial Sequence
  synthetic
gctgcatcc

SEQ ID NO: 262 - protein, 3 aa, Artificial Sequence
  synthetic
AAS

SEQ ID NO: 263 - DNA, 30 bp, Artificial Sequence
  synthetic
caacagagttacagtacccctccgatcacc

SEQ ID NO: 264 - protein, 10 aa, Artificial Sequence
  synthetic
QQSYSTPPIT

SEQ ID NO: 265 - DNA, 360 bp, Artificial Sequence
  synthetic
gaagtgcagctggtggagtctgggggaggcgtggtacagcggggggagtccctgagactctcctgttcagcctctgacttcatctttaaagattatgccatgtactgggtccgtcaaattccagggaagggtctagagtggatctctcttattagtggtgatggtgacactacatggtatggagactctgtgaagggccgattcaccatctccagagacaacaacgaaaactccctctttctgcaaatgaacgatctgagaactgaggacaccgccatgtactactgtgcaagagatatggggtggaacttctttcagttgcaatactggggccagggaaccctggtcaccgtctcctca

SEQ ID NO: 266 - protein, 120 aa, Artificial Sequence
  synthetic
EVQLVESGGGVVQRGESLRLSCSASDFIFKDYAMYWVRQIPGKGLEWISLISGDGDTTWYGDSVKGRFTISRDNNENSLFLQMNDLRTEDTAMYYCARDMGWNFFQLQYWGQGTLVTVSS

SEQ ID NO: 267 - DNA, 24 bp, Artificial Sequence
  synthetic
gacttcatctttaaagattatgcc

SEQ ID NO: 268 - protein, 8 aa, Artificial Sequence
  synthetic
DFIFKDYA

SEQ ID NO: 269 - DNA, 24 bp, Artificial Sequence
  synthetic
attagtggtgatggtgacactaca

SEQ ID NO: 270 - protein, 8 aa, Artificial Sequence
  synthetic
ISGDGDTT

SEQ ID NO: 271 - DNA, 39 bp, Artificial Sequence
  synthetic
gcaagagatatggggtggaacttctttcagttgcaatac

SEQ ID NO: 272 - protein, 13 aa, Artificial Sequence
  synthetic
ARDMGWNFFQLQY

SEQ ID NO: 273 - DNA, 361 bp, Artificial Sequence
  synthetic
dcaggtgcagctgcaggagtcgggccccgcactggtgaagccttcacagaccctgtccctcacctgcactgtctctggtggctccatcatcagaggtagtacctactggagttgggtccgccaattcccagggaagggcctggagtggattggatacagttattacagtgggaccgcctactataatccgtccctcgagagtcgagctaccatttctgtagacacgtctaagaaccagttctccctgaacctgaagtctgtgacggccgcggacacggccgtgtattattgtacaagagaaataggagtggctggtctctttgacatctggggccagggaaccctggtcaccgtctcctca

SEQ ID NO: 274 - protein, 120 aa, Artificial Sequence
  synthetic
QVQLQESGPALVKPSQTLSLTCTVSGGSIIRGSTYWSWVRQFPGKGLEWIGYSYYSGTAYYNPSLESRATISVDTSKNQFSLNLKSVTAADTAVYYCTREIGVAGLFDIWGQGTLVTVSS

SEQ ID NO: 275 - DNA, 30 bp, Artificial Sequence
  synthetic
ggtggctccatcatcagaggtagtacctac

SEQ ID NO: 276 - protein, 10 aa, Artificial Sequence
  synthetic
GGSIIRGSTY

SEQ ID NO: 277 - DNA, 21 bp, Artificial Sequence
  synthetic
agttattacagtgggaccgcc

SEQ ID NO: 278 - protein, 7 aa, Artificial Sequence
  synthetic
SYYSGTA

SEQ ID NO: 279 - DNA, 36 bp, Artificial Sequence
  synthetic
acaagagaaataggagtggctggtctctttgacatc

SEQ ID NO: 280 - protein, 12 aa, Artificial Sequence
  synthetic
TREIGVAGLFDI

SEQ ID NO: 281 - DNA, 324 bp, Artificial Sequence
  synthetic
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaag

SEQ ID NO: 282 - protein, 108 aa, Artificial Sequence
  synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 283 - DNA, 36 bp, Artificial Sequence
  synthetic
agagcaagtcagtcagtctctagctcttatctcgcc

SEQ ID NO: 284 - protein, 12 aa, Artificial Sequence
  synthetic
RASQSVSSSYLA

SEQ ID NO: 285 - DNA, 21 bp, Artificial Sequence
  synthetic
ggggcaagttccagggccacc

SEQ ID NO: 286 - protein, 7 aa, Artificial Sequence
  synthetic
GASSRAT

SEQ ID NO: 287 - DNA, 27 bp, Artificial Sequence
  synthetic
caacagtacggaagcagcccgtggacg

SEQ ID NO: 288 - protein, 9 aa, Artificial Sequence
  synthetic
QQYGSSPWT

SEQ ID NO: 289 - DNA, 357 bp, Artificial Sequence
  synthetic
caggagcagttggtgcagtctggggctgaggtgaagaagcctggggcctcagtgaaggtctcctgtaaggcttctggatacaccttcaccggctactatatacattgggtgcgacaggcccctggactagggcttgaatggatgggatggatcaaccctaacagtggtggcacaaaatatgcacagaagtttcagggcagggtcaccatgaccagggacacgtccatcaatacagcctacatggagctgaaaagactgaaatctgacgactcggccgtatattactgtgcgagagacgcccctccccatgatgtttttgatatctggggccaagggacattggtcaccgtctcttca

SEQ ID NO: 290 - protein, 119 aa, Artificial Sequence
  synthetic
QEQLVQSGAEVKKPGASVKVSCKASGYTFTGYYIHWVRQAPGLGLEWMGWINPNSGGTKYAQKFQGRVTMTRDTSINTAYMELKRLKSDDSAVYYCARDAPPHDVFDIWGQGTLVTVSS

SEQ ID NO: 291 - DNA, 24 bp, Artificial Sequence
  synthetic
ggatacaccttcaccggctactat

SEQ ID NO: 292 - protein, 8 aa, Artificial Sequence
  synthetic
GYTFTGYY

SEQ ID NO: 293 - DNA, 24 bp, Artificial Sequence
  synthetic
atcaaccctaacagtggtggcaca

SEQ ID NO: 294 - protein, 8 aa, Artificial Sequence
  synthetic
INPNSGGT

SEQ ID NO: 295 - DNA, 36 bp, Artificial Sequence
  synthetic
gcgagagacgcccctccccatgatgtttttgatatc

SEQ ID NO: 296 - protein, 12 aa, Artificial Sequence
  synthetic
ARDAPPHDVFDI

SEQ ID NO: 297 - DNA, 321 bp, Artificial Sequence
  synthetic
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaattggggtcccatcaaggttcagcggcagtggatctgggacagaattcactctcacaatcagcagcctgcagcctgaagattttgcaacttattactgtctacagcataatagttacccgctcactttcggcggagggaccaaggtggagatcaaa

SEQ ID NO: 298 - protein, 107 aa, Artificial Sequence
  synthetic
DIQMTQSPSSLSASVGDRVTITCRASQGIRNDLGWYQQKPGKAPKRLIYAASSLQIGVPSRFSGSGSGTEFTLTISSLQPEDFATYYCLQHNSYPLTFGGGTKVEIK

SEQ ID NO: 299 - DNA, 18 bp, Artificial Sequence
  synthetic
cagggcattagaaatgat

SEQ ID NO: 300 - protein, 6 aa, Artificial Sequence
  synthetic
QGIRND

SEQ ID NO: 301 - DNA, 9 bp, Artificial Sequence
  synthetic
gctgcatcc

SEQ ID NO: 302 - protein, 3 aa, Artificial Sequence
  synthetic
AAS

SEQ ID NO: 303 - DNA, 27 bp, Artificial Sequence
  synthetic
ctacagcataatagttacccgctcact

SEQ ID NO: 304 - protein, 9 aa, Artificial Sequence
  synthetic
LQHNSYPLT

SEQ ID NO: 305 - DNA, 357 bp, Artificial Sequence
  synthetic
caggtgcagctgcaagagtcgggcccaggactggtgaagccttcacagaccctgtccctcacctgcactgtctctggtggctccatcagtagtggtgcttaccactggagctggatccgccagcacccagggaagggcctagagtggattggatacatctattacaatggggacacctactataatccgtccctcaagagtcgcgttaccatttcagtggacacgtctaagaaccaattcttcctgaaggtgacctctgtgactgccgcggacacggccatgtattactgtgcgggagaaaagcagctgactgcttttgatatctggggccaagggacattggtcaccgtctcttca

SEQ ID NO: 306 - protein, 119 aa, Artificial Sequence
  synthetic
QVQLQESGPGLVKPSQTLSLTCTVSGGSISSGAYHWSWIRQHPGKGLEWIGYIYYNGDTYYNPSLKSRVTISVDTSKNQFFLKVTSVTAADTAMYYCAGEKQLTAFDIWGQGTLVTVSS

SEQ ID NO: 307 - DNA, 30 bp, Artificial Sequence
  synthetic
ggtggctccatcagtagtggtgcttaccac

SEQ ID NO: 308 - protein, 10 aa, Artificial Sequence
  synthetic
GGSISSGAYH

SEQ ID NO: 309 - DNA, 21 bp, Artificial Sequence
  synthetic
atctattacaatggggacacc

SEQ ID NO: 310 - protein, 7 aa, Artificial Sequence
  synthetic
IYYNGDT

SEQ ID NO: 311 - DNA, 33 bp, Artificial Sequence
  synthetic
gcgggagaaaagcagctgactgcttttgatatc

SEQ ID NO: 312 - protein, 11 aa, Artificial Sequence
  synthetic
AGEKQLTAFDI

SEQ ID NO: 313 - DNA, 321 bp, Artificial Sequence
  synthetic
gtcatccagatgacccagtctccatcctccctgtctgcatctgttggagacagagtcaccataacttgccgggcgagtcaggacattaataattttttaaattggtatcaacagaaattagggaaagcccctaaactcctgatctccgatgcatccaatttgcagacaggagtcccgtcaaggttcagtggaagtggatctgggacagattttactttcaccatcagcagcctgcagcctgaagatattgctgcatattactgtcaacaatatgatcatttcccgtatacttttggccaggggaccagactggagaacaat

SEQ ID NO: 314 - protein, 107 aa, Artificial Sequence
  synthetic
VIQMTQSPSSLSASVGDRVTITCRASQDINNFLNWYQQKLGKAPKLLISDASNLQTGVPSRFSGSGSGTDFTFTISSLQPEDIAAYYCQQYDHFPYTFGQGTRLENN

SEQ ID NO: 315 - DNA, 18 bp, Artificial Sequence
  synthetic
caggacattaataatttt

SEQ ID NO: 316 - protein, 6 aa, Artificial Sequence
  synthetic
QDINNF

SEQ ID NO: 317 - DNA, 9 bp, Artificial Sequence
  synthetic
gatgcatcc

SEQ ID NO: 318 - protein, 3 aa, Artificial Sequence
  synthetic
DAS

SEQ ID NO: 319 - DNA, 27 bp, Artificial Sequence
  synthetic
caacaatatgatcatttcccgtatact

SEQ ID NO: 320 - protein, 9 aa, Artificial Sequence
  synthetic
QQYDHFPYT

SEQ ID NO: 321 - DNA, 357 bp, Artificial Sequence
  synthetic
gaggtgcagttggtggagtctgggggaggtgtggttcggcctggggggtccctgagactctcctgtgcagcctctggattcacctttgatgattatggcatgacctgggtccgccaagctccagggaaggggctggagtgggtctctggtattaattggaatggcgatagcacagagtattcagactctgtgaagggccgattcaccatctccagagacaacgccaagaactccctgtatctgcaaatgaacagtctgagagccgaggacacggccttctatcactgtgcgagagagaataactggaacttctactttgactactggggccagggaaccctggtcaccgtctcctca

SEQ ID NO: 322 - protein, 119 aa, Artificial Sequence
  synthetic
EVQLVESGGGVVRPGGSLRLSCAASGFTFDDYGMTWVRQAPGKGLEWVSGINWNGDSTEYSDSVKGRFTISRDNAKNSLYLQMNSLRAEDTAFYHCARENNWNFYFDYWGQGTLVTVSS

SEQ ID NO: 323 - DNA, 24 bp, Artificial Sequence
  synthetic
ggattcacctttgatgattatggc

SEQ ID NO: 324 - protein, 8 aa, Artificial Sequence
  synthetic
GFTFDDYG

SEQ ID NO: 325 - DNA, 24 bp, Artificial Sequence
  synthetic
attaattggaatggcgatagcaca

SEQ ID NO: 326 - protein, 8 aa, Artificial Sequence
  synthetic
INWNGDST

SEQ ID NO: 327 - DNA, 36 bp, Artificial Sequence
  synthetic
gcgagagagaataactggaacttctactttgactac

SEQ ID NO: 328 - protein, 12 aa, Artificial Sequence
  synthetic
ARENNWNFYFDY

SEQ ID NO: 329 - DNA, 321 bp, Artificial Sequence
  synthetic
gaaatagtgatgacgcagtctccagccaccctgtctgtgtctcgaggggaaagagccaccctctcctgtagggccagtcagagtgttagcagcaacttagcctggtaccagcagaaacttggccaggctcccaggctcctcatctatggtgcatccaccagggccactggtatcccagccaggttcagtggcagtgggtctgggacagagttcactctcaccatcagcagcctgcagtctgaagattttgcagtttattattgtcagcagtataataactggccgtggacgttcggccaagggaccaaggtggaaatcaaa

SEQ ID NO: 330 - protein, 107 aa, Artificial Sequence
  synthetic
EIVMTQSPATLSVSRGERATLSCRASQSVSSNLAWYQQKLGQAPRLLIYGASTRATGIPARFSGSGSGTEFTLTISSLQSEDFAVYYCQQYNNWPWTFGQGTKVEIK

SEQ ID NO: 331 - DNA, 18 bp, Artificial Sequence
  synthetic
cagagtgttagcagcaac

SEQ ID NO: 332 - protein, 6 aa, Artificial Sequence
  synthetic
QSVSSN

SEQ ID NO: 333 - DNA, 9 bp, Artificial Sequence
  synthetic
ggtgcatcc

SEQ ID NO: 334 - protein, 3 aa, Artificial Sequence
  synthetic
GAS

SEQ ID NO: 335 - DNA, 27 bp, Artificial Sequence
  synthetic
cagcagtataataactggccgtggacg

SEQ ID NO: 336 - protein, 9 aa, Artificial Sequence
  synthetic
QQYNNWPWT

SEQ ID NO: 337 - DNA, 351 bp, Artificial Sequence
  synthetic
caggtccacctggtacagtctggggctgaggtgaagaagcctggggcctcagtgaaggtctcctgcaaggtttccggaaacaccctcactgaattatccatgcactgggtgcgacaggctcctggaaaagggcttgagtggatgggaggttttgatcctgaagatggtgacacaatctactcacagaagttccagggcagagtcaccttgaccgaggacacatctacagacacagcctacatggagctgagcagcctgagatctgaggacacggccgtgtattactgttcaacagtggggggacctacctctgactgctggggccagggaaccctggtcaccgtctcctca

SEQ ID NO: 338 - protein, 117 aa, Artificial Sequence
  synthetic
QVHLVQSGAEVKKPGASVKVSCKVSGNTLTELSMHWVRQAPGKGLEWMGGFDPEDGDTIYSQKFQGRVTLTEDTSTDTAYMELSSLRSEDTAVYYCSTVGGPTSDCWGQGTLVTVSS

SEQ ID NO: 339 - DNA, 24 bp, Artificial Sequence
  synthetic
ggaaacaccctcactgaattatcc

SEQ ID NO: 340 - protein, 8 aa, Artificial Sequence
  synthetic
GNTLTELS

SEQ ID NO: 341 - DNA, 24 bp, Artificial Sequence
  synthetic
tttgatcctgaagatggtgacaca

SEQ ID NO: 342 - protein, 8 aa, Artificial Sequence
  synthetic
FDPEDGDT

SEQ ID NO: 343 - DNA, 30 bp, Artificial Sequence
  synthetic
tcaacagtggggggacctacctctgactgc

SEQ ID NO: 344 - protein, 10 aa, Artificial Sequence
  synthetic
STVGGPTSDC

SEQ ID NO: 345 - DNA, 321 bp, Artificial Sequence
  synthetic
gacatccagatgacccagtctccatcctccctgtctgcatctgtaggagacagagtcaccatcacttgccaggcgagtcaggacattagcaactatttaaattggtatcagcagaaaccagggaaagcccctaaggtcctgatcttcgatgcatccaatttagaaccaggggtcccatcaaggttcagtggaagtggatctgggacagattttactttcaccatcatcagcctgcagcctgaagatattgcaacatattactgtcaacaatatgataatctcccgatcaccttcggccaggggacacgactggacattaaa

SEQ ID NO: 346 - protein, 107 aa, Artificial Sequence
  synthetic
DIQMTQSPSSLSASVGDRVTITCQASQDISNYLNWYQQKPGKAPKVLIFDASNLEPGVPSRFSGSGSGTDFTFTIISLQPEDIATYYCQQYDNLPITFGQGTRLDIK

SEQ ID NO: 347 - DNA, 18 bp, Artificial Sequence
  synthetic
caggacattagcaactat

SEQ ID NO: 348 - protein, 6 aa, Artificial Sequence
  synthetic
QDISNY

SEQ ID NO: 349 - DNA, 9 bp, Artificial Sequence
  synthetic
gatgcatcc

SEQ ID NO: 350 - protein, 3 aa, Artificial Sequence
  synthetic
DAS

SEQ ID NO: 351 - DNA, 27 bp, Artificial Sequence
  synthetic
caacaatatgataatctcccgatcacc

SEQ ID NO: 352 - protein, 9 aa, Artificial Sequence
  synthetic
QQYDNLPIT

SEQ ID NO: 353 - protein, 9 aa, Homo sapiens
NMATGMDSW

SEQ ID NO: 354 - protein, 20 aa, Homo sapiens
WEVHLVPRRKQLQFALPDSL

SEQ ID NO: 355 - protein, 18 aa, Homo sapiens
KDMQLGRLHMKTLLPVSK

SEQ ID NO: 356 - protein, 9 aa, Homo sapiens
NDETCEQRA

SEQ ID NO: 357 - protein, 7 aa, Homo sapiens
SHKDMQL

SEQ ID NO: 358 - RNA, 23 nt, Artificial Sequence
  siRNA oligonucleotide
uauuauaaaaauaucuugcuuuu

SEQ ID NO: 359 - RNA, 21 nt, Artificial Sequence
  siRNA oligonucleotide
aagcaagauauuuuuauaaua

SEQ ID NO: 360 - RNA, 21 nt, Artificial Sequence
  siRNA oligonucleotide
aagcaagauauuuuuauaaua

SEQ ID NO: 361 - DNA, 25 bp, Artificial Sequence
  siRNA oligonucleotide
uauuauaaaaauaucuugcuuuutt

SEQ ID NO: 362 - protein, 446 aa, Homo sapiens
EVQLVQSGAEVKKPGSSVKVSCKASGGTFSSYAISWVRQAPGQGLEWMGGIGPFFGTANYAQKFQGRVTITADESTSTAYMELSSLRSEDTAVYYCARDTPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 363 - protein, 214 aa, Homo sapiens
SYELTQPLSVSVALGQTARITCSGDSIPNYYVYWYQQKPGQAPVLVIYDDSNRPSGIPERFSGSNSGNTATLTISRAQAGDEADYYCQSFDSSLNAEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 364 - protein, 448 aa, Artificial Sequence
  Humanized mouse antibody Ig
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 365 - protein, 214 aa, Artificial Sequence [sequence omitted]

SEQ ID NO: 366 - protein, 448 aa, Artificial Sequence
  Humanized mouse antibody Ig
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 367 - protein, 214 aa, Artificial Sequence [sequence omitted]

SEQ ID NO: 368 - protein, 447 aa, Homo sapiens
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 369 - protein, 214 aa, Homo sapiens
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC